(19) United States
(12) Patent Application Publication
(10) Pub. No.: US 2024/0294985 A1
(43) Pub. Date: Sep. 5, 2024

(54) PREECLAMPSIA DIAGNOSIS

(71) Applicant: Katholieke Universiteit Leuven, Leuven (BE)

(72) Inventors: Bernard Thienpont, Kessel-Lo (BE); Marie DE Borre, Mechelen (BE); Qian Yu, Heverlee (BE); Huiwen CHE, Leuven (BE); Kristel Van Calsteren, Wezemaal (BE)

(21) Appl. No.: 18/282,138

(22) PCT Filed: Mar. 15, 2022
(86) PCT No.: PCT/EP2022/056679

§ 371 Date: Sep. 14, 2023

(30) Foreign Application Priority Data
Mar. 15, 2021 (EP) 21162527.2

(51) Int. Cl. C12Q 1/6883

(52) U.S. Cl. CPC C12Q 1/6883; C12Q 2600/154

(57) ABSTRACT

The present invention relates to a method for the prediction of the risk of developing preeclampsia in a pregnant human subject, wherein said subject's gestational age is under 140 days, comprising the steps of: (a) measuring in a sample from said subject a plurality of loci-specific DNA methylation levels; (b) deriving from said measurement a DNA methylation profile for said subject (c) comparing said DNA methylation profile to one or more reference DNA methylation profile(s) corresponding to preeclampsia risk group; and (d) assigning on the basis of said comparison said subject to a preeclampsia risk group, thereby predicting the risk of developing preeclampsia in said subject.

### FIELD OF THE INVENTION

The present invention relates to methods for the prediction of the risk of developing preeclampsia. In particular the present invention relates to use of the measure of DNA methylation level for the presymptomatic prediction of the risk of developing preeclampsia.

### BACKGROUND OF THE INVENTION

Preeclampsia is a prevalent source of intra-uterine growth retardation (IUGR), premature delivery and low birth weight. It is the third leading cause for peripartal mortality and morbidity in expectant mothers worldwide, and it is associated with a significant societal burden because of the extensive perinatal care it requires and the potential complications in the offspring later in life. Although the pathology is thought to be initiated already in the early weeks of pregnancy, symptoms of new onset hypertension and proteinuria are only evident from the second half of the pregnancy. In order to prevent the acute and chronic consequences of preeclampsia, early identification of pregnant women at risk is essential. For example, administration of low-dose aspirin to high-risk women before 16 weeks of gestation significantly reduces the incidence of preterm preeclampsia and IUGR. In this context, a prognostic test would have a strong clinical impact by both enabling closer monitoring and early pharmacological intervention when necessary.

The pathogenesis of preeclampsia is incompletely understood, but likely multifactorial, with preeclampsia risk being influenced by BMI, parity and other factors. A number of maternal characteristics and risk factors associated with the development of preeclampsia have been used to develop several first trimester screening models to identify women at risk for developing preeclampsia. Examples of risk factors include a previous pregnancy complicated by preeclampsia, or twin pregnancies. However, screening models that are solely based on maternal demographic characteristics and medical history typically have a low detection rate and are therefore inadequate for effective prediction. The placenta appears critical in the pathophysiology of preeclampsia, as (i) presence of placental tissue is sufficient for development of the disease and (ii) preeclampsia is cured within days or weeks after delivery of the placenta. It is likely that the pathogenic process is initiated during the first trimester, long before clinical manifestations. Indeed, at 10-12 weeks of gestation, gene expression changes were already evident in chorionic villus samples from women subsequently diagnosed with preeclampsia. Histological comparison of placentas from normal and preeclamptic pregnancies showed preeclampsia-associated defects in spiral artery remodeling and trophoblast invasion, causing placental hypoperfusion, impaired placentation, placental hypoxia and ischemia. While phenotyping or genotyping the placenta early in gestation is feasible through chorionic villous sampling, this approach is invasive and associated with a significant risk for pregnancy loss, greatly precluding its application as a screening tool.

Therefore, there is still a need for a robust and non-invasive method for the presymptomatic detection of preeclampsia.

### SUMMARY

The present invention relates to a method for the prediction of the risk of developing preeclampsia in a pregnant human subject, wherein said subject's gestational age is under 140 days, comprising the steps of:
a. measuring in a sample from said subject a plurality of loci-specific DNA methylation levels;
b. deriving from said measurement a DNA methylation profile for said subject;
c. comparing said DNA methylation profile to,
   i. a reference DNA methylation profile corresponding to a high risk of preeclampsia group; and/or,
   ii. a reference DNA methylation profile corresponding to a low risk of preeclampsia group; and,
d. assigning on the basis of said comparison said subject to a preeclampsia risk group, thereby predicting the risk of developing preeclampsia in said subject.

In one embodiment, said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 3472, as found in the human genome build Grch37/hg19.

In one embodiment, said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 1768, as found in the human genome build Grch37/hg19.

In one embodiment, said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 1026, as found in the human genome build Grch37/hg19.

In one embodiment, said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 652, as found in the human genome build Grch37/hg19.

In one embodiment, said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 610, as found in the human genome build Grch37/hg19.

In one embodiment, said measuring step comprises measuring a plurality of loci-specific DNA methylation levels in a set of genomic regions comprising 11 genomic regions wherein each of said 11 genomic regions is distinct and defined in reference to one of SEQ ID NO: 601 to 611, as found in the human genome build Grch37/hg19.

In one embodiment, said measuring step comprises measuring a plurality of loci-specific DNA methylation levels in a set of genomic regions comprising 12 genomic regions wherein each of said 12 genomic regions is distinct and defined in reference to one of SEQ ID NO: 601 to 612, as found in the human genome build Grch37/hg19.

In one embodiment, said measuring step comprises measuring a plurality of loci-specific DNA methylation levels in a set of genomic regions comprising 29 genomic regions wherein each of said 29 genomic regions is distinct and defined in reference to one of SEQ ID NO: 601 to 629, as found in the human genome build Grch37/hg19.

In one embodiment, said subject's gestational age is under 133 days, preferably under 126 days or under 119 days, more preferably under 112 days.

In one embodiment, said subject's gestational age is ranging from 28 days to 111 days, preferably is ranging from 63 days to 104 days.

In one embodiment, said reference DNA methylation profile corresponding to a high risk of preeclampsia group is derived from the measure of said plurality of loci-specific DNA methylation levels in control subjects, preferably gestational age-matched control subjects, that developed preeclampsia at later stage of pregnancy.

In one embodiment, said reference DNA methylation profile corresponding to a low risk of preeclampsia group is derived from the measure of said plurality of loci-specific DNA methylation levels in control subjects, preferably gestational age-matched control subjects, that remained healthy in respect to preeclampsia during their pregnancy.

In one embodiment, said sample is a blood sample.

In one embodiment, said DNA is cell-free DNA, preferably circulating cell-free DNA. In one embodiment said DNA is circulating cell-free DNA.

In one embodiment, said DNA methylation is methylation of cytosine, preferably methylation of cytosine in a CpG dinucleotide.

In one embodiment, said measuring step comprises measuring a plurality of loci-specific DNA methylation levels in at least one genomic DNA region, wherein said at least one genomic region is defined in reference to one of SEQ ID NO: 1 to 600, as found in the human genome build Grch37/hg19, preferably in reference to one of SEQ ID NO: 1 to 200, as found in the human genome build Grch37/hg19.

In one embodiment, said measuring step comprises measuring a plurality of loci-specific DNA methylation levels in at least one genomic DNA region, wherein said at least one genomic DNA region consists of a sequence having at least 95% sequence identity with a sequence selected from the group consisting of SEQ ID NO: 1 to 600, preferably selected from the group consisting of SEQ ID NO: 1 to 200.

The present invention also relates to a kit comprising a set of capture probes specific for at least 2 genomic DNA regions, wherein each of said at least two genomic regions are distinct and defined in reference to one of SEQ ID NO: 1 to 600, as found in the human genome build Grch37/hg19, preferably in reference to one of SEQ ID NO: 1 to 200, as found in the human genome build Grch37/hg19.

In one embodiment, said kit comprising a set of capture probes specific for at least 10 genomic regions, wherein each of said at least 10 genomic regions are distinct and defined in reference to one of SEQ ID NO: 601 to 3472, as found in the human 5 genome build Grch37/hg19.

In one embodiment, said kit comprising a set of capture probes specific for at least 10 genomic regions, wherein each of said at least 10 genomic regions are distinct and defined in reference to one of SEQ ID NO: 601 to 610, as found in the human 10 genome build Grch37/hg19.

The present invention also relates to the use of the kit of the invention for the prediction of the risk of developing preeclampsia in a human subject.

### Definitions

In the present invention, the following terms have the following meaning.

As used herein, the term “DNA methylation” refers to covalent attachment of a methyl or hydroxymethyl group on one more nucleotide present in a deoxyribonucleic acid (DNA) molecule. The term includes, without being limited to, methylation of cytosine, the covalent attachment of a methyl or hydroxymethyl group at the position 5 of a cytosine's pyrimidine ring thereby forming 5-methylcytosine or 5-hydromethylcytosine. Methylation of cytosine is almost exclusively found in the DNA sequence context of a cytosine nucleotide is followed by a guanine nucleotide, referred herein as a CpG or a CG dinucleotide.

As used herein, the term “identity”, when used in a relationship between the sequences of two or more polypeptides or of two or more nucleic acid sequences, refers to the degree of sequence relatedness between polypeptides or nucleic acid sequences (respectively), as determined by the number of matches between strings of two or more amino acid residues or of two or more nucleotides, respectively. “Identity” measures the percent of identical matches between the smaller of two or more sequences with gap alignments (if any) addressed by a particular mathematical model or computer program (i.e., “algorithms”). Identity of related polypeptides or NA sequences can be readily calculated by known methods. Such methods include, but are not limited to, those described in Arthur M. Lesk, Computational Molecular Biology: Sources and Methods for Sequence Analysis (New-York: Oxford University Press, 1988); Douglas W. Smith, Biocomputing: Informatics and Genome Projects (New-York: Academic Press, 1993); Hugh G. Griffin and Annette M. Griffin, Computer Analysis of Sequence Data, Part 1 (New Jersey: Humana Press, 1994); Gunnar von Heinje, Sequence Analysis in Molecular Biology: Treasure Trove or Trivial Pursuit (Academic Press, 1987); Michael Gribskov and John Devereux, Sequence Analysis Primer (New York: M. Stockton Press, 1991); and Carillo et al., 1988. SIAM J. Appl. Math. 48(5): 1073-1082. Preferred methods for determining identity are designed to give the largest match between the sequences tested. Methods of determining identity are described in publicly available computer programs. Preferred computer program methods for determining identity between two sequences include the GCG program package, including GAP (Devereux et al., 1984. Nucl. Acid. Res. 12(1 Pt 1):387-395; Genetics Computer Group, University of Wisconsin Biotechnology Center, Madison, WI), BLASTP, BLASTN, TBLASTN and FASTA (Altschul et al., 1990. J. Mol. Biol. 215(3): 403-410). The BLASTX program is publicly available from the National Center for Biotechnology Information (NCBI) and other sources (BLAST Manual, Altschul et al. NCB/NLM/NIH Bethesda, Md. 20894; Altschul et al., 1990. J. Mol. Biol. 215(3): 403 410). The well-known Smith Waterman algorithm may also be used to determine identity.

As used herein, the term “gestational age” refers to the measure of the age, at the time of sample collection, of a subject's pregnancy which is taken from the beginning of the subject's last menstrual period (day 0). When expressed in week herein, the correspondence between weeks and days is as follows, week 0 correspond to a gestational age ranging 0 to 6 days, week 1 corresponds to a gestational age of 7 days to 13 days. The correspondence increment following the same progression. The skilled artisan is familiar with techniques to evaluate a subject's, a human subject in the context of the invention, gestational age. Such techniques include, without being limited to, direct calculation from the known first day of the last menstruation period, obstetric ultrasound, adding 14 days to a known duration following fertilization (useful for instance in the context of in vitro fertilization).

As used herein, the term “preeclampsia” refers to the multisystem disorder of pregnancy, also known as pre-eclampsia or preeclampsia toxaemia and characterized by new onset hypertension and often proteinuria in the second half of pregnancy. The term includes both early-onset preeclampsia and late-onset preeclampsia, depending on whether the diagnosis is made before week 34 of pregnancy or after. In one embodiment, preeclampsia is early onset-preeclampsia or late-onset preeclampsia. In one embodiment, preeclampsia is early-onset preeclampsia. In one embodiment, preeclampsia is late-onset preeclampsia.

### DETAILED DESCRIPTION

Here, the inventors have found that the risk of developing preeclampsia is associated with variations of the DNA methylation level at several positions within a sample of genomic DNA from a pregnant subject and that said variations are detectable before the onset of preeclampsia symptoms, in particular in the first trimester of pregnancy. The inventors have hence developed methods for the presymptomatic prediction of the risk of developing preeclampsia based on the measure of loci-specific DNA methylation levels. They have found that such preeclampsia-associated changes in the DNA methylation profile are detectable by measuring loci-specific DNA methylation levels of cell free DNA found in non-invasively accessible blood plasma samples. The method of the invention thus allows for the robust presymptomatic prediction of the risk of developing preeclampsia, and it may be implemented on samples that may be obtained using non-invasive sampling.

The present invention relates to a method for the prediction of the risk of developing preeclampsia in a human subject.

In one embodiment, the method of the invention is for the stratification of a subject for her preeclampsia risk, for the evaluation of the risk of developing preeclampsia in a subject, for the prognosis of preeclampsia in a subject and/or for the prediction of the risk of developing preeclampsia in a subject.

In one embodiment, the method of the invention is used before the onset or appearance of at least one, preferably at least two, symptom of preeclampsia in the subject. In one embodiment, the symptom of preeclampsia is a new-onset symptom.

Examples of symptoms of preeclampsia include, without being limited to, hypertension, proteinuria, uteroplacental dysfunction, thrombocytopenia, renal insufficiency, elevated liver transaminases, pulmonary edema and neurological complications, such as for example eclampsia, altered mental status, severe headaches and persistent visual scotomata.

In one embodiment, the method of the invention is used before the onset or appearance of hypertension and at least one other symptoms of preeclampsia in the subject. In one embodiment, the method of the invention is used before the onset of hypertension and at least one other symptoms of preeclampsia in the subject, wherein said at least one other symptoms of preeclampsia is selected from the group consisting of proteinuria, uteroplacental dysfunction, thrombocytopenia, renal insufficiency, elevated liver transaminases, pulmonary edema, eclampsia, altered mental status, severe headaches and persistent visual scotomata.

In one embodiment, the method of the invention is for the presymptomatic stratification of a pregnant subject for her preeclampsia risk, for the presymptomatic evaluation of the risk of developing preeclampsia in a subject, for the presymptomatic prognosis of preeclampsia in a subject and/or for the presymptomatic prediction of the risk of developing preeclampsia in a subject.

In one embodiment, the method of the invention is for the presymptomatic prediction of the risk of developing preeclampsia in a subject.

In the context of the invention, the subject is human. In one embodiment, the subject is pregnant or has presumptive signs of pregnancy.

In one embodiment, the subject is pregnant with twins. In one embodiment, the subject had preeclampsia in a previous pregnancy. In one embodiment, the subject is pregnant with a single embryo. In one embodiment, the subject has no history of preeclampsia.

In one embodiment, the subject is substantially healthy in respect to preeclampsia. In one embodiment, the subject is does not have at least one, preferably at least two, preeclampsia symptoms. In one embodiment, the subject does not have at least one of the symptoms, preferably new-onset symptom, of preeclampsia, wherein said at least one symptom of preeclampsia is selected from the group consisting of hypertension, proteinuria, uteroplacental dysfunction, thrombocytopenia, renal insufficiency, elevated liver transaminases, pulmonary edema, eclampsia, altered mental status, severe headaches and persistent visual scotomata. In one embodiment, the subject does not have hypertension, preferably new-onset hypertension, and at least one other symptoms, preferably new-onset symptom, of preeclampsia, wherein and said at least one other symptoms of preeclampsia is selected from the group consisting of proteinuria, uteroplacental dysfunction, thrombocytopenia, renal insufficiency, elevated liver transaminases, pulmonary edema, eclampsia, altered mental status, severe headaches and persistent visual scotomata.

In one embodiment, the subject's gestational age is under 240 days, preferably under 231 days, 224 days, 217 days, 210 days, 203 days, 196 days, 189 days, 182 days, 175 days, 168 days, 161 days, 154 days or under 147 days, more preferably under 140 days, 133 days, 126 days, 119 days or under 112 days, even more preferably under 105 days. In one embodiment, the subject's gestational age is under 133 days, preferably under 126 days or under 119 days more preferably under 112 days.

In one embodiment, the subject's gestational age is above 27 days, preferably above 34 days, 41 days, 48 days or above 55 days, more preferably above 62 days.

In one embodiment, the subject gestational age is ranging from 28 days to 237 days, preferably is ranging from 28 days to 223 days, from 28 days to 209 days, from 28 days to 195 days, from 28 days to 181 days, from 28 days to 167 days, from 28 days to 160, from 28 days to 153 days or from 28 days to 146 days, more preferably ranging from 28 days to 139 days, from 28 days to 132 days, from 28 days to 125 days, from 28 days to 118 days, from 28 days to 111 days or from 28 days to 104, even more preferably ranging from 35 days to 104 days, from 42 days to 104 days, from 49 days to 104 days, from 56 days to 104 days or from 63 days to 104 days. In one embodiment, the subject gestational age is ranging from 28 days to 111 days, preferably is ranging from 63 days to 104 days.

In one embodiment, the method of the invention is for the prediction, preferably presymptomatic prediction, of the risk of developing preeclampsia in a human, wherein said subject's gestational age is under 140 days.

In one embodiment, the method of the invention comprises a step of measuring in a sample from the subject at least one, or a plurality of, loci-specific DNA methylation level(s). In one embodiment, the method of the invention comprises a step of measuring in a sample from the subject a plurality of loci-specific DNA methylation level(s).

As used herein, the term loci-specific DNA methylation refers to DNA methylation of a nucleotide at a specific location in a DNA sequence. The term is defined in opposition to global DNA methylation that can be assayed without retaining information of the localization (or locus) of the nucleotide considered in the DNA sequence.

The skilled artisan is familiar with techniques allowing the measure of loci-specific DNA methylation level. Such techniques include, without being limited to, bisulfite sequencing, enzymatic methylome sequencing, TET-assisted pyridine borane sequencing, DNA immunoprecipitation, direct or native 5-methylcytosine or 5-hydroxymethylcytosine sequencing and the like.

In one embodiment, DNA methylation is methylation of cytosine, preferably methylation of cytosine in a CpG dinucleotide.

In one embodiment, said DNA is genomic DNA. As used herein, the term genomic DNA refers to the nuclear genome. In one embodiment, said DNA is cell-free DNA, preferably circulating cell-free DNA (cfDNA). As used herein, the term cell-free DNA refers to DNA found in the bodily fluids of the subject outside of said subject's cells. Examples of bodily fluid that may be considered in the context of the invention include, without being limited to, blood, plasma, serum, cervical smear and amniotic fluid. The term circulating cell-free DNA refers to cell-free DNA found in blood, plasma or serum. In one embodiment, said DNA is cell-free genomic DNA. In one embodiment, said DNA is human DNA. In one embodiment, said DNA is cell-free human genomic DNA.

In one embodiment, the method of the invention comprises a step of measuring in a sample from the subject at least one, or a plurality of, loci-specific DNA methylation level(s) in at least one, or in a set of, genomic region(s). It is to be understood that in the context of the invention, when a plurality of loci-specific DNA methylation levels are measured in a plurality of genomics regions, at least one loci-specific DNA methylation level is measured in each of said genomic regions.

As used herein, the terms genomic region or genomic DNA region refers to a portion or fragment of the genome, in the context of the invention, of the human nuclear genome. Genomic regions are identified or defined by the location in the genome of where they begin and end. The location of the begin and end points of a given genomic region may, for instance and without limitation, be identified or defined by their coordinates given as the combination of the identification of the reference of the genome sequence used, the chromosome number, if applicable, and the nucleotide coordinate on the reference strand (also known as the top strand, the Watson strand or the +strand) and with the base coordinate system used for the reference of the genome sequence used (e.g. human genome build Grch37/hg19; chr5:43,484,005-43,484,180 in the one-based coordinate system corresponds to a specific region of SEQ ID NO: 1 in the human genome). The location of the begin and end points of a given genomic region may also, for instance and without limitation, be identified by or defined by or defined in reference to the provision of its nucleic acid sequence. It is then within the reach of the skilled artisan provided with the nucleic acid sequence of a genomic region to locate its position within a given species' genome using sequence comparison tools such as for instance and without limitation, using BLAT (Kent, Genome Research 4: 656-664).

As used herein, the terms human genome build Grch37/hg19 is used in reference to the assembly available under the Genbank Assembly Accession number GCA_000001405.1.

In one embodiment, said genomic region comprises one or more CpG dinucleotide.

In one embodiment, the method of the invention comprises a step of measuring in a sample from the subject a plurality of loci-specific DNA methylation levels in at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably at least 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or at least 600 genomic regions.

In one embodiment, the method of the invention comprises a step of measuring in a sample from the subject at least one, or a plurality of, loci-specific DNA methylation level(s) in at least one genomic region, wherein said at least one genomic region is identified by sequence comparison as corresponding to, or defined by, or defined in reference to, one of SEQ ID NO: 1 to 600, as found in the human genome build Grch37/hg19, preferably is identified by sequence comparison as corresponding to, or defined by, or defined in reference to, one of SEQ ID NO: 1 to 200, as found in the human genome build Grch37/hg19.

In one embodiment, the method of the invention comprises a step of measuring in a sample from the subject at least one, or a plurality of, loci-specific DNA methylation level(s) in at least one genomic region, wherein said at least one genomic region consists of a sequence having at least 90%, 91%, 92%, 93% or 94%, preferably having at least 95%, 96%, 97%, or 98%, more preferably having at least 99% sequence identity with a sequence selected from the group consisting of SEQ ID NO: 1 to 600, preferably selected from the group consisting of SEQ ID NO: 1 to 200. In one embodiment, said at least one genomic region consists of a sequence selected from the group consisting of SEQ ID NO: 1 to 600. In one embodiment, said at least one genomic region consists of a sequence selected from the group consisting of SEQ ID NO: 1 to 200.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably comprises or consists of, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599 or 600 genomic regions, wherein each of said genomics regions is distinct and identified by sequence comparison as corresponding to, or defined by, or defined in reference to, one of SEQ ID NO: 1 to 600, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 or 11, more preferably comprises or consists of, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287 or 288 genomic regions, wherein each of said genomic regions is distinct and identified by sequence comparison as corresponding to, or defined by, or defined in reference to, one of SEQ ID NO: 601 to 3472, as found in the human genome build Grch37/hg19, preferably wherein each of said genomic regions is distinct and identified by sequence comparison as corresponding to, or defined by, or defined in reference to, one of SEQ ID NO: 601 to 1768, as found in the human genome build Grch37/hg19, more preferably wherein each of said genomic regions is distinct and identified by sequence comparison as corresponding to, or defined by, or defined in reference to, one of SEQ ID NO: 601 to 1026, as found in the human genome build Grch37/hg19.

In one embodiment, the size of said set of genomic regions is adjusted depending of the predictive value of the specific genomic regions included to achieve the desired specificity and sensitivity of the method of the invention. It is within the reach of the skilled artisan to adjust the content (number of sequence and choice of sequences) of the set of genomic regions, for instance and without limitation using the absolute coefficient as illustrated in the example provided in the present application that are indicative of the predictive value of a given genomic region.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 or 11, more preferably comprises or consists of, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 or 144 genomic regions, wherein each of said genomic regions is distinct and identified by sequence comparison as corresponding to, or defined by, or defined in reference to, one of SEQ ID NO: 601 to 744, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 or 11, more preferably comprises or consists of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 genomic regions, wherein each of said genomic regions is distinct and identified by sequence comparison as corresponding to, or defined by, or defined in reference to, one of SEQ ID NO: 601 to 652, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 genomic regions, wherein each of said genomic regions is distinct and identified by sequence comparison as corresponding to, or defined by, or defined in reference to, one of SEQ ID NO: 601 to 610, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9 or 10, preferably comprises or consists of 11 genomic regions, wherein each of said genomic regions is distinct and identified by sequence comparison as corresponding to, or defined by, or defined in reference to, one of SEQ ID NO: 601 to 611, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10 or 11, preferably comprises or consists of 12 genomic regions, wherein each of said genomic regions is distinct and identified by sequence comparison as corresponding to, or defined by, or defined in reference to, one of SEQ ID NO: 601 to 612, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13 or 14 preferably comprises or consists of 15 genomic regions, wherein each of said genomic regions is distinct and identified by sequence comparison as corresponding to, or defined by, or defined in reference to, one of SEQ ID NO: 601 to 615, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27 or 28, preferably comprises or consists of 29 genomic regions, wherein each of said genomic regions is distinct and identified by sequence comparison as corresponding to, or defined by, or defined in reference to, one of SEQ ID NO: 601 to 629, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably comprises or consists of, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599 or 600 genomic regions, wherein each of said genomics regions consists of, a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 600. In one embodiment, each of said genomics regions consists of one sequence selected from the group consisting of SEQ ID NO: 1 to 600.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, or 9, preferably comprises or consists of 10, 11, more preferably comprises or consists of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287 or 288 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 3472, preferably wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 1768, more preferably wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 1026.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 or 11, more preferably comprises or consists of, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 or 144 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 744.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 or 11, more preferably comprises or consists of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 652.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8 or 9, preferably comprises or consists of 10 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 610.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9 or 10, preferably comprises or consists of 11 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 611.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10 or 11, preferably comprises or consists of 12 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 612.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13 or 14 preferably comprises or consists of 15 genomic regions, wherein each of said genomic regions consists of a sequence having at least 90%, 91%, 92%, 93%, 94%, 95%, 96%, 97%, 98% or at least 99% sequence identity with one distinct sequence selected from the group consisting of SEQ ID NO: 601 to 615.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27 or 28, preferably comprises or consists of 29 genomic regions, wherein each of said genomic regions consists of, a sequence having at least 90%, 91%, 92%, 93%, 94%, 95%, 96%, 97%, 98% or at least 99% sequence identity with one distinct sequence selected from the group consisting of SEQ ID NO: 601 to 629.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably comprises, or consists of, 200 genomic regions, wherein each of said genomic regions is distinct and identified by sequence comparison as corresponding to, or defined by, or defined in reference to, one of SEQ ID NO: 1 to 200, as found in the human genome build Grch37/hg19.

In one embodiment, the method of the invention comprises a step of measuring in a sample from the subject a plurality of loci-specific DNA methylation levels in a set of genomic regions, wherein said set of genomic regions comprises, or consists of, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably comprises, or consists of, 200 genomic regions, wherein each of said genomics region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 200. In one embodiment, each of said genomics regions consists of one sequence selected from the group consisting of SEQ ID NO: 1 to 200.

In one embodiment, the method of the invention comprises a step of deriving from the measure in a sample from the subject at least one, or a plurality of, loci-specific DNA methylation level(s), a DNA methylation profile for the subject. In one embodiment, the method of the invention comprises a step of deriving from the measure in a sample from the subject of at least one, or a plurality of loci-specific DNA methylation levels, a DNA methylation profile for the subject.

As used herein, the term DNA methylation profile refers to a set of data representing the level of DNA methylation of one or more loci for a given subject in a form suitable for comparison purposes. It is within the reach of the skilled artisan to select the most appropriate data set representation, such as for example and without limitation, raw values, means, medians, or any form of mathematical or graphical representation. The profile may indicate the methylation level of every loci in a subject, can have information regarding a subset of the loci in a genome, or can have information regarding regional methylation density. When deriving methylation profile for a subject it is for example and without limitation, possible to define the methylation level of a given region of interest as the ratio of methylated position (for example and without limitation, methylated cytosine or methylated cytosine in a CpG context) relative to the total of position that can be methylated in said region of interest (for example, and without limitation, the total number of cytosine, or cytosine in a CpG context, in said region of interest). When deriving the methylation profile for a subject is may also be possible to select the measurements to be included in the profile, for instance, and without limitation, it is possible to select the measurements based on the quality of the measure and/or their information content as illustrated in the example section.

In one embodiment, the method of the invention comprises a step of comparing the methylation profile of the subject to one or more reference methylation profile(s).

As used herein the term reference DNA methylation profile refers to a DNA methylation profile for a given control subject, or for a given population of control subjects, of known outcome in respect to the development of preeclampsia. The terms “control subject” is used herein to refer to a subject of know outcome in respect to preeclampsia that is used to derive a reference DNA methylation profile. It is within the reach of the skilled artisan to select for the reference DNA methylation profile the most appropriate data set representation, such as for example and without limitation, raw values, means, medians, or any form of mathematical or graphical representation. When deriving the reference methylation profile for a control subject or, preferably for a population of control subjects, it is for example, and without limitation, possible to use the level of DNA methylation of one or more loci for a given control subject, or for a given population of control subjects, of known outcome in respect to the development of preeclampsia to build a graphical representation and/or a mathematical model of said control subject or population of control subjects.

Such a model may be for instance, and without limitation, a hierarchical clustering analysis, a generalized linear or logistic model, a LASSO model, an elastic net regularized generalized linear or logistic model, a Tikhonov regularization model and the like. It is to be understood in the context of the comparison step of the invention, that the reference DNA methylation profile comprise information on the level of methylation of the same one or more loci that are considered in the measuring step. Therefore, embodiment relating to the loci or plurality thereof considered in the measuring step may apply to the determination of the reference DNA methylation profile.

In one embodiment, the reference DNA methylation profile corresponds to a risk of preeclampsia group. It is to be understood in the context of the invention that risk of preeclampsia group is used in respect to a reference DNA methylation profile to indicate that, the similarity, or dissimilarity, to said reference DNA methylation is indicative of the level of risk, such as for instance and without limitation, the probability, of developing preeclampsia at later stage of pregnancy. It is within the reach of the skilled artisan to define the risk groups, depending on parameters such as for instance, and without limitation, the targeted sensitivity or specificity or the number of groups to be defined.

In one embodiment, the reference DNA methylation profile corresponds to a high risk of preeclampsia group. In one embodiment, said high risk of developing preeclampsia correspond to a probability of developing preeclampsia above the probability corresponding to the incidence of preeclampsia of the population, preferably of the population relevant to the tested subject considered. In one embodiment, said high risk of developing preeclampsia correspond to a probability of developing preeclampsia above 0.04, preferably above 0.05, 0.06, 0.07, 0.08, 0.09, 0.1, 0.11, 0.12, 0.13, 0.14, 0.2, 0.3, 0.4, 0.5, 0.6, 0.7, 0.8, 0.9 or above.

In one embodiment, the reference DNA methylation profile corresponds to a low risk of preeclampsia group. In one embodiment, said low risk of developing preeclampsia correspond to a probability of developing preeclampsia below one minus the probability corresponding to the incidence of preeclampsia of the population, preferably of the population relevant to the tested subject considered. In one embodiment, said low risk of developing preeclampsia correspond to a probability of developing preeclampsia below 0.96, preferably below 0.95, 0.94, 0.93, 0.92, 0.91, 0.9, 0.89, 0.88 0.87, 0.86, 0.8, 0.7, 0.6, 0.5, 0.4, 0.3, 0.2, 0.1 or below.

In one embodiment, the reference DNA methylation profile is derived from the measure of a plurality of loci-specific DNA methylation levels in control subjects that developed preeclampsia at later stage of pregnancy. In one embodiment, the reference DNA methylation profile corresponding to a high risk of preeclampsia group is derived from the measure of a plurality of loci-specific DNA methylation levels in control subjects that developed preeclampsia at later stage of pregnancy.

In one embodiment, the reference DNA methylation profile is derived from the measure of a plurality of loci-specific DNA methylation levels in control subjects that remained healthy in respect to preeclampsia during their pregnancy. In one embodiment, the reference DNA methylation profile corresponding to a low risk of preeclampsia group is derived from the measure of a plurality of loci-specific DNA methylation levels in control subjects that remained healthy in respect to preeclampsia during their pregnancy.

It is within the reach of the skilled artisan to select the control subject(s) to be considered in order to determine a reference DNA methylation profile. For instance, and without limitation, the skilled artisan is able to account for factors influencing the risk of developing preeclampsia such as for instance Body Mass Index, parity and the like. Likewise, the skilled artisan is able to select the appropriate gestational age at the time of sampling the control subject to be included in the reference, matching that of the subject whose methylation profile will be compared to the reference methylation profile (age-matched control subject(s)).

In one embodiment, the reference DNA methylation profile is derived from the measure of a plurality of loci-specific DNA methylation levels in aged-matched control subjects that developed preeclampsia at later stage of pregnancy. In one embodiment, the reference DNA methylation profile corresponding to a high risk of preeclampsia group is derived from the measure of a plurality of loci-specific DNA methylation levels in aged-matched control subjects that developed preeclampsia at later stage of pregnancy.

In one embodiment, the reference DNA methylation profile is derived from the measure of a plurality of loci-specific DNA methylation levels in aged-matched control subjects that remained healthy in respect to preeclampsia during their pregnancy. In one embodiment, the reference DNA methylation profile corresponding to a low risk of preeclampsia group is derived from the measure of a plurality of loci-specific DNA methylation levels in aged-matched control subjects that remained healthy in respect to preeclampsia during their pregnancy.

In one embodiment, the method of the invention comprises a step of comparing the methylation profile of the subject to,
i. a reference DNA methylation profile corresponding to a high risk of preeclampsia group; and/or,
ii. a reference DNA methylation profile corresponding to a low risk of preeclampsia group.

In one embodiment, the method of the invention comprises a step of comparing the methylation profile of the subject to,
i. a reference DNA methylation profile corresponding to a high risk of preeclampsia group, wherein said reference DNA methylation profile corresponding to a high risk of preeclampsia group is derived from the measure of the plurality of loci-specific DNA methylation levels in control subjects, preferably gestational age-matched control subjects, that developed preeclampsia at later stage of pregnancy; and/or,
ii. a reference DNA methylation profile corresponding to a low risk of preeclampsia group, wherein said reference DNA methylation profile corresponding to a low risk of preeclampsia group is derived from the measure of the plurality of loci-specific DNA methylation levels in control subjects, preferably gestational age-matched control subjects, that remained healthy in respect to preeclampsia during their pregnancy.

In one embodiment, the method of the invention comprises a step of assigning the subject to a preeclampsia risk group. The assignment results in, the stratification of the subject for her preeclampsia risk, in the evaluation of the risk of developing preeclampsia in the subject, in the prognosis of preeclampsia in the subject and/or in the prediction of the risk of developing preeclampsia in the subject. In one embodiment, the method of the invention comprises a step of assigning the subject to a preeclampsia risk group, thereby predicting the risk of developing preeclampsia in said subject

In one embodiment, the DNA methylation profile of the subject is not different from, preferably not statistically different from, a reference DNA methylation profile corresponding to a high risk of preeclampsia group and said subject is assigned to said high risk of preeclampsia group and/or the DNA methylation profile is different from, preferably statistically different from, a reference DNA methylation profile corresponding to low risk of preeclampsia group(s) and the subject is assigned to a high risk of preeclampsia group.

In one embodiment, the DNA methylation profile of the subject is not different from, preferably not statistically different from, a reference DNA methylation profile corresponding to a low risk of preeclampsia group and said subject is assigned to said low risk of preeclampsia group and/or the DNA methylation profile is different from, preferably statistically different from, a reference DNA methylation profile corresponding to high risk of preeclampsia group(s) and the subject is assigned to a low risk of preeclampsia group.

In the context of the invention, difference between the subject DNA methylation profile and the reference methylation profile arise from the lower methylation level (hypomethylation) or higher (hypermethylation) of certain loci and/or region in the subject when compared with the reference methylation profile.

In one embodiment, the method of the invention comprises a step of providing a sample from the subject.

In one embodiment, the sample was previously taken form the subject. The method of the invention does not comprise a step of collecting the sample from the subject. in this embodiment, the method of the invention is an in vitro method.

In one embodiment, the sample is a tissue sample or a bodily fluid.

Examples of bodily fluid that may be considered in the context of the invention include, without being limited to, blood, plasma, serum, urine, cervical smears, fluids and aspirates and amniotic fluid.

Example of tissue sample that may be considered in the context of the invention include, without being limited to chorionic villus biopsy, cervical biopsy, fetus-derived tissue biopsy and endometrial tissue.

In one embodiment the sample is a bodily fluid. In one embodiment, the sample is a blood sample, a serum sample, a plasma sample or an amniotic fluid sample. In one embodiment, the sample is a blood, serum or plasma sample. In one embodiment, the sample is maternal or uteroplacental blood, serum or plasma sample. In one embodiment, the sample is a blood sample, preferably a plasma sample. In one embodiment, the sample is a maternal or uteroplacental blood sample, preferably a maternal or uteroplacental plasma sample.

In one embodiment, the sample a contain cell-free DNA. In one embodiment, the sample is a bodily fluid and contains cell-free DNA. In one embodiment, the sample is blood, plasma or serum sample and contain circulating cell-free DNA (cfDNA). In one embodiment, the sample is maternal or uteroplacental blood, serum or plasma sample and contains circulating cell-free DNA (cfDNA).

It is to be understood that embodiment contained in the application may be combined in the methods of the invention.

In one embodiment, the method of the invention is a method for the prediction, preferably presymptomatic prediction, of the risk of developing preeclampsia in a human subject comprising the steps of:
a. measuring in a sample from said subject at least one, or a plurality of, loci-specific DNA methylation level(s);
b. deriving from said measurement a DNA methylation profile for said subject;
c. comparing said DNA methylation profile to at least one reference DNA methylation profile corresponding to a risk group; and,
d. assigning on the basis of said comparison said subject to said at least one preeclampsia risk group, thereby predicting the risk of developing preeclampsia in said subject.

In one embodiment, the method of the invention is a method for the prediction, preferably presymptomatic prediction, of the risk of developing preeclampsia in a human subject, wherein said subject's gestational age is under 140 days, comprising the steps of:
a. measuring in a sample from said subject at least one, or a plurality of, loci-specific DNA methylation levels;
b. deriving from said measurement a DNA methylation profile for said subject;
c. comparing said DNA methylation profile to at least one reference DNA methylation profile corresponding to a risk group; and,
d. assigning on the basis of said comparison said subject to said at least one preeclampsia risk group, thereby predicting the risk of developing preeclampsia in said subject.

In one embodiment, the method of the invention is a method for the prediction, preferably presymptomatic prediction, of the risk of developing preeclampsia in a human subject, wherein said subject's gestational age is under 140 days, comprising the steps of:
a. measuring in a sample from said subject a plurality of loci-specific DNA methylation levels;
b. deriving from said measurement a DNA methylation profile for said subject;
c. comparing said DNA methylation profile to,
   i. a reference DNA methylation profile corresponding to a high risk of preeclampsia group; and/or,
   ii. a reference DNA methylation profile corresponding to a low risk of preeclampsia group; and,
d. assigning on the basis of said comparison said subject to a preeclampsia risk group, thereby predicting the risk of developing preeclampsia in said subject.

In one embodiment, the method of the invention further comprises a step of extracting DNA from the sample, preferably extracting genomic DNA from the sample. This step is performed of before the step of measuring in a sample from said subject a plurality of loci-specific DNA methylation levels. It is within the reach of the skilled artisan to select the appropriate DNA extraction methods depending, for example and without limitation on the nature of the sample and/or the subsequent method used to measure the plurality of loci-specific DNA methylation levels.

In one embodiment, the method of the invention further comprises a step of performing target enrichment of one or a plurality of specific genomic region. This step is performed before the step of measuring in a sample from said subject a plurality of loci-specific DNA methylation levels and after the step of extracting DNA from the sample. The target enrichment step may be useful for instance when the measuring step is directed to specific genomic regions. The skilled artisan is familiar with techniques allowing target enrichment in a DNA sample. Target enrichment may be for instance, and without limitation, be implemented using capture probes as described in the example section.

The present invention also relates to a kit for implementing the method of the invention.

In one embodiment, the kit of the invention comprises a set of capture probe.

As used herein, the term capture probes correspond to oligonucleotides that hybridize specifically with a specific genomic region (whether or not it is bisulfite-converted), and that are modified to perform target enrichment on a genomic DNA sample and that may be used may be used to implement the method of the invention. Example of modification include, for instance and without limitation, the addition of a binding site on the oligonucleotide allowing the direct or indirect capture on a solid substrate of the oligonucleotide hybridized to a specific genomic DNA region. Such modification may be for instance, and without limitation, be implemented and used as described in the example section. With the knowledge of the DNA sequence of the targeted genomic region, it is within the reach of the skilled artisan to design capture probes.

In one embodiment, the kit of the invention comprise a set of capture probe, wherein said set of capture probe consist of capture probes specific for at least 2 genomic DNA regions, wherein each of said at least two genomic regions is distinct and identified by sequence comparison as corresponding to, or defined by, or defined in reference to, one of SEQ ID NO: 1 to 600, as found in the human genome build Grch37/hg19, preferably identified by sequence comparison as corresponding to, or defined by, or defined in reference to, one of SEQ ID NO: 1 to 200, as found in the human genome build Grch37/hg19.

In one embodiment, the kit of the invention comprise a set of capture probes, wherein said set of capture probes consist of capture probes specific for at least 10 genomic regions, wherein each of said at least 10 genomic regions is distinct and identified by sequence comparison as corresponding to, or defined by, or defined in reference to, one of SEQ ID NO: 601 to 3472, as found in the human genome build Grch37/hg19, preferably identified by sequence comparison as corresponding to, or defined by, or defined in reference to, one of SEQ ID NO: 601 to 610, as found in the human genome build Grch37/hg19.

In one embodiment, the kit of the invention comprise a set of capture probe, wherein said set of capture probe consist of capture probes specific for at least two genomic DNA regions wherein each of said at least two genomics regions consists of, a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 600. In one embodiment, each of said at least two genomics regions consists of one sequence selected from the group consisting of SEQ ID NO: 1 to 600.

In one embodiment, the kit of the invention comprise a set of capture probes, wherein said set of capture probes consist of capture probes specific for at least 10 genomic regions wherein each of said at least 10 genomics regions consists of, a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 3472. In one embodiment, each of said at least 10 genomics regions consists of one sequence selected from the group consisting of SEQ ID NO: 601 to 610.

In one embodiment, the kit of the invention comprise a set of capture probe, wherein said set of capture probe consists of capture probes specific for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 genomic regions wherein each of said genomic regions is distinct and identified by sequence comparison as corresponding to, or defined by, or defined in reference to, one of SEQ ID NO: 1 to 600, as found in the human genome build Grch37/hg19.

In one embodiment, the kit of the invention comprise a set of capture probes, wherein said set of capture probe consists of capture probes specific for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287 or 288 genomic regions wherein each of said genomic regions is distinct and identified by sequence comparison as corresponding to, or defined by, or defined in reference to, one of SEQ ID NO: 1 to 3472, as found in the human genome build Grch37/hg19. In one embodiment, each of said genomic regions is distinct and identified by sequence comparison as corresponding to, or defined by, or defined in reference to, one of SEQ ID NO: 1 to 1768, as found in the human genome build Grch37/hg19. In one embodiment, each of said genomic regions is distinct and identified by sequence comparison as corresponding to, or defined by, or defined in reference to, one of SEQ ID NO: 1 to 1026, as found in the human genome build Grch37/hg19.

In one embodiment, the kit of the invention comprise a set of capture probe, wherein said set of capture probe consists of capture probes specific for 10 genomic regions wherein each of said genomic regions is distinct and identified by sequence comparison as corresponding to, or defined by, or defined in reference to, one of SEQ ID NO: 601 to 610, as found in the human genome build Grch37/hg19.

In one embodiment, the kit of the invention comprise a set of capture probe, wherein said set of capture probe consists of capture probes specific for 11 genomic regions wherein each of said genomic regions is distinct and identified by sequence comparison as corresponding to, or defined by, or defined in reference to, one of SEQ ID NO: 601 to 611, as found in the human genome build Grch37/hg19.

In one embodiment, the kit of the invention comprise a set of capture probe, wherein said set of capture probe consists of capture probes specific for 12 genomic regions wherein each of said genomic regions is distinct and identified by sequence comparison as corresponding to, or defined by, or defined in reference to, one of SEQ ID NO: 601 to 612, as found in the human genome build Grch37/hg19.

In one embodiment, the kit of the invention comprise a set of capture probe, wherein said set of capture probe consists of capture probes specific for 13 genomic regions wherein each of said genomic regions is distinct and identified by sequence comparison as corresponding to, or defined by, or defined in reference to, one of SEQ ID NO: 601 to 613, as found in the human genome build Grch37/hg19.

In one embodiment, the kit of the invention comprise a set of capture probe, wherein said set of capture probe consists of capture probes specific for 14 genomic regions wherein each of said genomic regions is distinct and identified by sequence comparison as corresponding to, or defined by, or defined in reference to, one of SEQ ID NO: 601 to 614, as found in the human genome build Grch37/hg19.

In one embodiment, the kit of the invention comprise a set of capture probe, wherein said set of capture probe consists of capture probes specific for 15 genomic regions wherein each of said genomic regions is distinct and identified by sequence comparison as corresponding to, or defined by, or defined in reference to, one of SEQ ID NO: 601 to 615, as found in the human genome build Grch37/hg19.

In one embodiment, the kit of the invention comprise a set of capture probe, wherein said set of capture probe consists of capture probes specific for 29 genomic regions wherein each of said genomic regions is distinct and identified by sequence comparison as corresponding to, or defined by, or defined in reference to, one of SEQ ID NO: 601 to 629, as found in the human genome build Grch37/hg19.

In one embodiment, the kit of the invention comprise a set of capture probe, wherein said set of capture probe consists of capture probes specific for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 200 genomic regions, wherein each of said genomic regions is distinct and identified by sequence comparison as corresponding to, or defined by, or defined in reference to, one of SEQ ID NO: 1 to 200, as found in the human genome build Grch37/hg19.

In one embodiment, the kit of the invention comprise a set of capture probe, wherein said set of capture probe consists of capture probes specific for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 genomic regions, wherein each of said genomic region consists of, a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 600. In one embodiment, each of said at least two genomics regions consists of one sequence selected from the group consisting of SEQ ID NO: 1 to 600.

In one embodiment, the kit of the invention comprise a set of capture probe, wherein said set of capture probe consists of capture probes specific for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199, preferably 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287 or 288 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 3472. In one embodiment, each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 1768. In one embodiment, each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 1026.

In one embodiment, the kit of the invention comprise a set of capture probe, wherein said set of capture probe consists of capture probes specific for 10 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 610.

In one embodiment, the kit of the invention comprise a set of capture probe, wherein said set of capture probe consists of capture probes specific for 11 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 611.

In one embodiment, the kit of the invention comprise a set of capture probe, wherein said set of capture probe consists of capture probes specific for 12 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 612.

In one embodiment, the kit of the invention comprise a set of capture probe, wherein said set of capture probe consists of capture probes specific for 13 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 613.

In one embodiment, the kit of the invention comprise a set of capture probe, wherein said set of capture probe consists of capture probes specific for 14 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 614.

In one embodiment, the kit of the invention comprise a set of capture probe, wherein said set of capture probe consists of capture probes specific for 15 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 615.

In one embodiment, the kit of the invention comprise a set of capture probe, wherein said set of capture probe consists of capture probes specific for 29 genomic regions wherein each of said genomic region consists of a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 601 to 629.

In one embodiment, the kit of the invention comprise a set of capture probe, wherein said set of capture probe consists of capture probes specific for 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 genomic regions, wherein each of said genomic region consists of, a sequence having at least 90%, 91%, 92%, 93% or 94%, preferably having at least 95%, 96%, 97%, or 98%, more preferably having at least 99% sequence identity with one distinct sequence selected from the group consisting of SEQ ID NO: 1 to 200. In one embodiment, each of said genomics regions consists of one sequence selected from the group consisting of SEQ ID NO: 1 to 200.

The present invention also relates to the use of the kit of the invention for the stratification, preferably presymptomatic stratification, of a pregnant subject for her preeclampsia risk, for the evaluation, preferably presymptomatic evaluation, of the risk of developing preeclampsia in a subject, for the prognosis, preferably presymptomatic prognosis, of preeclampsia in a subject and/or for the prediction, preferably presymptomatic prediction, of the risk of developing preeclampsia in a subject. In one embodiment, the present invention relates to the use of the kit of the invention for the prediction of the risk of developing preeclampsia in a human subject.

In one embodiment, the present invention relates to the use of a kit for the presymptomatic prediction of the risk of developing preeclampsia in a human subject, wherein said kit comprises a set of capture probe specific for at least 2 genomic DNA regions and wherein each of said at least two genomic regions is distinct and identified by sequence comparison as corresponding to, or defined by, or defined in reference to, one of SEQ ID NO: 1 to 600, as found in the human genome build Grch37/hg19, preferably identified by sequence comparison as corresponding to, or defined by, or defined in reference to, one of SEQ ID NO: 1 to 200, as found in the human genome build Grch37/hg19.

The methods and kits of the invention may be used during the first trimester of pregnancy. As such, it is possible to use the method of the invention in combination without other test realized during the first trimester of pregnancy. Example of such test include, without being limited to, the non-invasive prenatal tests (NIPT), such as the Harmony prenatal test, CentoNIPT, percept NIPT, Vanadis NIPT, VERACITY and others.

The present invention further relates to methods for preventing preeclampsia in a pregnant human subject comprising the steps of:
a. determining the risk of developing preeclampsia when said subject's gestational age is under 140 days using the method of the invention for the prediction of the risk of developing preeclampsia, and,
b. administering to subject identified at step (a) as being at risk of developing preeclampsia, an effective dose of a treatment preventing the development of preeclampsia.

In one embodiment, said treatment preventing the development of preeclampsia comprises aspirin.

### BRIEF DESCRIPTION OF THE DRAWINGS

FIG. 1 is a receiver operating characteristic curve of 500 iterations of a 10-fold cross-validation elastic net analysis, trained on 80% of the DNA methylation measures for the 200 regions showing the most significant differences in DNA methylation between control and preeclamptic placentas and validated on 20% of the correctly labelled data (dashed line), or the same data after label randomization (full line). (value±SEM).

FIG. 2 is a whisker plot of the DNA concentrations of bisulfite sequencing libraries prepared from cfDNA samples derived from matched controls (control, n=9) and preeclamptic pregnancies (cases, n=21) and obtained at the time of preeclampsia diagnosis. P: significance calculated using a Wilcoxon test.

FIG. 3 is a receiver operating characteristic curve of 500 iterations of a 10-fold cross-validation elastic net analysis, trained on 80% of the DNA methylation measures for the 200 regions showing the most significant differences in DNA methylation between cfDNA samples from control and preeclamptic pregnancies obtained at the time of preeclampsia diagnosis and validated on 20% of the correctly labelled data (dashed line), or the same data after label randomization (full line). (value±SEM).

FIG. 4 is a receiver operating characteristic curve of 500 iterations of a 10-fold cross-validation elastic net analysis, trained on 80% of the DNA methylation measures for the 200 regions showing the most significant differences in DNA methylation cfDNA samples from control and preeclamptic pregnancies obtained at around 12 weeks of gestation and validated on 20% of the correctly labelled data (dashed line), or the same data after label randomization (full line). (value±SEM).

FIG. 5 is a set of receiver operating characteristic curve of 20 iterations of a 10-fold cross-validation elastic net analysis, trained on 1436 (FIG. 5A), 1149 (FIG. 5B), 862 (FIG. 5C), 288 (FIG. 5D), 29 (FIG. 5E), 15 (FIG. 5F) and 12 (FIG. 5G) randomly selected genomic regions from 2872 genomic regions (SEQ ID NO. 601 to SEQ ID 3472). AUC: Area Under Curve.

FIG. 6 is a set of set of receiver operating characteristic curve of 20 iterations of a 10-fold cross-validation elastic net analysis, trained on the 1436 (FIG. 6A), 1149 (FIG. 6B), 862 (FIG. 6C), 288 (FIG. 6D), 29 (FIG. 6E), 15 (FIG. 6F), 12 (FIG. 6G), 11 (FIG. 6H), 10 (FIG. 6I) and 9 (FIG. 6J) genomic regions with the highest absolute coefficient. AUC: Area Under Curve.

### EXAMPLES

The present invention is further illustrated by the following examples.

### Example 1: Analysis of Placental DNA Methylation Profile after Delivery

### Materials and Methods

### Patient Enrolment

Patients affected with preeclampsia and gestational age-matched controls were selected and enrolled in this study. Preeclampsia was defined as a new-onset hypertension (systolic blood pressure ≥140 mmHg or diastolic blood pressure ≥90 mmHg on at least two occasions at least four hours apart, or a systolic blood pressure ≥160 mmHg on a single occasion) and the coexistence of one or more of the following new-onset condition: proteinuria (≥0.3 g in a 24-hour urine specimen or protein/creatinine ratio ≥0.3 (mg/mg) in a random urine specimen), uteroplacental dysfunction (such as fetal growth restriction, abnormal umbilical artery doppler waveform analysis or stillbirth), thrombocytopenia (platelet count <100,000/microL), renal insufficiency (serum creatinine ≥1.1 mg/dl or doubling of serum creatinine in the absence of other renal disease), elevated liver transaminases (twice normal concentration), pulmonary oedema, or neurological complications (such as eclampsia, altered mental status, severe headaches or persistent visual scotomata) between 20 and 34 weeks of gestation. Control patients were matched for significant preeclampsia risk factors, including age, BMI and parity. The study was approved by the Medical Ethics Committee of University Hospitals Leuven (B322201838047) and informed written consent was obtained from all the participants, when applicable.

### Placental DNA Sample Collection and Processing

After delivery, placental tissue samples were taken from patients and controls for DNA extraction (Table 1). Placental DNA extraction was performed using the DNeasy Blood & Tissue Kit (QIAGEN).

TABLE 1
Clinical characteristics of placental DNA samples
from preeclamptic cases and control pregnancies

| | Case | Control | P value |
|---|---|---|---|
| Amount | 11 | 26 | |
| Caucasian / Non-Caucasian | 11 / 0 | 23 / 3 | 0.61^{#} |
| Maternal age (years, Mean ± SD, | 30.5 ± 3.9 | 29.2 ± 4.24 | 0.25^{§} |
| [range]) | [22-35] | [20-38] | |
| Gestational age at sampling | 223 ± 20 | 272 ± 10 | <0.001^{§} |
| (days, Mean ± SD [range]) | [172-246] | [247-284] | |
| Body mass index (kg/m2, | 24.3 ± 4.9 | 25.0 ± 5.4 | 0.83^{§} |
| Mean ± SD, [range]) | [17.7-33.3] | [18.6-42.9] | |
| Nulliparous / Multiparous | 8 / 3 | 13 / 13 | 0.36^{#} |
| Previous pregnancy without / with | 1 / 2 | 13 / 0 | 0.029^{#} |
| preeclampsia | | | |
| History of smoking (Yes / No / | 1 / 9 / 1 | 5 / 21 / 0 | 0.87^{#} |
| NA) | | | |
| Aspirin intake (Yes / No) | 2 / 9 | 1 / 25 | 0.42^{#} |

P values calculated according to
^{#}chi-square or
^{§}Wilcoxon signed-rank test

Bisulfite Conversion and Library Preparation 500 ng of placental DNA was treated with bisulfite using EZ DNA Methylation-Lightning Kit (Zymo Research). Library preparation was carried out within one hour after bisulfite treatment using the ACCEL-NGS® 1S PLUS DNA Library Kit (Westburg, The Netherlands) which uses an adaptase-induced tailing step of the bisulfite-converted DNA prior to library preparation. The protocol was modified and optimized in house in order to allow the procession of bisulfite converted DNA as previously described (Galle et al., Clin Epigenetics. 2020 Feb. 14; 12(1):27). Following amplification using KAPA HiFi HotStart Uracil+ ReadyMix (Roche, Belgium), DNA library concentrations were quantified using Nanodrop, and fragment lengths were analyzed using Bioanalyzer HS. Up to 16 samples, each with uniquely indexes, were pooled together in an equimolar fashion before target enrichment. Care was taken to include case and control samples for processing and analysis in the same batch, and mixed in the same capture pool, to avoid batch-induced artefacts.

### Selection of CpG Targets, Probe Design and Bisulfite-DNA Capture

We performed an enrichment of targets of interest prior to sequencing to avoid costly whole-genome bisulfite sequencing. A custom set of capture probes was designed to specifically profile 34,735 regions of interest. This customized set encompasses all 25,295 region containing one or more CpGs with a low level of methylation (average methylation level <3%) in blood as measured in healthy controls using the Illumina 450K array (Hannum et al., Molecular Cell 2013, 24 Jan.; 49(2):359), and an additional 10,011 region that show high methylation in placental tissue (methylation level >30%) but low methylation in blood (methylation level <10%) based on whole-genome bisulfite sequencing data (Court et al., Genome Res. 2014 April; 24(4): 554; Kunde-Ramamoorthy et al., Nucleic Acids Res. 2014 April; 42(6): e43). 571 regions overlap in both datasets. The design and production of the corresponding capture probes was done by Roche NimbleGen (Pleasanton, CA, USA). Target enrichment was performed as previously described (Galle et al., Clin Epigenetics. 2020 Feb. 14; 12(1):27), and the resulting libraries were sequenced in house on an Illumina HiSeq4000.

### Sequencing Data Analysis

Sequencing reads were processed with TrimGalore (Krueger, F. Trim Galore! https://web.archive.org/web/20200915171508/http://www.bioinformatics.babraha m.ac.uk/projects/trim_galore/—version 0.6.4_dev) to remove potential adapter contamination, and to trim off the adaptase-induced addition of random nucleotides. Next, trimmed reads were mapped to human genome build Grch37/hg19 using Bismark (v0.22.3), with multi-seed length of 20 bp and 1 mismatch. Duplicate copies of reads, having identical start and end sites, were subsequently removed, and the remaining, deduplicated reads were used for methylation calling using Bismark (Krueger & Andrews, Bioinformatics. 2011 Jun. 1; 27(11):1571-2). Further analysis was performed using R (R Core Team. R: A Language and Environment for Statistical Computing. R Foundation for Statistical Computing 2020)—v3.6.2). The methylation level of each region of interest was defined as the ratio of methylated C bases in a CpG context, relative to the total detected C bases (methylated and unmethylated C bases) in a CpG context in this region. Regions of interest with less than 20 quantified CpGs were considered as NA and regions which contain NA in more than 5% of samples were removed. For elastic net analyses, regions with missing values were imputed by the average methylation level for this locus across samples. Samples having estimated bisulfite conversion rates below 98.5% (as estimated by the CH methylation) or fewer than 50 CpGs quantified for DNA methylation in over 30% of regions of interest were filtered out as low-quality samples.

### Selection of Informative Loci and Elastic Net Analyses.

The R package limma (Ritchie et al., Nucleic Acids Res. 2015 Apr. 20; 43(7): e47) was used to perform a moderated t-test, contrasting DNA methylation levels between cases and controls. The Benjamini-Horchberg method was used by limma to adjust p-values for multiple testing. The 200 most significantly different regions-of-interest were taken forward for unsupervised hierarchical clustering according Ward D's method and visualized using the R package heatmap.2. Next, models were built using a random subset of 80% of all included samples. The 200 most significantly different regions-of-interest from this subset of samples were identified and used as input for building a Lasso and an elastic-net regularized generalized linear model using R package Glmnet. (Friedman & Hastie, J Stat Softw. 2010; 33(1): 1-22) The in-built 10-fold cross-validation function was used to select optimal tuning parameter λ for the model. Different elastic net mixing parameters α were tested in model building, and the optimum (α=0.2) was used throughout all analyses presented. The resultant model was applied to the remaining 20% of samples to estimate model performance in an independent dataset. 500 iterations were done to assess the associated variance. To validate these analyses, we also randomized the “case” and “control” labels and built a model 500 times.

### Results and Conclusions

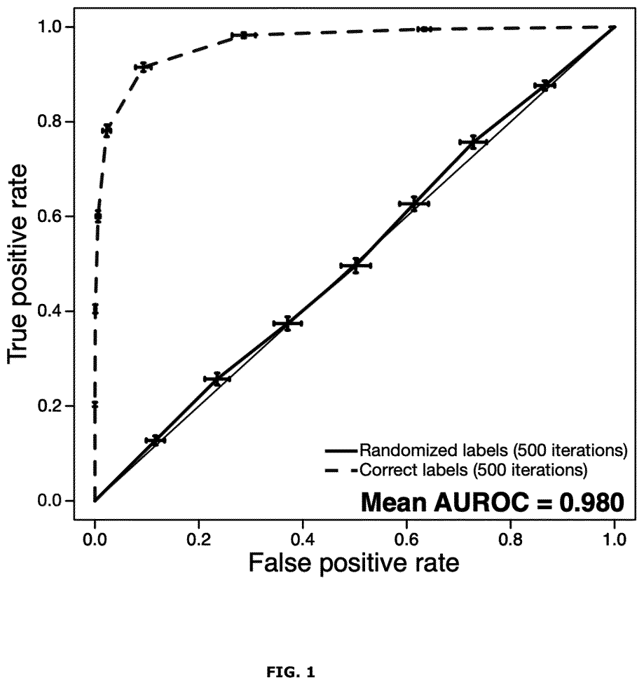

Placental DNA methylation from pregnancies complicated by preeclampsia (n=11) or control pregnancies (n=26) (Table 1) was analyzed. We applied stringent data quality filters (see Methods hereinabove), and next correlated the presence of preeclampsia with methylation levels using moderated t-statistics. Out of 33,123 informative regions, 939 and 1,560 showed significant hyper- and hypomethylation respectively (P<0.05), and 1 and 18 survived multiple-testing correction (FDR<5%). Unsupervised hierarchical clustering demonstrated that case and control samples invariably grouped separately. To assess if DNA methylation levels were sufficient to predict whether placentas were from preeclamptic pregnancies, we randomly selected a training dataset consisting of 80% of the samples from our data, and built a generalized linear model using a 10-fold cross-validation elastic net analysis. The resultant optimal model was applied on the remaining 20% of samples, which enabled us to calculate specificity and sensitivity using the Area Under the Receiver Operating Characteristics (AUROC), which reflect the diagnostic ability of a binary classifier. After 500 iterations of rebuilding and testing model, we measured an average AUROC of 0.980, which was significantly different from the AUROC of 0.50 when randomly reassigning case and control labels (FIG. 1). These data demonstrate that preeclampsia is associated with changes in DNA methylation in the placenta. Changes were predominantly found near genes with ontologies associated to processes involved in development as well as stress and immune responses, in line with the known pathophysiology of preeclampsia.

### Example 2: Analysis of cfDNA Methylation Profile at Time of Diagnosis

### Materials and Methods

Materials and Methods were identical to those of Example 1, except for the following.

cfDNA Sample Collection and Processing

Blood samples were taken for cfDNA extraction from patients at the time of preeclampsia diagnosis (between 20 and 34 weeks of gestation), or from controls at a similar moment in gestation (Table 2). Blood samples were collected into Cell-Free DNA collection tubes (Roche Diagnostics, Germany). Standard centrifugation was used for plasma isolation. CfDNA extraction was performed automatically, using the Maxwell HT cfDNA kit (Promega) on the Hamilton Liquid Handler according to the manufacturer's recommendations.

TABLE 2
Clinical characteristics of cfDNA samples at
preeclampsia diagnosis and matching controls

| | Case | Control | P value |
|---|---|---|---|
| Amount | 21 | 9 | |
| Caucasian / Non-Caucasian | 20 / 1 | 8 / 1 | 1^{#} |
| Maternal age (years, Mean ± SD, | 30.3 ± 3.9 | 30.2 ± 3.8 | 0.856^{§} |
| [range]) | [22-38] | [25-37] | |
| Gestational age at sampling | 214 ± 22 | 210 ± 18 | 0.377^{§} |
| (days, Mean ± SD [range]) | [167-241] | [171-231] | |
| Body mass index (kg/m², | 25.0 ± 5.1 | 23.6 ± 4.23 | 0.44^{§} |
| Mean ± SD, [range]) | [17.6-33.3] | [18.7-29.0] | |
| Nulliparous / Multiparous | 15 / 6 | | 0.72^{#} |
| Previous pregnancy without / with | 4 / 2 | 5 / 4 | 0.214^{#} |
| preeclampsia | | 4 / 0 | |
| History of smoking (Yes / No / | 2 / 17 / 2 | 1 / 8 /0 | 1^{#} |
| NA) | | | |
| Aspirin intake (Yes / No) | 6 / 15 | 0 / 9 | 0.20^{#} |

P values calculated according to
^{#}chi-square or
^{§}Wilcoxon signed-rank test

### Bisulfite Conversion and Library Preparation

10 to 20 μL of cfDNA was treated as described in example 1.

### Results and Conclusions

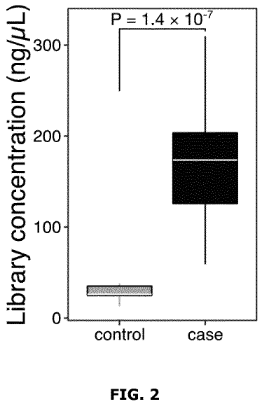

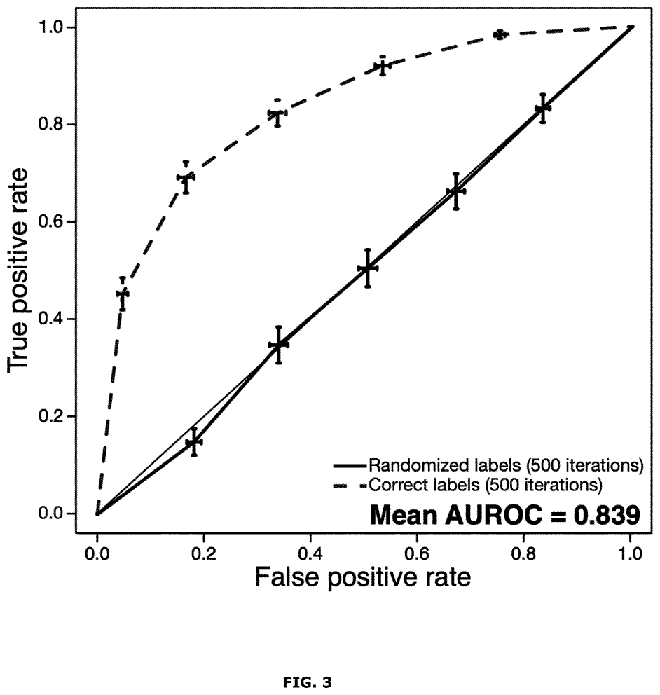

cfDNA methylation obtained from preeclampsia patients at the time of diagnosis (n=21) and matched controls (n=9) (Table 2) was analyzed to assess if DNA methylation changes were similarly evident in this minimally invasively obtained material. Notably, we observed striking differences between cfDNA concentrations from preeclampsia patients and from controls, as library yields were on average 10-fold higher (FIG. 2; P=1.4×10⁻⁷). We also observed differences in DNA methylation between cases and controls in these samples, with 466 and 2,345 of 30,203 informative regions showing significant DNA hyper- and hypomethylation (P<0.05), and hypomethylation at 1 region surviving multiple testing correction (FDR<5%). Remarkably, although there was a significant overlap between regions showing differential methylation in placental tissue and cfDNA (1.4-fold enrichment; P=0.018), a large fraction of regions also showed changes independent from those observed in placental DNA, suggesting that the cfDNA methylation changes in part reflect placenta-independent aspects of preeclampsia. Unsupervised hierarchical clustering unambiguously classified most cases and controls separately. A 10-fold cross-validation of an elastic net analysis moreover confirmed the diagnostic potential, as we measured an average AUROC of 0.839, which was significantly different from the AUROC of 0.50 when randomly assigning case and control labels (FIG. 3). These data demonstrate that preeclampsia is associated with changes in cfDNA methylation at the time of diagnosis.

### Example 3: Analysis of cfDNA Methylation Profile Before the Onset of Symptoms

### Materials and Methods

Materials and Methods were identical to those of Examples 1 and 2, except for the following.

cfDNA Sample Collection and Processing

Women diagnosed with preeclampsia and matched controls provided blood samples for non-invasive prenatal testing for common trisomies, which is routinely carried out at our center (Table 3). These cfDNA samples were taken well before the onset of early preeclampsia symptoms, at 10 to 12 weeks of gestation, and left-over cfDNA material was stored for 1 year after analysis. cfDNA was extracted from plasma from the samples as described above.

TABLE 3
Clinical characteristics of presymptomatic cfDNA samples

| | Case | Control | P value |
|---|---|---|---|
| Amount | 26 | 10 | |
| Caucasian / Non-Caucasian | 26 / 0 | 9 / 1 | 0.61^{#} |
| Maternal age (years, Mean ± SD, | 29.7 ± 4.3 | 31.2 ± 4.1 | 0.5^{§} |
| [range]) | [20-38] | [25-37] | |
| Gestational age at sampling | 85.7 ± 6.1 | 86.3 ± 5.1 | 0.90^{§} |
| (days, Mean ± SD [range]) | [67-101] | [78-94] | |
| Body mass index (kg/m², | 24.6 ± 3.8 | 23.0 ± 3.8 | 0.32^{§} |
| Mean ± SD, [range]) | [18.6-32.0] | [18.6-29.7] | |
| Nulliparous / Multiparous | 19 / 7 | 6 / 4 | 0.72^{#} |
| Previous pregnancy without / with | 3 / 4 | 4 / 0 | 0.21^{#} |
| preeclampsia | | | |
| History of smoking (Yes / No / | 2 / 22 / 2 | 5 / 21 / 0 | 1^{#} |
| NA) | | | |
| Aspirin intake (Yes / No) | 9 / 17 | 0 / 10 | 0.086^{#} |

P values calculated according to
^{#}chi-square or
^{§}Wilcoxon signed-rank test

### Results and Conclusions

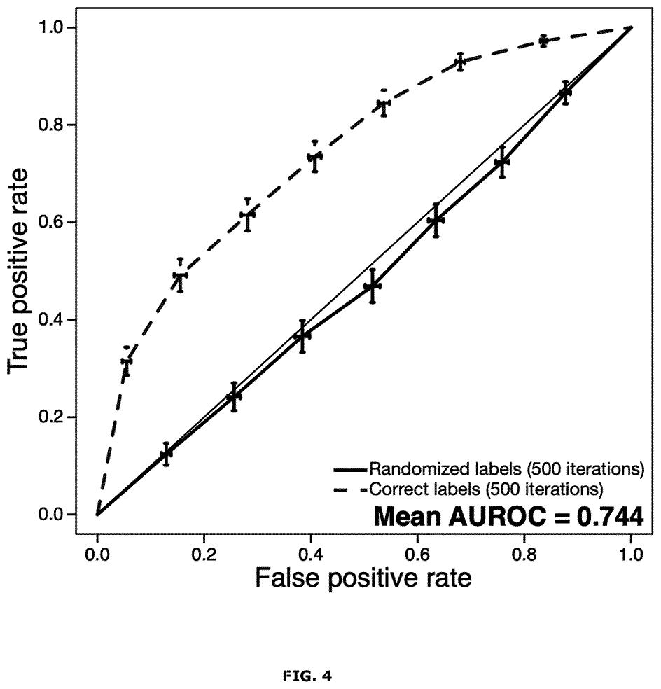

We assessed if DNA methylation changes could also be observed earlier in the pregnancy, before the clinical manifestation of preeclampsia, in a time window relevant for therapeutic intervention. The cfDNA samples were taken well before the onset of symptoms, at 10 to 12 weeks of gestation (Table 3), and left-over cfDNA material was stored for 1 year after analysis. This allowed us to quantify methylation in cfDNA samples from pregnancies that would go on to develop preeclampsia. We analyzed 26 cases and 10 controls and observed that 348 and 1,195 of 30,456 informative regions showing significant DNA hyper- and hypomethylation (P<0.05). 600 regions (SEQ ID NO: 1 to 600) showed a P value below 0.02, indicating particularly informative regions. While none of these regions alone survived multiple testing correction, unsupervised hierarchical clustering of the 200 regions with the lowest P values (SEQ ID NO: 1 to 200—P value below 0.0058) unambiguously classified all cases and controls separately, indicating that a uniform signal was detected. Crucially, to confirm the diagnostic potential at this earlier time point, we again performed a 10-fold cross-validation elastic net binominal logistic regression. Here, we measured in 500 iterations an average AUROC of 0.744, which was significantly better than the AUROC of 0.50 when randomly assigning case and control labels (FIG. 4; P<10⁻¹⁶). We also assessed whether DNA methylation changes were particularly evident in patients with a prior pregnancy complicated by preeclampsia, or in twin pregnancies, both well-established preeclampsia risk factors. Interestingly, while removing the 6 twin pregnancies yielded AUROCs similar to when removing 6 randomly selected cases (500 iterations, AUROC=0.706 versus 0.698; Welch Two Sample t-test P=0.64), we did see a significant AUROC reduction when removing the 4 cases with a prior history of preeclampsia (500 iterations, AUROC=0.631 versus 0.707 for 4 random cases removed; Welch Two Sample t-test P=6.3×10⁻⁷). While these represent only a very limited set of cases with a positive history, it suggests that part of the DNA methylation differences can be ascribed to the processes underlying the strongly increased risk associated with prior preeclampsia-complicated pregnancies. That said, a highly significant predictive signal is also detectable in patients without prior preeclampsia, or in exclusively singleton pregnancies, indicating that cfDNA methylation can predict preeclampsia in such lower-risk cohort as well. When comparing the 1543 genomic regions informative for the prediction of preeclampsia with the 2811 regions reflecting preeclampsia at the time of diagnosis, it is noteworthy to note that only 157 regions are common in the two sets. This finding highlights the fact that the predictive value of DNA methylation profile could not be anticipated from observation reflecting the association between preeclampsia and change in the DNA methylation profiles at the time of diagnosis or later during pregnancy. In conclusion, we propose that measuring cfDNA methylation can be used to stratify pregnancies for their preeclampsia risk. Importantly, the sampling window tested here corresponds to 12±1 weeks of gestation, which is prior to the 16 weeks gestational age at which prophylactic therapy needs to be initiated. It also corresponds to that of the non-invasive prenatal test, which is routinely applied to detect trisomies. This renders cfDNA methylation-based preeclampsia risk assessment achievable as a companion test to routine non-invasive prenatal test, which enhances feasibility by leveraging an accessible and logistically established sampling pipeline.

### Example 4

### Material and Methods

Women diagnosed with preeclampsia and matched controls provided blood samples for non-invasive prenatal testing for common trisomies, which is routinely carried out at our center. These cfDNA samples were taken well before the onset of early preeclampsia symptoms, at 10 to 12 weeks of gestation, and left-over cfDNA material was stored for 1 year after analysis. cfDNA was extracted from plasma from the samples as described above.

We analyzed 58 cases and 44 controls, in total 102 subjects, to assess methylation signals that may detect preeclampsia-complicated pregnancies. The elastic net model was used to perform the analysis. The elastic net algorithm estimates the model regression coefficients by minimizing the residual sum of squares and imposing a penalty on the size of regression coefficients concurrently. The penalty can cause the regression coefficients to shrink to zero, meaning that DNA methylation at those regions with a zero-coefficient is not contributing to distinguish cases and controls. Regions with non-zero coefficients therefore are regarded as signals that classify cases. Thus this model performs both selection of features (i.e. regions where DNA methylation is measured) and classification of subjects. Model fitting and parameter tuning by tenfold CV were carried out on the dataset. During each replicate of CV, models were fitted using ninefold of the data. The specified penalty parameter would shrink the coefficients of less important methylation regions to zero. A model based on methylation regions with non-zero coefficients (effective regions) was built and tested on the held-out fold to estimated classification performance. Parameters that resulted in best average performance across replicates were chosen to build a final model and regions with non-zero coefficients (N=2872-SEQ ID 601 to SEQ ID 3472) were obtained. Further region selection was performed based on magnitude of coefficient, which indicates potential importance of the region to classify cases, with a higher magnitude indicating a higher likelihood. The coefficients can be positive or negative values, indicating potential different relationships between the DNA methylation at these regions, and preeclampsia-associated signals. The absolute value of the coefficient, i.e. a non-negative value of the coefficient regardless of the sign, was used to rank the genomic regions. Table 4 includes the absolute coefficient value for each genomic region.

TABLE 4
genomic regions with non-zero coefficients. Coordinates
are indicated on the basis of Grch37/hg19.

| SEQ ID | | | | | Absolute |
|---|---|---|---|---|---|
| NO | Chromosome | Start | End | Coefficient | Coefficient |
| 601 | X | 129305897 | 129306432 | 0.44563298 | 0.445632984 |
| 602 | 5 | 14666162 | 14666315 | −0.3391018 | 0.339102 |
| 603 | 10 | 121261920 | 121262385 | 0.33633071 | 0.33633071 |
| 604 | 19 | 45579767 | 45579906 | −0.3126592 | 0.312659 |
| 605 | 5 | 172660748 | 172662340 | −0.3089095 | 0.308909 |
| 606 | 17 | 46125236 | 46125390 | 0.29877236 | 0.298772364 |
| 607 | 19 | 46997123 | 46997745 | −0.2956219 | 0.295622 |
| 608 | 9 | 123659424 | 123659604 | 0.29275749 | 0.292757493 |
| 609 | 20 | 30457964 | 30458146 | −0.2831058 | 0.283106 |
| 610 | 14 | 99728843 | 99729428 | 0.27668358 | 0.276683583 |
| 611 | 19 | 47983591 | 47983723 | 0.27541626 | 0.275416256 |
| 612 | 19 | 14063060 | 14063265 | −0.2738292 | 0.273829 |
| 613 | 15 | 39915783 | 39915960 | 0.26482185 | 0.264821848 |
| 614 | 13 | 24270029 | 24270358 | −0.2590873 | 0.259087 |
| 615 | 15 | 55700355 | 55700678 | 0.24821106 | 0.248211065 |
| 616 | 1 | 224303916 | 224303990 | −0.2460281 | 0.246028 |
| 617 | 2 | 219858170 | 219858315 | −0.2421708 | 0.242171 |
| 618 | 3 | 197237075 | 197237220 | 0.23891608 | 0.238916081 |
| 619 | 7 | 44788842 | 44788989 | −0.232955 | 0.232955 |
| 620 | 1 | 145548844 | 145549155 | 0.23283134 | 0.232831345 |
| 621 | 6 | 5085328 | 5085471 | −0.2319828 | 0.231983 |
| 622 | 11 | 10955451 | 10955605 | −0.2246658 | 0.224666 |
| 623 | 22 | 31556077 | 31556395 | −0.2226565 | 0.222656 |
| 624 | 3 | 122727816 | 122727966 | 0.22191555 | 0.221915551 |
| 625 | 1 | 201438029 | 201438172 | −0.2198085 | 0.219808 |
| 626 | 11 | 830241 | 830397 | 0.21941195 | 0.21941195 |
| 627 | 17 | 40897048 | 40897223 | −0.2190793 | 0.219079 |
| 628 | 3 | 53916270 | 53916406 | 0.21757986 | 0.21757986 |
| 629 | 16 | 31885329 | 31885538 | −0.2164828 | 0.216483 |
| 630 | 12 | 65217972 | 65219708 | −0.216439 | 0.216439 |
| 631 | 3 | 180630632 | 180630770 | 0.2135661 | 0.213566103 |
| 632 | 12 | 100536359 | 100536504 | 0.2134085 | 0.213408496 |
| 633 | 1 | 214360767 | 214360984 | −0.2111466 | 0.211147 |
| 634 | 12 | 110841850 | 110841978 | −0.2104868 | 0.210487 |
| 635 | 5 | 141348678 | 141348821 | −0.2100991 | 0.210099 |
| 636 | 1 | 181287210 | 181288196 | −0.2096319 | 0.209632 |
| 637 | 17 | 73936994 | 73937378 | −0.2088426 | 0.208843 |
| 638 | 14 | 102258363 | 102258534 | −0.2087761 | 0.208776 |
| 639 | 15 | 85874132 | 85874271 | −0.2086528 | 0.208653 |
| 640 | 18 | 56936803 | 56936927 | 0.20820551 | 0.208205507 |
| 641 | 8 | 134583343 | 134583484 | −0.2079382 | 0.207938 |
| 642 | 2 | 109237708 | 109238030 | 0.20701051 | 0.207010514 |
| 643 | 9 | 36191112 | 36191253 | 0.20579081 | 0.205790809 |
| 644 | 17 | 74380748 | 74380893 | 0.20455692 | 0.204556918 |
| 645 | 14 | 20773860 | 20774004 | −0.2040397 | 0.20404 |
| 646 | 22 | 39542051 | 39542192 | −0.203744 | 0.203744 |
| 647 | 3 | 113416565 | 113416707 | −0.2036623 | 0.203662 |
| 648 | 3 | 169898990 | 169899133 | 0.2031194 | 0.203119405 |
| 649 | 3 | 115377513 | 115377648 | 0.20236239 | 0.20236239 |
| 650 | 19 | 1207046 | 1207185 | 0.20148065 | 0.201480651 |
| 651 | 12 | 27175319 | 27175590 | −0.2006048 | 0.200605 |
| 652 | 1 | 110052274 | 110052631 | 0.20034111 | 0.200341112 |
| 653 | 19 | 18533462 | 18533598 | 0.19816916 | 0.198169161 |
| 654 | 10 | 106113532 | 106113683 | 0.19743232 | 0.197432316 |
| 655 | 1 | 16399983 | 16400137 | −0.1963359 | 0.196336 |
| 656 | 7 | 65958904 | 65959051 | −0.1959121 | 0.195912 |
| 657 | 10 | 74451767 | 74452017 | 0.19540493 | 0.195404926 |
| 658 | 4 | 77997849 | 77997993 | −0.1927314 | 0.192731 |
| 659 | 4 | 6711918 | 6712064 | −0.1917151 | 0.191715 |
| 660 | 4 | 156874695 | 156874828 | 0.19039925 | 0.19039925 |
| 661 | 7 | 32982874 | 32983041 | −0.1896368 | 0.189637 |
| 662 | 2 | 61108815 | 61108965 | −0.1896119 | 0.189612 |
| 663 | 4 | 187111964 | 187112222 | 0.18921565 | 0.189215654 |
| 664 | 11 | 48077382 | 48077816 | −0.1890818 | 0.189082 |
| 665 | 1 | 111991452 | 111991590 | −0.188931 | 0.188931 |
| 666 | 10 | 95314871 | 95315050 | −0.1887875 | 0.188788 |
| 667 | 2 | 45169309 | 45170662 | −0.1878297 | 0.18783 |
| 668 | 5 | 65017179 | 65017316 | −0.1869053 | 0.186905 |
| 669 | 1 | 25665084 | 25665218 | 0.18658822 | 0.186588219 |
| 670 | 13 | 32889784 | 32889943 | 0.18609901 | 0.186099012 |
| 671 | 20 | 31553445 | 31553591 | 0.1859039 | 0.185903898 |
| 672 | 5 | 52284829 | 52284974 | 0.18518667 | 0.18518667 |
| 673 | 17 | 76352467 | 76352752 | 0.18254277 | 0.182542768 |
| 674 | 11 | 44327719 | 44328118 | −0.1823371 | 0.182337 |
| 675 | 1 | 6526320 | 6526459 | 0.18194678 | 0.181946784 |
| 676 | 4 | 13543358 | 13544954 | −0.1813965 | 0.181396 |
| 677 | 5 | 43018204 | 43018361 | 0.18099528 | 0.18099528 |
| 678 | 17 | 80291698 | 80291837 | 0.17964418 | 0.179644175 |
| 679 | 16 | 89752933 | 89753078 | −0.1795752 | 0.179575 |
| 680 | 8 | 41649650 | 41650200 | 0.17877141 | 0.178771408 |
| 681 | 11 | 64064904 | 64065125 | −0.1784622 | 0.178462 |
| 682 | 1 | 119543994 | 119544140 | 0.1782101 | 0.178210097 |
| 683 | 19 | 34919020 | 34919234 | 0.17786967 | 0.177869669 |
| 684 | 1 | 16142234 | 16142374 | 0.17717016 | 0.177170165 |
| 685 | 6 | 26313342 | 26313484 | −0.1769268 | 0.176927 |
| 686 | 17 | 29025182 | 29025306 | 0.17666937 | 0.176669374 |
| 687 | 14 | 81687430 | 81687820 | 0.17510023 | 0.175100234 |
| 688 | 1 | 27019010 | 27019157 | 0.17451113 | 0.174511127 |
| 689 | 11 | 3078544 | 3078689 | −0.1743488 | 0.174349 |
| 690 | 17 | 75370532 | 75370668 | 0.17416869 | 0.174168695 |
| 691 | X | 137794462 | 137794616 | 0.1737521 | 0.173752105 |
| 692 | 13 | 21099799 | 21099943 | −0.1733235 | 0.173323 |
| 693 | 11 | 8615428 | 8615754 | 0.17326636 | 0.173266356 |
| 694 | 5 | 177659508 | 177659647 | −0.172955 | 0.172955 |
| 695 | 2 | 42588715 | 42588911 | −0.1729298 | 0.17293 |
| 696 | 2 | 27546010 | 27546152 | 0.17286965 | 0.172869652 |
| 697 | 8 | 144100463 | 144100598 | 0.17244144 | 0.172441437 |
| 698 | 6 | 149805874 | 149806368 | 0.17238219 | 0.172382188 |
| 699 | 17 | 62008738 | 62008941 | 0.17159763 | 0.171597632 |
| 700 | 1 | 31274718 | 31274946 | 0.17119726 | 0.171197264 |
| 701 | 6 | 123110522 | 123110817 | 0.17104883 | 0.171048831 |
| 702 | 6 | 6461502 | 6461642 | 0.17100517 | 0.171005165 |
| 703 | 12 | 4381700 | 4381973 | 0.17066449 | 0.170664487 |
| 704 | 11 | 2287307 | 2287463 | 0.17021442 | 0.17021442 |
| 705 | 12 | 121837381 | 121837543 | 0.16859453 | 0.16859453 |
| 706 | 1 | 113249576 | 113249713 | −0.168375 | 0.168375 |
| 707 | 10 | 119135222 | 119135373 | 0.16813432 | 0.168134318 |
| 708 | 5 | 87976489 | 87976638 | −0.1676762 | 0.167676 |
| 709 | 16 | 53579404 | 53579579 | 0.16766557 | 0.167665569 |
| 710 | 3 | 194304858 | 194305011 | 0.16637488 | 0.166374879 |
| 711 | 1 | 26438766 | 26438917 | 0.16636018 | 0.166360178 |
| 712 | 16 | 84788210 | 84788383 | 0.16528602 | 0.165286015 |
| 713 | 19 | 40596446 | 40596598 | 0.16473773 | 0.164737732 |
| 714 | 6 | 143265730 | 143265882 | 0.16411617 | 0.164116166 |
| 715 | 16 | 75182490 | 75182636 | −0.1630638 | 0.163064 |
| 716 | 14 | 105161759 | 105161932 | 0.16281328 | 0.162813277 |
| 717 | 4 | 149756225 | 149756362 | 0.16195863 | 0.161958629 |
| 718 | 7 | 87228695 | 87229050 | −0.1619133 | 0.161913 |
| 719 | 16 | 4896864 | 4897003 | 0.16189975 | 0.161899749 |
| 720 | 2 | 60962943 | 60963091 | 0.1616802 | 0.161680205 |
| 721 | 5 | 74162541 | 74162681 | −0.1605474 | 0.160547 |
| 722 | 1 | 150898592 | 150898844 | −0.1602376 | 0.160238 |
| 723 | 11 | 62554757 | 62554944 | −0.1601918 | 0.160192 |
| 724 | 19 | 59073822 | 59073969 | −0.1596988 | 0.159699 |
| 725 | 15 | 74753793 | 74754073 | 0.15793616 | 0.157936162 |
| 726 | 10 | 60028893 | 60029042 | −0.1574458 | 0.157446 |
| 727 | 10 | 7860431 | 7860638 | 0.15726279 | 0.157262792 |
| 728 | 12 | 54673321 | 54673466 | 0.15672621 | 0.156726207 |
| 729 | 9 | 123605161 | 123605302 | 0.15523049 | 0.155230486 |
| 730 | 1 | 59282111 | 59282245 | −0.1551621 | 0.155162 |
| 731 | 2 | 182451457 | 182451598 | −0.1548641 | 0.154864 |
| 732 | 1 | 31538800 | 31539002 | −0.1536343 | 0.153634 |
| 733 | 11 | 59328387 | 59328511 | 0.15320323 | 0.153203227 |
| 734 | 16 | 68119626 | 68119775 | 0.15307605 | 0.153076051 |
| 735 | 12 | 120032192 | 120033349 | −0.1526535 | 0.152654 |
| 736 | 15 | 71841464 | 71841603 | 0.15254958 | 0.152549579 |
| 737 | 9 | 4490236 | 4490385 | 0.15240673 | 0.15240673 |
| 738 | 17 | 4642826 | 4643009 | 0.15203742 | 0.152037423 |
| 739 | 16 | 9184406 | 9184556 | −0.1514956 | 0.151496 |
| 740 | 11 | 62446518 | 62446803 | 0.15104683 | 0.151046834 |
| 741 | 19 | 37064079 | 37064446 | 0.15094702 | 0.150947022 |
| 742 | 17 | 49230802 | 49231010 | −0.1508865 | 0.150887 |
| 743 | 11 | 71814143 | 71814284 | −0.1502715 | 0.150272 |
| 744 | 2 | 26569043 | 26569180 | 0.15015322 | 0.150153222 |
| 745 | 16 | 58163124 | 58163437 | 0.14947825 | 0.14947825 |
| 746 | 6 | 100036716 | 100036860 | 0.14945169 | 0.149451692 |
| 747 | 4 | 120548520 | 120548689 | 0.14886801 | 0.148868013 |
| 748 | 4 | 2395700 | 2395842 | 0.14876985 | 0.148769852 |
| 749 | 5 | 16466251 | 16466390 | 0.1487424 | 0.148742395 |
| 750 | 17 | 79269279 | 79269553 | 0.14712416 | 0.147124156 |
| 751 | 2 | 237072323 | 237072954 | −0.1466052 | 0.146605 |
| 752 | 7 | 38346718 | 38346878 | −0.1465019 | 0.146502 |
| 753 | 19 | 44809119 | 44809294 | −0.1463524 | 0.146352 |
| 754 | 8 | 82754015 | 82754165 | 0.14630639 | 0.146306387 |
| 755 | 2 | 73143215 | 73143394 | 0.14619245 | 0.146192454 |
| 756 | 1 | 91184429 | 91185043 | −0.1461653 | 0.146165 |
| 757 | 16 | 85588585 | 85588724 | 0.14612756 | 0.146127562 |
| 758 | 18 | 42339153 | 42339337 | 0.14516184 | 0.14516184 |
| 759 | 16 | 53737996 | 53738251 | 0.14490709 | 0.144907086 |
| 760 | 8 | 143750782 | 143750918 | 0.14468827 | 0.144688271 |
| 761 | 12 | 69863934 | 69864144 | −0.1445492 | 0.144549 |
| 762 | 17 | 4607145 | 4607287 | 0.14432771 | 0.144327707 |
| 763 | 6 | 32805926 | 32806073 | −0.1441882 | 0.144188 |
| 764 | 7 | 87505057 | 87505259 | 0.14413904 | 0.144139045 |
| 765 | 12 | 122277445 | 122277586 | −0.1440071 | 0.144007 |
| 766 | 3 | 15469306 | 15469457 | 0.14369601 | 0.143696006 |
| 767 | 1 | 91188844 | 91189889 | −0.1436102 | 0.14361 |
| 768 | 1 | 151584579 | 151584830 | −0.1434694 | 0.143469 |
| 769 | 2 | 241560947 | 241561325 | −0.143447 | 0.143447 |
| 770 | 5 | 87955336 | 87955529 | 0.14327831 | 0.143278313 |
| 771 | 7 | 158622788 | 158622930 | 0.14310712 | 0.143107116 |
| 772 | 20 | 42875703 | 42875826 | 0.14309651 | 0.143096507 |
| 773 | 6 | 146136362 | 146136500 | −0.142609 | 0.142609 |
| 774 | 20 | 44519861 | 44520264 | 0.14179525 | 0.141795255 |
| 775 | 20 | 4666672 | 4666832 | −0.1409467 | 0.140947 |
| 776 | 14 | 70721563 | 70721663 | −0.1404319 | 0.140432 |
| 777 | 1 | 192777808 | 192778243 | 0.13975708 | 0.13975708 |
| 778 | 17 | 4981523 | 4981694 | 0.13947292 | 0.139472916 |
| 779 | 19 | 12896948 | 12897219 | 0.13939583 | 0.13939583 |
| 780 | 16 | 58060500 | 58060652 | −0.1391027 | 0.139103 |
| 781 | 6 | 10423476 | 10423737 | −0.1389183 | 0.138918 |
| 782 | 15 | 45427318 | 45427903 | −0.1389019 | 0.138902 |
| 783 | 6 | 24774908 | 24775041 | 0.13855897 | 0.138558971 |
| 784 | 2 | 242575780 | 242575919 | −0.13848 | 0.13848 |
| 785 | 10 | 72164058 | 72164204 | −0.1379511 | 0.137951 |
| 786 | 4 | 184328243 | 184328528 | 0.13790585 | 0.13790585 |
| 787 | 6 | 37515404 | 37515553 | −0.1378394 | 0.137839 |
| 788 | 7 | 98477652 | 98477790 | −0.1378335 | 0.137834 |
| 789 | 2 | 128848638 | 128849039 | 0.13771356 | 0.137713562 |
| 790 | 2 | 160918606 | 160918702 | 0.13770678 | 0.13770678 |
| 791 | 2 | 73518582 | 73519190 | −0.1369057 | 0.136906 |
| 792 | 6 | 42013271 | 42013411 | 0.13648958 | 0.136489576 |
| 793 | 2 | 95831341 | 95831484 | −0.13629 | 0.13629 |
| 794 | 19 | 50370422 | 50370557 | −0.1360876 | 0.136088 |
| 795 | 2 | 12628634 | 12628768 | 0.13581945 | 0.135819449 |
| 796 | 19 | 9938784 | 9938932 | 0.13562818 | 0.135628184 |
| 797 | 11 | 65101083 | 65101252 | 0.13556468 | 0.135564681 |
| 798 | 12 | 32552448 | 32552599 | 0.13545152 | 0.135451524 |
| 799 | 5 | 176449540 | 176449712 | 0.13464975 | 0.134649749 |
| 800 | 2 | 219575919 | 219576066 | 0.13444574 | 0.134445743 |
| 801 | 4 | 6576506 | 6576641 | −0.1340184 | 0.134018 |
| 802 | 19 | 58095392 | 58095539 | 0.13327157 | 0.133271567 |
| 803 | 20 | 54967297 | 54967580 | −0.133218 | 0.133218 |
| 804 | 17 | 78518799 | 78518946 | 0.13238008 | 0.132380079 |
| 805 | 15 | 91565536 | 91565682 | −0.1321109 | 0.132111 |
| 806 | 4 | 56915249 | 56915430 | 0.1320345 | 0.132034499 |
| 807 | 16 | 73126297 | 73126437 | 0.13165564 | 0.131655645 |
| 808 | 3 | 188850124 | 188850283 | 0.13070056 | 0.130700561 |
| 809 | 16 | 67203657 | 67204055 | −0.1306677 | 0.130668 |
| 810 | 14 | 70234664 | 70234811 | −0.1302922 | 0.130292 |
| 811 | 16 | 86774488 | 86774640 | 0.13007555 | 0.13007555 |
| 812 | 2 | 64681656 | 64681817 | −0.130035 | 0.130035 |
| 813 | 2 | 101618490 | 101618680 | 0.13000554 | 0.130005539 |
| 814 | 2 | 177016387 | 177016779 | −0.1298799 | 0.12988 |
| 815 | 14 | 21945226 | 21945492 | 0.12982989 | 0.129829885 |
| 816 | 10 | 51827577 | 51827821 | 0.12974118 | 0.129741178 |
| 817 | 3 | 169482642 | 169482783 | 0.12882668 | 0.128826678 |
| 818 | 11 | 3078187 | 3078333 | −0.1286171 | 0.128617 |
| 819 | 1 | 76540039 | 76540251 | 0.12846959 | 0.128469591 |
| 820 | 11 | 75236534 | 75236682 | 0.12823489 | 0.128234891 |
| 821 | 11 | 32851454 | 32851596 | −0.1282283 | 0.128228 |
| 822 | 2 | 219857713 | 219857996 | −0.1278666 | 0.127867 |
| 823 | 9 | 98789651 | 98790177 | −0.1278582 | 0.127858 |
| 824 | 4 | 83206036 | 83206185 | −0.1274593 | 0.127459 |
| 825 | 18 | 5237750 | 5238004 | 0.12736674 | 0.127366735 |
| 826 | 1 | 63782900 | 63783244 | 0.12686329 | 0.126863289 |
| 827 | 8 | 125740038 | 125740190 | 0.12673773 | 0.126737729 |
| 828 | 17 | 39968758 | 39968923 | 0.12657848 | 0.126578481 |
| 829 | 1 | 101644777 | 101645032 | 0.12616317 | 0.126163171 |
| 830 | 5 | 71616384 | 71616590 | 0.12573497 | 0.125734967 |
| 831 | 6 | 50674575 | 50674711 | −0.125283 | 0.125283 |
| 832 | 11 | 71950848 | 71950992 | −0.1251196 | 0.12512 |
| 833 | 10 | 82422790 | 82422933 | −0.1247334 | 0.124733 |
| 834 | 11 | 63258646 | 63258849 | −0.1247233 | 0.124723 |
| 835 | 2 | 214016862 | 214017019 | −0.1246401 | 0.12464 |
| 836 | 17 | 7155819 | 7155976 | −0.124618 | 0.124618 |
| 837 | 5 | 139283003 | 139283434 | −0.1245982 | 0.124598 |
| 838 | 3 | 13526705 | 13526888 | −0.1243574 | 0.124357 |
| 839 | 6 | 33280439 | 33280590 | −0.1240049 | 0.124005 |
| 840 | 12 | 89745594 | 89745748 | 0.12393337 | 0.123933369 |
| 841 | 1 | 100503688 | 100503829 | 0.12368455 | 0.123684553 |
| 842 | 7 | 102105562 | 102105719 | −0.1236018 | 0.123602 |
| 843 | 6 | 111197755 | 111197920 | 0.12353636 | 0.123536358 |
| 844 | 21 | 45136852 | 45136994 | 0.12338136 | 0.123381362 |
| 845 | 12 | 21590384 | 21590626 | 0.12333802 | 0.12333802 |
| 846 | 22 | 19748306 | 19748478 | 0.12328302 | 0.123283018 |
| 847 | 11 | 9336283 | 9336422 | −0.123256 | 0.123256 |
| 848 | 7 | 139025936 | 139026059 | −0.1226469 | 0.122647 |
| 849 | 8 | 143556669 | 143556806 | 0.12244968 | 0.122449678 |
| 850 | 6 | 85483884 | 85484642 | −0.1224481 | 0.122448 |
| 851 | 12 | 7052954 | 7053099 | −0.1223402 | 0.12234 |
| 852 | 7 | 76751663 | 76751919 | −0.1221124 | 0.122112 |
| 853 | 1 | 161369572 | 161369715 | 0.1219004 | 0.121900396 |
| 854 | 3 | 121553857 | 121554107 | 0.1214881 | 0.121488104 |
| 855 | 9 | 33446395 | 33446544 | −0.1214289 | 0.121429 |
| 856 | 5 | 176944005 | 176944215 | 0.12141007 | 0.121410074 |
| 857 | 16 | 10408906 | 10409054 | −0.1213667 | 0.121367 |
| 858 | X | 153267714 | 153268238 | 0.12136481 | 0.121364813 |
| 859 | 19 | 12777638 | 12777851 | 0.12134619 | 0.121346187 |
| 860 | 19 | 44668938 | 44669098 | −0.1212488 | 0.121249 |
| 861 | 8 | 37963228 | 37963273 | −0.1211178 | 0.121118 |
| 862 | 1 | 222884832 | 222884982 | 0.12091788 | 0.120917881 |
| 863 | 10 | 133741792 | 133742221 | −0.1208671 | 0.120867 |
| 864 | 12 | 25204260 | 25204403 | −0.1207576 | 0.120758 |
| 865 | 6 | 26027085 | 26027253 | 0.12062112 | 0.120621125 |
| 866 | 22 | 51222453 | 51222578 | 0.12049232 | 0.120492325 |
| 867 | 17 | 41322335 | 41322548 | 0.12017179 | 0.120171795 |
| 868 | 1 | 46806279 | 46806458 | 0.120127 | 0.120127001 |
| 869 | 3 | 142442839 | 142443020 | 0.1198196 | 0.119819599 |
| 870 | X | 68114474 | 68114653 | 0.11981341 | 0.119813414 |
| 871 | 1 | 78148299 | 78148596 | −0.1197711 | 0.119771 |
| 872 | 2 | 161263785 | 161263916 | 0.11960592 | 0.119605918 |
| 873 | 4 | 71600765 | 71600909 | −0.1193453 | 0.119345 |
| 874 | 16 | 451993 | 452130 | −0.1192503 | 0.11925 |
| 875 | 1 | 40204582 | 40204739 | 0.11915537 | 0.119155369 |
| 876 | 7 | 116962669 | 116964905 | −0.1189969 | 0.118997 |
| 877 | 6 | 143234720 | 143234854 | 0.11874702 | 0.118747019 |
| 878 | 10 | 99080324 | 99080913 | −0.1184319 | 0.118432 |
| 879 | 10 | 79793574 | 79793729 | 0.11835143 | 0.118351427 |
| 880 | 7 | 5553557 | 5553693 | 0.11820588 | 0.118205885 |
| 881 | 9 | 84270958 | 84271103 | −0.1179849 | 0.117985 |
| 882 | 8 | 100905357 | 100905487 | 0.11777037 | 0.117770375 |
| 883 | 19 | 41854800 | 41854983 | 0.11771923 | 0.117719229 |
| 884 | 6 | 30029414 | 30029587 | 0.11768694 | 0.117686938 |
| 885 | 3 | 57541673 | 57541890 | 0.11752622 | 0.117526216 |
| 886 | 20 | 48532138 | 48532394 | −0.1174388 | 0.117439 |
| 887 | 16 | 451428 | 451708 | −0.1174046 | 0.117405 |
| 888 | 15 | 75230517 | 75230701 | −0.1173994 | 0.117399 |
| 889 | 1 | 161102480 | 161102626 | −0.1171478 | 0.117148 |
| 890 | 12 | 67662645 | 67662823 | −0.1170974 | 0.117097 |
| 891 | 7 | 37960824 | 37960986 | 0.11682447 | 0.116824474 |
| 892 | X | 20339054 | 20339240 | 0.11673296 | 0.116732964 |
| 893 | 16 | 1660498 | 1660645 | −0.1164754 | 0.116475 |
| 894 | 12 | 109250908 | 109251054 | −0.1162987 | 0.116299 |
| 895 | 14 | 24605487 | 24605591 | 0.11591612 | 0.115916117 |
| 896 | 1 | 17512818 | 17513008 | 0.11581692 | 0.115816921 |
| 897 | 3 | 185655718 | 185656035 | −0.115799 | 0.115799 |
| 898 | 20 | 44519316 | 44519476 | −0.1157608 | 0.115761 |
| 899 | 14 | 22887607 | 22887755 | −0.1157445 | 0.115744 |
| 900 | 1 | 236304934 | 236305072 | −0.1156777 | 0.115678 |
| 901 | 4 | 15767705 | 15768031 | −0.1156498 | 0.11565 |
| 902 | 11 | 57227277 | 57227425 | 0.11530581 | 0.115305807 |
| 903 | 2 | 85766036 | 85766490 | 0.11509756 | 0.115097559 |
| 904 | 2 | 178483332 | 178483482 | −0.1149567 | 0.114957 |
| 905 | 5 | 134366328 | 134367457 | −0.1147255 | 0.114725 |
| 906 | 5 | 1930850 | 1930994 | −0.1146585 | 0.114658 |
| 907 | 17 | 64961370 | 64961612 | 0.11459996 | 0.114599963 |
| 908 | 3 | 156806674 | 156806809 | 0.11450139 | 0.114501391 |
| 909 | 3 | 155524116 | 155524263 | 0.11432856 | 0.114328559 |
| 910 | 7 | 108097108 | 108097251 | −0.1142278 | 0.114228 |
| 911 | 11 | 64084959 | 64085217 | 0.11405017 | 0.114050173 |
| 912 | 2 | 118979663 | 118979807 | −0.1139456 | 0.113946 |
| 913 | 11 | 101980955 | 101981204 | 0.11391098 | 0.113910982 |
| 914 | 13 | 107220517 | 107220657 | −0.1137524 | 0.113752 |
| 915 | 7 | 852958 | 853104 | 0.11349834 | 0.113498336 |
| 916 | 2 | 64938486 | 64938679 | −0.1134705 | 0.113471 |
| 917 | 16 | 3661412 | 3661546 | 0.11340323 | 0.113403227 |
| 918 | 1 | 151763787 | 151763946 | 0.11337329 | 0.113373288 |
| 919 | 19 | 7197170 | 7197414 | 0.11326876 | 0.11326876 |
| 920 | 2 | 55646906 | 55647063 | 0.11315296 | 0.113152958 |
| 921 | 9 | 80911981 | 80912122 | −0.112287 | 0.112287 |
| 922 | 22 | 25817189 | 25817337 | 0.11211234 | 0.112112344 |
| 923 | 12 | 121905459 | 121905602 | −0.1120563 | 0.112056 |
| 924 | 14 | 42074796 | 42075440 | −0.1120107 | 0.112011 |
| 925 | 6 | 40555653 | 40555792 | −0.1119887 | 0.111989 |
| 926 | 5 | 180650883 | 180651124 | −0.1118581 | 0.111858 |
| 927 | 12 | 51441983 | 51442155 | 0.11176571 | 0.111765708 |
| 928 | 9 | 70971499 | 70972367 | −0.1117505 | 0.11175 |
| 929 | 3 | 197476483 | 197476659 | 0.11136297 | 0.111362972 |
| 930 | 16 | 57126657 | 57126798 | −0.111072 | 0.111072 |
| 931 | 6 | 50805702 | 50806108 | −0.1110597 | 0.11106 |
| 932 | 11 | 95524257 | 95524398 | −0.1108722 | 0.110872 |
| 933 | 5 | 177632188 | 177632328 | −0.1105156 | 0.110516 |
| 934 | 20 | 17950869 | 17951008 | 0.11051233 | 0.110512325 |
| 935 | 3 | 33759748 | 33759897 | 0.11039502 | 0.110395017 |
| 936 | 8 | 55770758 | 55770887 | 0.10992529 | 0.109925294 |
| 937 | 22 | 17850360 | 17850515 | 0.10968743 | 0.109687435 |
| 938 | 17 | 28256514 | 28256646 | −0.1095637 | 0.109564 |
| 939 | 15 | 59063100 | 59063452 | 0.10947346 | 0.109473465 |
| 940 | 6 | 27637121 | 27637625 | −0.1094128 | 0.109413 |
| 941 | 7 | 149470205 | 149470403 | 0.10935313 | 0.109353129 |
| 942 | 11 | 77790772 | 77790918 | 0.10897854 | 0.108978536 |
| 943 | 1 | 180206299 | 180206495 | 0.10855797 | 0.108557974 |
| 944 | 1 | 181452682 | 181452959 | −0.1085418 | 0.108542 |
| 945 | 7 | 2727685 | 2727851 | −0.1080897 | 0.10809 |
| 946 | 13 | 31192009 | 31192148 | 0.10804634 | 0.108046341 |
| 947 | 17 | 67410659 | 67410869 | 0.1079289 | 0.107928896 |
| 948 | 5 | 40835114 | 40835376 | 0.10790229 | 0.107902292 |
| 949 | 7 | 149412213 | 149412361 | 0.1079018 | 0.107901799 |
| 950 | 5 | 2762209 | 2762356 | 0.10783115 | 0.107831155 |
| 951 | 19 | 10342107 | 10342305 | −0.1078061 | 0.107806 |
| 952 | 2 | 95787825 | 95788018 | −0.1077596 | 0.10776 |
| 953 | 1 | 93544930 | 93545068 | 0.10775551 | 0.107755507 |
| 954 | 1 | 239549548 | 239549682 | 0.10730266 | 0.107302664 |
| 955 | 3 | 52804829 | 52804981 | −0.1072624 | 0.107262 |
| 956 | 1 | 230203924 | 230204064 | 0.10724358 | 0.107243577 |
| 957 | 17 | 18087179 | 18087323 | −0.1072414 | 0.107241 |
| 958 | 7 | 27135043 | 27135318 | −0.1070958 | 0.107096 |
| 959 | 21 | 35014116 | 35014429 | −0.1070592 | 0.107059 |
| 960 | 8 | 21988068 | 21988227 | −0.1069173 | 0.106917 |
| 961 | 1 | 26249270 | 26249453 | 0.10669795 | 0.106697951 |
| 962 | 4 | 154388305 | 154388448 | −0.1066182 | 0.106618 |
| 963 | 8 | 122654157 | 122654305 | 0.10658841 | 0.106588406 |
| 964 | 6 | 41168492 | 41168819 | 0.10656172 | 0.106561723 |
| 965 | 18 | 74204060 | 74204205 | 0.10652318 | 0.106523178 |
| 966 | 6 | 108487080 | 108487227 | 0.10648506 | 0.106485064 |
| 967 | 15 | 97436519 | 97436677 | −0.1064841 | 0.106484 |
| 968 | 15 | 79102928 | 79103080 | 0.10645644 | 0.10645644 |
| 969 | X | 153946154 | 153946323 | 0.10643479 | 0.106434792 |
| 970 | 1 | 231229107 | 231229256 | 0.10619396 | 0.106193958 |
| 971 | 1 | 6520581 | 6520907 | 0.10619296 | 0.106192963 |
| 972 | 22 | 32870973 | 32871111 | −0.1061425 | 0.106143 |
| 973 | 10 | 70091663 | 70091940 | 0.10589077 | 0.105890771 |
| 974 | 12 | 56325659 | 56325934 | −0.1056662 | 0.105666 |
| 975 | 6 | 56708952 | 56709102 | 0.10560383 | 0.105603832 |
| 976 | 16 | 30904782 | 30905068 | −0.1053534 | 0.105353 |
| 977 | 11 | 110584009 | 110584151 | −0.1052921 | 0.105292 |
| 978 | 5 | 143549944 | 143550202 | 0.1051967 | 0.105196703 |
| 979 | 17 | 55334943 | 55335129 | 0.10504211 | 0.105042111 |
| 980 | 9 | 117350156 | 117350294 | −0.1048804 | 0.10488 |
| 981 | 17 | 18684358 | 18684681 | −0.1048608 | 0.104861 |
| 982 | 14 | 51410609 | 51410753 | −0.104763 | 0.104763 |
| 983 | 1 | 41444792 | 41444927 | 0.10468079 | 0.104680795 |
| 984 | 3 | 44803015 | 44803415 | 0.10463122 | 0.104631223 |
| 985 | 15 | 40213198 | 40213413 | −0.1043272 | 0.104327 |
| 986 | 6 | 30457994 | 30458075 | −0.1042948 | 0.104295 |
| 987 | 18 | 56939043 | 56940431 | −0.1040079 | 0.104008 |
| 988 | 8 | 141520793 | 141520975 | −0.1039451 | 0.103945 |
| 989 | 4 | 141072572 | 141072713 | 0.10391466 | 0.103914662 |
| 990 | 17 | 47785514 | 47785727 | −0.1039097 | 0.10391 |
| 991 | 10 | 72200348 | 72200698 | 0.10382647 | 0.103826468 |
| 992 | 16 | 53538124 | 53538330 | −0.1037344 | 0.103734 |
| 993 | 13 | 112728135 | 112728267 | −0.103697 | 0.103697 |
| 994 | 17 | 1466224 | 1466357 | 0.10368673 | 0.10368673 |
| 995 | 13 | 20356027 | 20356297 | 0.10366154 | 0.103661537 |
| 996 | 2 | 198171461 | 198171671 | −0.1035648 | 0.103565 |
| 997 | 11 | 130318636 | 130318788 | 0.10330692 | 0.103306917 |
| 998 | 7 | 130641546 | 130641758 | −0.1032724 | 0.103272 |
| 999 | 16 | 74402267 | 74402397 | 0.10319866 | 0.103198659 |
| 1000 | 19 | 52207515 | 52207664 | 0.10317564 | 0.103175643 |
| 1001 | 6 | 139601021 | 139601152 | 0.10316087 | 0.103160868 |
| 1002 | 18 | 54318585 | 54318734 | 0.10315762 | 0.103157621 |
| 1003 | 15 | 50646547 | 50646686 | 0.10284302 | 0.102843016 |
| 1004 | 1 | 171454757 | 171454898 | 0.10269782 | 0.102697823 |
| 1005 | 1 | 202776220 | 202776375 | 0.10259548 | 0.102595476 |
| 1006 | 19 | 41168937 | 41169082 | 0.10235072 | 0.10235072 |
| 1007 | 18 | 43266937 | 43267112 | −0.1023217 | 0.102322 |
| 1008 | 19 | 9695118 | 9695495 | −0.1021837 | 0.102184 |
| 1009 | 12 | 6961354 | 6961501 | 0.1021795 | 0.1021795 |
| 1010 | 12 | 57630785 | 57630922 | 0.10198885 | 0.101988855 |
| 1011 | 1 | 38229995 | 38230146 | −0.1018923 | 0.101892 |
| 1012 | 5 | 95997645 | 95997793 | 0.10178791 | 0.101787912 |
| 1013 | 3 | 9932664 | 9932811 | −0.1017368 | 0.101737 |
| 1014 | 8 | 101964064 | 101964220 | −0.1015481 | 0.101548 |
| 1015 | 4 | 186125264 | 186125400 | −0.1014876 | 0.101488 |
| 1016 | 2 | 198363789 | 198363943 | 0.10137944 | 0.101379439 |
| 1017 | 11 | 75081228 | 75081328 | 0.10134292 | 0.101342915 |
| 1018 | 11 | 65430458 | 65430608 | −0.1013208 | 0.101321 |
| 1019 | 15 | 76629033 | 76629178 | −0.1012398 | 0.10124 |
| 1020 | 6 | 16761862 | 16761976 | −0.1012321 | 0.101232 |
| 1021 | 1 | 152009173 | 152009323 | 0.10119709 | 0.101197093 |
| 1022 | 19 | 42760125 | 42760264 | −0.1007193 | 0.100719 |
| 1023 | 4 | 140477990 | 140478138 | 0.10042029 | 0.10042029 |
| 1024 | 5 | 133512489 | 133512637 | 0.10035109 | 0.100351086 |
| 1025 | 2 | 119603135 | 119604030 | −0.1002669 | 0.100267 |
| 1026 | 1 | 160001599 | 160001743 | 0.10013455 | 0.100134552 |
| 1027 | 17 | 27276352 | 27276483 | 0.09995143 | 0.099951427 |
| 1028 | 4 | 3076181 | 3076501 | −0.0997694 | 0.0997694 |
| 1029 | 10 | 71906506 | 71906645 | 0.09966724 | 0.099667244 |
| 1030 | 20 | 23331190 | 23331425 | 0.09942722 | 0.099427225 |
| 1031 | 7 | 128694318 | 128694458 | 0.0992848 | 0.099284801 |
| 1032 | 6 | 26457933 | 26458095 | −0.0992047 | 0.0992047 |
| 1033 | 2 | 10521826 | 10521973 | −0.0992001 | 0.0992001 |
| 1034 | 15 | 23034624 | 23034732 | 0.09885296 | 0.098852964 |
| 1035 | 12 | 511285 | 511431 | −0.09867 | 0.09867 |
| 1036 | 2 | 175351569 | 175351711 | −0.0986695 | 0.0986695 |
| 1037 | 1 | 197871760 | 197871898 | 0.09846877 | 0.098468773 |
| 1038 | 2 | 43453172 | 43453313 | −0.0983586 | 0.0983586 |
| 1039 | 4 | 175133073 | 175133211 | 0.09832648 | 0.098326482 |
| 1040 | 13 | 25875576 | 25875712 | −0.0982662 | 0.0982662 |
| 1041 | 1 | 202113996 | 202114142 | −0.098232 | 0.098232 |
| 1042 | 1 | 161129109 | 161129305 | −0.0982291 | 0.0982291 |
| 1043 | 6 | 26032330 | 26032475 | −0.0981582 | 0.0981582 |
| 1044 | 5 | 98109273 | 98109546 | 0.09792185 | 0.097921851 |
| 1045 | 8 | 144766764 | 144766910 | −0.097865 | 0.097865 |
| 1046 | 2 | 85108922 | 85109077 | 0.09745219 | 0.097452191 |
| 1047 | 2 | 64879836 | 64879977 | −0.0972865 | 0.0972865 |
| 1048 | 1 | 92350968 | 92351112 | 0.0972541 | 0.097254097 |
| 1049 | 1 | 226616927 | 226617069 | 0.09720921 | 0.097209207 |
| 1050 | 2 | 203130824 | 203130958 | 0.09715281 | 0.097152807 |
| 1051 | 9 | 139237325 | 139237448 | −0.0970797 | 0.0970797 |
| 1052 | 8 | 23315218 | 23315512 | 0.09704102 | 0.097041022 |
| 1053 | X | 80457926 | 80458088 | 0.09695349 | 0.096953492 |
| 1054 | 6 | 17280936 | 17281080 | −0.09681 | 0.09681 |
| 1055 | 15 | 80263299 | 80263450 | −0.0968037 | 0.0968037 |
| 1056 | 4 | 100738611 | 100738763 | 0.09674485 | 0.096744854 |
| 1057 | 2 | 27273349 | 27273490 | 0.09661245 | 0.096612453 |
| 1058 | 2 | 55496315 | 55496623 | 0.09641874 | 0.096418739 |
| 1059 | 7 | 140624825 | 140624975 | 0.09634679 | 0.096346794 |
| 1060 | 17 | 59940817 | 59940973 | 0.09591605 | 0.095916054 |
| 1061 | 4 | 146019259 | 146019452 | −0.095827 | 0.095827 |
| 1062 | 9 | 126101324 | 126101536 | 0.09567192 | 0.095671924 |
| 1063 | 4 | 183838657 | 183838840 | 0.09562944 | 0.095629437 |
| 1064 | 5 | 172669683 | 172670005 | −0.0954449 | 0.0954449 |
| 1065 | 16 | 73097234 | 73097648 | −0.0954079 | 0.0954079 |
| 1066 | 4 | 149363835 | 149363983 | −0.0953724 | 0.0953724 |
| 1067 | 12 | 8850653 | 8850775 | 0.09530967 | 0.095309668 |
| 1068 | 1 | 84764533 | 84764783 | 0.09508619 | 0.095086189 |
| 1069 | 2 | 176032435 | 176032658 | 0.09503209 | 0.095032089 |
| 1070 | 14 | 74226484 | 74226631 | 0.09486247 | 0.094862469 |
| 1071 | 4 | 85414011 | 85414381 | 0.09486113 | 0.094861127 |
| 1072 | 9 | 86152447 | 86153869 | −0.094802 | 0.094802 |
| 1073 | 12 | 121018942 | 121019093 | 0.09473782 | 0.094737823 |
| 1074 | 12 | 58013400 | 58013718 | −0.0945687 | 0.0945687 |
| 1075 | 17 | 80256254 | 80256595 | −0.0944905 | 0.0944905 |
| 1076 | 2 | 175458489 | 175458646 | 0.09433695 | 0.094336951 |
| 1077 | 11 | 66405841 | 66406045 | −0.0942587 | 0.0942587 |
| 1078 | 17 | 61851923 | 61852077 | −0.094239 | 0.094239 |
| 1079 | 1 | 234508974 | 234509543 | 0.09397546 | 0.09397546 |
| 1080 | 3 | 196359592 | 196359737 | 0.09392093 | 0.093920931 |
| 1081 | 1 | 156406440 | 156406799 | 0.09389821 | 0.093898214 |
| 1082 | 16 | 11759198 | 11759395 | 0.09389122 | 0.093891221 |
| 1083 | 4 | 27086368 | 27086638 | 0.09384664 | 0.093846637 |
| 1084 | 10 | 64564399 | 64564480 | −0.0938353 | 0.0938353 |
| 1085 | 6 | 82462175 | 82462312 | 0.09378751 | 0.093787512 |
| 1086 | 2 | 109210727 | 109211004 | 0.09363913 | 0.093639131 |
| 1087 | 5 | 130588789 | 130588966 | 0.09356869 | 0.093568686 |
| 1088 | 17 | 45056252 | 45056398 | −0.0933912 | 0.0933912 |
| 1089 | 22 | 38379673 | 38379816 | 0.09336527 | 0.093365275 |
| 1090 | 3 | 134093486 | 134093610 | 0.09327979 | 0.093279791 |
| 1091 | 13 | 97762213 | 97762417 | −0.0930762 | 0.0930762 |
| 1092 | 12 | 27175768 | 27175901 | 0.0930646 | 0.093064596 |
| 1093 | 17 | 46124611 | 46124791 | −0.0930593 | 0.0930593 |
| 1094 | 11 | 8933293 | 8933440 | 0.09305281 | 0.093052814 |
| 1095 | 12 | 49246043 | 49246181 | 0.09300479 | 0.093004791 |
| 1096 | 2 | 120124204 | 120124361 | 0.0929517 | 0.092951703 |
| 1097 | 2 | 26568388 | 26568535 | −0.0927396 | 0.0927396 |
| 1098 | 19 | 41035106 | 41035222 | −0.0926829 | 0.0926829 |
| 1099 | 11 | 31846277 | 31846608 | 0.09233816 | 0.092338161 |
| 1100 | 14 | 77648120 | 77648306 | 0.09228743 | 0.092287434 |
| 1101 | 6 | 132722237 | 132722384 | 0.09219349 | 0.092193491 |
| 1102 | 11 | 65029303 | 65029551 | 0.09211597 | 0.092115973 |
| 1103 | 2 | 73441631 | 73441762 | −0.0920575 | 0.0920575 |
| 1104 | 19 | 38397559 | 38397955 | 0.09203212 | 0.092032117 |
| 1105 | 19 | 15342680 | 15342815 | 0.09200319 | 0.092003192 |
| 1106 | 4 | 57843933 | 57844079 | −0.0918337 | 0.0918337 |
| 1107 | 8 | 41348409 | 41348554 | −0.0917623 | 0.0917623 |
| 1108 | 9 | 113800211 | 113800352 | −0.0915459 | 0.0915459 |
| 1109 | 9 | 119721297 | 119721565 | −0.0915334 | 0.0915334 |
| 1110 | 16 | 70284848 | 70284993 | −0.0912536 | 0.0912536 |
| 1111 | 22 | 34188961 | 34189150 | −0.0912418 | 0.0912418 |
| 1112 | 22 | 46299291 | 46299426 | −0.0910762 | 0.0910762 |
| 1113 | 14 | 66975221 | 66975373 | 0.09091819 | 0.090918193 |
| 1114 | 14 | 91575396 | 91575550 | −0.0908186 | 0.0908186 |
| 1115 | 8 | 104311272 | 104311409 | −0.0907228 | 0.0907228 |
| 1116 | 13 | 28025129 | 28025381 | 0.09053433 | 0.090534326 |
| 1117 | 18 | 10379179 | 10379396 | 0.09053103 | 0.090531027 |
| 1118 | 8 | 37887633 | 37887778 | −0.0905308 | 0.0905308 |
| 1119 | 19 | 50836522 | 50836639 | 0.09042155 | 0.090421548 |
| 1120 | 6 | 146285272 | 146285413 | 0.0901911 | 0.090191096 |
| 1121 | 6 | 164077920 | 164078085 | −0.0901833 | 0.0901833 |
| 1122 | 6 | 42858622 | 42858772 | 0.09017961 | 0.090179608 |
| 1123 | 1 | 197882278 | 197882371 | 0.09012776 | 0.090127756 |
| 1124 | 8 | 49577443 | 49577714 | 0.0899383 | 0.089938298 |
| 1125 | 12 | 108954188 | 108954345 | −0.0898893 | 0.0898893 |
| 1126 | 14 | 21081795 | 21081940 | 0.08986327 | 0.08986327 |
| 1127 | 1 | 226830883 | 226831026 | 0.08981544 | 0.089815437 |
| 1128 | 17 | 27054376 | 27054506 | 0.08968653 | 0.089686532 |
| 1129 | 16 | 1823966 | 1824109 | −0.0895844 | 0.0895844 |
| 1130 | 11 | 33037320 | 33037467 | −0.089582 | 0.089582 |
| 1131 | 17 | 26733223 | 26733445 | −0.0895731 | 0.0895731 |
| 1132 | 16 | 10946416 | 10946560 | 0.08932217 | 0.089322173 |
| 1133 | 6 | 29760330 | 29760487 | 0.08921485 | 0.089214845 |
| 1134 | 6 | 31461673 | 31461805 | 0.08921226 | 0.089212263 |
| 1135 | 2 | 45795143 | 45795285 | 0.08908486 | 0.089084861 |
| 1136 | 7 | 73021467 | 73021688 | −0.088997 | 0.088997 |
| 1137 | 1 | 17445850 | 17446097 | 0.08899651 | 0.088996515 |
| 1138 | 12 | 52345107 | 52345260 | 0.08882217 | 0.088822167 |
| 1139 | 22 | 18560560 | 18560756 | −0.088735 | 0.088735 |
| 1140 | 22 | 25800969 | 25801118 | 0.08856075 | 0.088560753 |
| 1141 | 15 | 65903246 | 65903549 | 0.08841214 | 0.088412136 |
| 1142 | 3 | 37034619 | 37034872 | −0.0884021 | 0.0884021 |
| 1143 | 18 | 49868052 | 49868196 | −0.0882821 | 0.0882821 |
| 1144 | 12 | 89745398 | 89745553 | −0.088223 | 0.088223 |
| 1145 | 19 | 33071615 | 33071751 | 0.08817966 | 0.088179664 |
| 1146 | 2 | 71132835 | 71133065 | −0.0879974 | 0.0879974 |
| 1147 | 3 | 37034268 | 37034416 | 0.08796628 | 0.087966275 |
| 1148 | 11 | 46264998 | 46265135 | −0.0879516 | 0.0879516 |
| 1149 | 6 | 37321576 | 37321756 | −0.0878804 | 0.0878804 |
| 1150 | 6 | 170151313 | 170151463 | 0.08787529 | 0.087875286 |
| 1151 | 2 | 242577080 | 242577229 | −0.0878273 | 0.0878273 |
| 1152 | 1 | 202830640 | 202830788 | 0.08777001 | 0.08777001 |
| 1153 | 1 | 94344091 | 94344241 | 0.08771363 | 0.08771363 |
| 1154 | 22 | 24093346 | 24093516 | 0.08765703 | 0.087657026 |
| 1155 | 13 | 22033074 | 22033224 | −0.0875513 | 0.0875513 |
| 1156 | 19 | 52772791 | 52772938 | 0.08752658 | 0.087526575 |
| 1157 | 5 | 96270850 | 96271406 | 0.08741236 | 0.087412364 |
| 1158 | 14 | 74220181 | 74220323 | −0.0873557 | 0.0873557 |
| 1159 | 1 | 91966897 | 91967031 | 0.08715328 | 0.087153281 |
| 1160 | 3 | 42641674 | 42641824 | −0.087074 | 0.087074 |
| 1161 | 17 | 76744733 | 76744890 | −0.0868355 | 0.0868355 |
| 1162 | 10 | 11220584 | 11220883 | −0.0868285 | 0.0868285 |
| 1163 | 1 | 21022910 | 21023059 | 0.08679873 | 0.086798728 |
| 1164 | 17 | 30264420 | 30264558 | 0.08676035 | 0.086760354 |
| 1165 | 3 | 160555464 | 160555623 | 0.08672866 | 0.086728657 |
| 1166 | 4 | 89080112 | 89080273 | 0.08667388 | 0.086673884 |
| 1167 | 3 | 49208831 | 49209075 | 0.08659727 | 0.086597273 |
| 1168 | 17 | 58678093 | 58678238 | 0.08658299 | 0.086582994 |
| 1169 | 21 | 46360274 | 46360523 | 0.08654014 | 0.086540141 |
| 1170 | 13 | 52585518 | 52585710 | −0.086501 | 0.086501 |
| 1171 | 18 | 47721153 | 47721298 | 0.08645755 | 0.086457552 |
| 1172 | 2 | 177502938 | 177503108 | 0.0864132 | 0.086413198 |
| 1173 | 1 | 161500533 | 161500755 | 0.08630859 | 0.086308585 |
| 1174 | 6 | 21587638 | 21587776 | 0.08628851 | 0.086288513 |
| 1175 | 1 | 44435536 | 44435683 | −0.0862853 | 0.0862853 |
| 1176 | 1 | 184724115 | 184724270 | −0.0862587 | 0.0862587 |
| 1177 | 13 | 98087314 | 98087462 | 0.08619268 | 0.086192679 |
| 1178 | 14 | 74226884 | 74227160 | 0.08593088 | 0.085930882 |
| 1179 | 5 | 42423998 | 42424155 | −0.0859134 | 0.0859134 |
| 1180 | 3 | 14220257 | 14220421 | 0.08578794 | 0.085787942 |
| 1181 | 1 | 227127937 | 227128110 | −0.0857376 | 0.0857376 |
| 1182 | 19 | 40931618 | 40931759 | 0.08569642 | 0.085696417 |
| 1183 | 17 | 45781653 | 45781828 | −0.0856921 | 0.0856921 |
| 1184 | 6 | 5260585 | 5260724 | 0.08555339 | 0.085553388 |
| 1185 | 12 | 7000724 | 7000872 | −0.0854119 | 0.0854119 |
| 1186 | 20 | 34207396 | 34207530 | 0.08539363 | 0.085393628 |
| 1187 | 12 | 51420267 | 51420409 | −0.0853106 | 0.0853106 |
| 1188 | 11 | 31832633 | 31832790 | −0.0851789 | 0.0851789 |
| 1189 | 2 | 26257374 | 26257515 | 0.08511268 | 0.085112675 |
| 1190 | 19 | 47363859 | 47364003 | −0.0848896 | 0.0848896 |
| 1191 | 6 | 143163603 | 143163787 | 0.08463122 | 0.084631225 |
| 1192 | 1 | 150947694 | 150947926 | −0.0845615 | 0.0845615 |
| 1193 | 14 | 52734279 | 52735795 | −0.084486 | 0.084486 |
| 1194 | 13 | 49549808 | 49549955 | −0.084431 | 0.084431 |
| 1195 | 11 | 117014646 | 117014894 | 0.08439352 | 0.084393516 |
| 1196 | 4 | 102268165 | 102268325 | 0.0843187 | 0.084318695 |
| 1197 | 2 | 152685017 | 152685311 | −0.0842916 | 0.0842916 |
| 1198 | 19 | 46996378 | 46996594 | −0.0841687 | 0.0841687 |
| 1199 | 10 | 94353703 | 94353827 | 0.0840386 | 0.084038596 |
| 1200 | 15 | 83680030 | 83680172 | −0.0836783 | 0.0836783 |
| 1201 | 14 | 64854495 | 64854665 | −0.0836314 | 0.0836314 |
| 1202 | 1 | 222791539 | 222791716 | −0.08332 | 0.08332 |
| 1203 | 16 | 2021800 | 2022155 | 0.08328386 | 0.083283856 |
| 1204 | 11 | 67252313 | 67252460 | 0.08321113 | 0.083211127 |
| 1205 | 6 | 99292714 | 99292858 | −0.083183 | 0.083183 |
| 1206 | 21 | 16437688 | 16437825 | −0.0831555 | 0.0831555 |
| 1207 | 3 | 48955944 | 48956095 | 0.0831361 | 0.083136096 |
| 1208 | 19 | 32532908 | 32533065 | 0.08298101 | 0.082981009 |
| 1209 | 19 | 55836654 | 55836810 | −0.082981 | 0.082981 |
| 1210 | 14 | 92338452 | 92338717 | −0.0829253 | 0.0829253 |
| 1211 | 3 | 194992564 | 194992710 | −0.082861 | 0.082861 |
| 1212 | 17 | 7760958 | 7761393 | 0.08279429 | 0.082794286 |
| 1213 | 1 | 714517 | 714607 | −0.0827102 | 0.0827102 |
| 1214 | 4 | 8271334 | 8271478 | −0.0826678 | 0.0826678 |
| 1215 | 15 | 78592235 | 78592340 | 0.08266035 | 0.082660352 |
| 1216 | 3 | 184099353 | 184099500 | 0.08234757 | 0.082347567 |
| 1217 | 9 | 126773802 | 126773950 | 0.08232316 | 0.082323164 |
| 1218 | 4 | 102711602 | 102712426 | 0.08230723 | 0.082307228 |
| 1219 | 7 | 26904081 | 26904262 | 0.08219771 | 0.08219771 |
| 1220 | 2 | 239288184 | 239288334 | −0.0821479 | 0.0821479 |
| 1221 | 1 | 114471726 | 114471902 | −0.0821278 | 0.0821278 |
| 1222 | 8 | 81786792 | 81786942 | 0.08210894 | 0.082108939 |
| 1223 | 17 | 4614581 | 4615022 | −0.0820247 | 0.0820247 |
| 1224 | 14 | 73392846 | 73393018 | 0.08199361 | 0.081993608 |
| 1225 | 10 | 105419967 | 105420222 | −0.0819922 | 0.0819922 |
| 1226 | 3 | 15468922 | 15469271 | 0.08194603 | 0.081946033 |
| 1227 | 11 | 62573017 | 62573167 | −0.0819262 | 0.0819262 |
| 1228 | 5 | 11384740 | 11385659 | −0.0819232 | 0.0819232 |
| 1229 | 14 | 32546870 | 32547002 | 0.08190177 | 0.081901765 |
| 1230 | 5 | 180688152 | 180688357 | −0.0818958 | 0.0818958 |
| 1231 | 6 | 31670440 | 31671422 | 0.08188283 | 0.081882832 |
| 1232 | 21 | 45209539 | 45209683 | 0.08178083 | 0.081780834 |
| 1233 | 2 | 20549696 | 20549851 | −0.0816044 | 0.0816044 |
| 1234 | 2 | 15829630 | 15829773 | 0.0816002 | 0.081600204 |
| 1235 | 4 | 57666311 | 57666455 | 0.08151527 | 0.081515274 |
| 1236 | 12 | 107487286 | 107487473 | −0.0811945 | 0.0811945 |
| 1237 | 5 | 43556284 | 43556425 | 0.08116224 | 0.081162239 |
| 1238 | 1 | 167189720 | 167189754 | 0.08111246 | 0.081112461 |
| 1239 | 11 | 104034541 | 104034818 | −0.0811106 | 0.0811106 |
| 1240 | 11 | 9635810 | 9635957 | −0.0808309 | 0.0808309 |
| 1241 | 4 | 153456067 | 153456217 | 0.08080534 | 0.080805337 |
| 1242 | 1 | 46598680 | 46598858 | −0.0807009 | 0.0807009 |
| 1243 | 2 | 63278100 | 63278255 | 0.08061692 | 0.080616922 |
| 1244 | 10 | 18947784 | 18947918 | 0.0804209 | 0.080420897 |
| 1245 | 6 | 27145497 | 27145641 | −0.0804162 | 0.0804162 |
| 1246 | 6 | 41605266 | 41605411 | −0.0804041 | 0.0804041 |
| 1247 | 17 | 6946584 | 6947623 | −0.0803045 | 0.0803045 |
| 1248 | 7 | 100887632 | 100887775 | −0.0802643 | 0.0802643 |
| 1249 | 3 | 147126883 | 147127174 | −0.0802162 | 0.0802162 |
| 1250 | 6 | 147851408 | 147851691 | −0.0801764 | 0.0801764 |
| 1251 | 1 | 70876725 | 70877040 | 0.08008784 | 0.080087843 |
| 1252 | 10 | 77846404 | 77846930 | 0.08000145 | 0.080001453 |
| 1253 | 12 | 54145351 | 54145557 | 0.07996224 | 0.079962243 |
| 1254 | 4 | 153700812 | 153700956 | −0.0799126 | 0.0799126 |
| 1255 | 10 | 104406579 | 104406716 | −0.0797627 | 0.0797627 |
| 1256 | 2 | 196521423 | 196521568 | 0.07973329 | 0.079733294 |
| 1257 | 12 | 3600588 | 3600750 | 0.07956353 | 0.079563528 |
| 1258 | 3 | 113557586 | 113557716 | −0.0795496 | 0.0795496 |
| 1259 | 1 | 87380815 | 87381029 | 0.07954435 | 0.079544345 |
| 1260 | 12 | 54360613 | 54360724 | −0.0795332 | 0.0795332 |
| 1261 | 22 | 46731152 | 46731332 | 0.07942948 | 0.079429478 |
| 1262 | 4 | 89444366 | 89444504 | 0.07937932 | 0.079379322 |
| 1263 | 1 | 204159764 | 204159894 | −0.0793083 | 0.0793083 |
| 1264 | 17 | 59494182 | 59494339 | −0.0791127 | 0.0791127 |
| 1265 | 1 | 145589294 | 145589445 | 0.07886059 | 0.078860587 |
| 1266 | 8 | 22422935 | 22423138 | −0.0788544 | 0.0788544 |
| 1267 | 3 | 13937269 | 13937420 | −0.0788094 | 0.0788094 |
| 1268 | 8 | 95274487 | 95274623 | −0.0785181 | 0.0785181 |
| 1269 | 19 | 18682386 | 18682767 | 0.07851104 | 0.078511042 |
| 1270 | 19 | 23578180 | 23578324 | −0.0784898 | 0.0784898 |
| 1271 | 1 | 109236739 | 109237017 | −0.0784117 | 0.0784117 |
| 1272 | 6 | 28642320 | 28642449 | −0.078407 | 0.078407 |
| 1273 | 3 | 168960251 | 168960503 | 0.07833761 | 0.078337608 |
| 1274 | 19 | 44099336 | 44099478 | −0.0782465 | 0.0782465 |
| 1275 | 16 | 27280354 | 27280495 | −0.0779394 | 0.0779394 |
| 1276 | 17 | 18280888 | 18281109 | −0.0778118 | 0.0778118 |
| 1277 | 4 | 13526579 | 13526731 | 0.0777712 | 0.077771202 |
| 1278 | 2 | 8825837 | 8825973 | −0.0777433 | 0.0777433 |
| 1279 | 10 | 23483693 | 23484483 | −0.0775419 | 0.0775419 |
| 1280 | 10 | 122889145 | 122889298 | −0.0775175 | 0.0775175 |
| 1281 | 15 | 89456711 | 89456857 | −0.077444 | 0.077444 |
| 1282 | 17 | 40925163 | 40925301 | 0.07737535 | 0.077375354 |
| 1283 | 2 | 64455475 | 64455625 | 0.07706262 | 0.077062624 |
| 1284 | 2 | 97627527 | 97627667 | −0.0770253 | 0.0770253 |
| 1285 | 22 | 29168428 | 29168569 | −0.0769083 | 0.0769083 |
| 1286 | 19 | 36207458 | 36207603 | 0.0767369 | 0.076736902 |
| 1287 | 2 | 236179663 | 236179798 | −0.0767153 | 0.0767153 |
| 1288 | 2 | 62132547 | 62132863 | 0.07663825 | 0.076638253 |
| 1289 | 17 | 36909232 | 36909379 | 0.07660291 | 0.076602912 |
| 1290 | 1 | 16162576 | 16162733 | 0.07658866 | 0.076588663 |
| 1291 | 1 | 11119775 | 11119946 | −0.0765865 | 0.0765865 |
| 1292 | 3 | 57789573 | 57789724 | 0.07651646 | 0.076516462 |
| 1293 | 13 | 103426207 | 103426418 | 0.07648139 | 0.076481395 |
| 1294 | 5 | 134734921 | 134735139 | −0.0764715 | 0.0764715 |
| 1295 | 9 | 134158183 | 134158333 | −0.0764573 | 0.0764573 |
| 1296 | 8 | 144350102 | 144350250 | −0.0763412 | 0.0763412 |
| 1297 | 3 | 16927876 | 16928120 | −0.0762069 | 0.0762069 |
| 1298 | 5 | 36606442 | 36606602 | 0.07619943 | 0.076199429 |
| 1299 | 12 | 100593379 | 100593526 | 0.07617957 | 0.076179573 |
| 1300 | 6 | 13487592 | 13487735 | 0.07611566 | 0.076115661 |
| 1301 | 3 | 183145875 | 183146097 | 0.07609535 | 0.076095349 |
| 1302 | 9 | 71940697 | 71940835 | 0.07597478 | 0.075974776 |
| 1303 | 3 | 97540983 | 97541109 | −0.0757112 | 0.0757112 |
| 1304 | 14 | 75078764 | 75078927 | 0.07570825 | 0.075708254 |
| 1305 | 3 | 150321760 | 150321898 | 0.07561187 | 0.075611871 |
| 1306 | 6 | 14661536 | 14661684 | 0.07545465 | 0.075454647 |
| 1307 | 6 | 74019753 | 74019891 | 0.07536742 | 0.075367421 |
| 1308 | 16 | 81348294 | 81348442 | 0.07516053 | 0.075160527 |
| 1309 | 1 | 236030963 | 236031093 | −0.0750931 | 0.0750931 |
| 1310 | 18 | 61637369 | 61637508 | 0.07508784 | 0.075087838 |
| 1311 | 19 | 55951874 | 55952001 | −0.075076 | 0.075076 |
| 1312 | 16 | 57570744 | 57570878 | 0.07505805 | 0.075058049 |
| 1313 | 12 | 12876519 | 12876759 | 0.07502004 | 0.075020036 |
| 1314 | 1 | 247267252 | 247267395 | 0.07499757 | 0.074997572 |
| 1315 | 10 | 112327204 | 112327340 | 0.07494649 | 0.07494649 |
| 1316 | 19 | 3338848 | 3338988 | −0.0749459 | 0.0749459 |
| 1317 | 6 | 87864884 | 87865131 | 0.07489821 | 0.074898208 |
| 1318 | 16 | 25268818 | 25268957 | −0.0748057 | 0.0748057 |
| 1319 | 2 | 32853401 | 32853564 | 0.07462515 | 0.074625147 |
| 1320 | 12 | 6981580 | 6981875 | −0.0746208 | 0.0746208 |
| 1321 | 14 | 24422692 | 24423315 | −0.0745034 | 0.0745034 |
| 1322 | 7 | 37590089 | 37590230 | 0.07438605 | 0.074386052 |
| 1323 | 15 | 35837770 | 35837907 | 0.07423382 | 0.074233821 |
| 1324 | 17 | 46673688 | 46674103 | −0.0742176 | 0.0742176 |
| 1325 | 19 | 51843774 | 51843930 | 0.07418882 | 0.074188822 |
| 1326 | 3 | 24563417 | 24563580 | 0.07411333 | 0.074113325 |
| 1327 | 1 | 200842676 | 200842963 | −0.0739448 | 0.0739448 |
| 1328 | 12 | 111531314 | 111531542 | −0.0739356 | 0.0739356 |
| 1329 | 17 | 30677296 | 30677444 | 0.07389726 | 0.073897261 |
| 1330 | 10 | 121301109 | 121301802 | −0.07381 | 0.07381 |
| 1331 | 2 | 135761945 | 135762086 | −0.0737898 | 0.0737898 |
| 1332 | 2 | 208491884 | 208492192 | −0.0737806 | 0.0737806 |
| 1333 | 22 | 50682903 | 50683062 | 0.07355865 | 0.073558646 |
| 1334 | 6 | 57036814 | 57036978 | −0.0733884 | 0.0 |
| 1335 | 8 | 22931963 | 22932101 | 0.0733545 | 0.073 |
| 1336 | 4 | 103481278 | 103481491 | 0.07322764 | 0.073 |
| 1337 | 15 | 50554900 | 50555064 | −0.073122 | 0. |
| 1338 | 7 | 55639050 | 55639196 | −0.0730548 | 0.0 |
| 1339 | 4 | 41215954 | 41216115 | 0.0729973 | 0.0 |
| 1340 | 6 | 80579459 | 80579613 | 0.07296546 | 0.072 |
| 1341 | 14 | 89831229 | 89831624 | −0.0729164 | 0.0 |
| 1342 | 10 | 97889980 | 97890113 | 0.07282578 | 0.072 |
| 1343 | 2 | 128644335 | 128644479 | −0.0727805 | 0.0 |
| 1344 | 4 | 76650592 | 76650730 | −0.0727216 | 0.0 |
| 1345 | 1 | 117452805 | 117452987 | 0.0726993 | 0.072 |
| 1346 | 12 | 49463645 | 49463982 | 0.07265306 | 0.072 |
| 1347 | 1 | 6761856 | 6762004 | −0.072647 | 0. |
| 1348 | 20 | 591008 | 591151 | 0.07257658 | 0.072 |
| 1349 | 14 | 51706897 | 51707062 | −0.0725503 | 0.0 |
| 1350 | 4 | 40057465 | 40057613 | 0.07251481 | 0.072 |
| 1351 | 22 | 38902212 | 38902347 | −0.0724393 | 0.0 |
| 1352 | 17 | 40245148 | 40245292 | 0.07240992 | 0.072 |
| 1353 | 1 | 22109101 | 22109243 | −0.0723371 | 0.0 |
| 1354 | 4 | 87515625 | 87515761 | 0.07217381 | 0.072 |
| 1355 | 15 | 68723612 | 68723763 | −0.0721286 | 0.0 |
| 1356 | X | 49047853 | 49048396 | 0.07197285 | 0.071 |
| 1357 | 6 | 27100970 | 27101202 | 0.07195895 | 0.071 |
| 1358 | 12 | 116996844 | 116997780 | −0.0718508 | 0.0 |
| 1359 | 4 | 57301925 | 57302170 | −0.0717514 | 0.0 |
| 1360 | 10 | 112263912 | 112264045 | −0.0717406 | 0.0 |
| 1361 | 10 | 99205875 | 99206092 | −0.0715996 | 0.0 |
| 1362 | 2 | 99771346 | 99771690 | 0.07158903 | 0.071 |
| 1363 | 9 | 95055972 | 95056123 | 0.07154495 | 0.071 |
| 1364 | 17 | 29421726 | 29421972 | 0.07149059 | 0.071 |
| 1365 | 3 | 10028452 | 10028598 | −0.0714369 | 0.0 |
| 1366 | 15 | 101419574 | 101419718 | −0.0714185 | 0.0 |
| 1367 | 10 | 14223322 | 14223495 | 0.0712683 | 0.071 |
| 1368 | 10 | 94449911 | 94450857 | −0.0712111 | 0.0 |
| 1369 | 4 | 119274112 | 119274267 | 0.07103463 | 0.071034633 |
| 1370 | 15 | 75660896 | 75661094 | −0.070785 | 0.070785 |
| 1371 | 19 | 39109941 | 39110086 | −0.0706344 | 0.0706344 |
| 1372 | 12 | 4386051 | 4386178 | 0.07062279 | 0.070622794 |
| 1373 | 13 | 25321655 | 25321816 | −0.0703692 | 0.0703692 |
| 1374 | 10 | 105991883 | 105992009 | 0.07035892 | 0.070358916 |
| 1375 | 19 | 17420651 | 17420682 | −0.070248 | 0.070248 |
| 1376 | 15 | 74219591 | 74219739 | −0.0700628 | 0.0700628 |
| 1377 | 12 | 54343765 | 54343913 | 0.07002518 | 0.070025185 |
| 1378 | 19 | 18499231 | 18499376 | 0.07000354 | 0.070003544 |
| 1379 | 11 | 1968593 | 1968710 | −0.0699194 | 0.0699194 |
| 1380 | 12 | 104852073 | 104852226 | 0.06982769 | 0.069827692 |
| 1381 | 11 | 35165773 | 35166045 | 0.06973297 | 0.069732974 |
| 1382 | 19 | 2819565 | 2819841 | −0.069697 | 0.069697 |
| 1383 | 14 | 103851423 | 103851669 | −0.06965 | 0.06965 |
| 1384 | 1 | 154971854 | 154971970 | 0.0695939 | 0.069593897 |
| 1385 | 4 | 87813207 | 87813356 | 0.06955204 | 0.069552038 |
| 1386 | 14 | 91783818 | 91784518 | −0.0692192 | 0.0692192 |
| 1387 | 11 | 48034961 | 48035251 | −0.069207 | 0.069207 |
| 1388 | 19 | 18118756 | 18118842 | 0.06916798 | 0.069167985 |
| 1389 | 15 | 57622285 | 57622434 | 0.06914836 | 0.069148364 |
| 1390 | 1 | 38273262 | 38273463 | −0.0690915 | 0.0690915 |
| 1391 | 3 | 49141965 | 49142113 | 0.0690659 | 0.069065903 |
| 1392 | 17 | 78388814 | 78389133 | 0.06903137 | 0.069031368 |
| 1393 | 10 | 70231549 | 70231712 | 0.06896393 | 0.068963935 |
| 1394 | 16 | 88521778 | 88521932 | −0.0688131 | 0.0688131 |
| 1395 | 10 | 95242254 | 95242390 | −0.0687492 | 0.0687492 |
| 1396 | 14 | 37051793 | 37051938 | 0.06868124 | 0.068681244 |
| 1397 | 14 | 100438657 | 100438798 | −0.0686544 | 0.0686544 |
| 1398 | 11 | 18655840 | 18656245 | −0.0685289 | 0.0685289 |
| 1399 | 2 | 97405731 | 97405947 | −0.0685107 | 0.0685107 |
| 1400 | 1 | 27669755 | 27670067 | −0.0683932 | 0.0683932 |
| 1401 | 15 | 40453211 | 40453385 | 0.06836581 | 0.068365806 |
| 1402 | 5 | 157023552 | 157023746 | 0.06833194 | 0.068331938 |
| 1403 | 22 | 24237264 | 24237408 | −0.0682984 | 0.0682984 |
| 1404 | 8 | 144373377 | 144373595 | −0.0682672 | 0.0682672 |
| 1405 | 17 | 48074346 | 48074503 | 0.06826281 | 0.068262812 |
| 1406 | 2 | 101790440 | 101790565 | −0.0681967 | 0.0681967 |
| 1407 | 2 | 61244765 | 61244913 | −0.0681426 | 0.0681426 |
| 1408 | 17 | 47928769 | 47928861 | 0.06791348 | 0.067913477 |
| 1409 | 4 | 47465502 | 47465853 | −0.0678742 | 0.0678742 |
| 1410 | 1 | 19923766 | 19923920 | −0.0676718 | 0.0676718 |
| 1411 | 1 | 154375671 | 154376283 | −0.0676458 | 0.0676458 |
| 1412 | 5 | 153990888 | 153991036 | −0.0676358 | 0.0676358 |
| 1413 | 14 | 52535987 | 52536124 | −0.0674196 | 0.0674196 |
| 1414 | 13 | 40781078 | 40781257 | 0.06738241 | 0.067382406 |
| 1415 | 1 | 228633381 | 228633530 | 0.06734305 | 0.067343053 |
| 1416 | 11 | 131780299 | 131781399 | −0.0673332 | 0.0673332 |
| 1417 | 16 | 30075119 | 30075610 | −0.0672909 | 0.0672909 |
| 1418 | 6 | 34113220 | 34113367 | 0.06717851 | 0.067178513 |
| 1419 | 1 | 8938927 | 8939206 | 0.067119 | 0.067118996 |
| 1420 | 15 | 65117377 | 65117525 | −0.0670929 | 0.0670929 |
| 1421 | 20 | 52488881 | 52489048 | 0.06707789 | 0.067077889 |
| 1422 | 18 | 47476431 | 47476668 | 0.06704155 | 0.067041552 |
| 1423 | 16 | 29611434 | 29611909 | −0.0669547 | 0.0669547 |
| 1424 | 19 | 41257355 | 41257504 | 0.06693737 | 0.066937369 |
| 1425 | 6 | 11224737 | 11224871 | 0.06693137 | 0.066931372 |
| 1426 | 7 | 138915960 | 138916100 | 0.06692137 | 0.066921367 |
| 1427 | 1 | 52456341 | 52456513 | −0.0669146 | 0.0669146 |
| 1428 | 15 | 88579785 | 88579927 | −0.0668697 | 0.0668697 |
| 1429 | 19 | 45157649 | 45157795 | −0.0668643 | 0.0668643 |
| 1430 | 6 | 44494825 | 44494964 | −0.0668499 | 0.0668499 |
| 1431 | 3 | 47020893 | 47021036 | 0.06684119 | 0.066841193 |
| 1432 | 4 | 7710808 | 7710944 | 0.06683578 | 0.066835783 |
| 1433 | 4 | 88343927 | 88344073 | −0.0668289 | 0.0668289 |
| 1434 | 8 | 23584018 | 23584162 | 0.06676321 | 0.066763212 |
| 1435 | 3 | 169940113 | 169940272 | −0.0667524 | 0.0667524 |
| 1436 | 20 | 3452207 | 3452362 | −0.0667246 | 0.0667246 |
| 1437 | 16 | 30960403 | 30960554 | −0.0666995 | 0.0666995 |
| 1438 | 8 | 37757195 | 37757317 | −0.0664322 | 0.0664322 |
| 1439 | 19 | 1383624 | 1383984 | 0.06631448 | 0.066314481 |
| 1440 | 3 | 169377681 | 169378040 | −0.0662975 | 0.0662975 |
| 1441 | 17 | 7232691 | 7232948 | −0.066283 | 0.066283 |
| 1442 | 1 | 55266859 | 55266999 | −0.0662448 | 0.0662448 |
| 1443 | 11 | 94474045 | 94474717 | −0.0661167 | 0.0661167 |
| 1444 | 4 | 148721044 | 148721603 | −0.0661107 | 0.0661107 |
| 1445 | 11 | 64808292 | 64808575 | −0.0660504 | 0.0660504 |
| 1446 | 11 | 112096801 | 112096929 | −0.0660117 | 0.0660117 |
| 1447 | 7 | 98741519 | 98741656 | −0.0659721 | 0.0659721 |
| 1448 | 4 | 100009822 | 100009952 | −0.0659712 | 0.0659712 |
| 1449 | 16 | 791310 | 791447 | −0.0659366 | 0.0659366 |
| 1450 | 2 | 233947203 | 233947392 | −0.0659364 | 0.0659364 |
| 1451 | 10 | 70092744 | 70092889 | 0.06584639 | 0.065846387 |
| 1452 | 14 | 69283406 | 69283585 | 0.06582142 | 0.065821424 |
| 1453 | 1 | 145477154 | 145477274 | −0.0657689 | 0.0657689 |
| 1454 | 19 | 13001817 | 13001956 | −0.0657291 | 0.0657291 |
| 1455 | 7 | 129592241 | 129592408 | −0.065703 | 0.065703 |
| 1456 | 12 | 57634390 | 57634541 | 0.06569519 | 0.065695188 |
| 1457 | 16 | 56691770 | 56691914 | 0.06564482 | 0.065644825 |
| 1458 | 12 | 124086116 | 124086557 | 0.06558476 | 0.065584755 |
| 1459 | 16 | 10479887 | 10480027 | 0.06549719 | 0.065497188 |
| 1460 | 6 | 160490263 | 160490477 | −0.06547 | 0.06547 |
| 1461 | 5 | 166405269 | 166405399 | −0.0654011 | 0.0654011 |
| 1462 | 22 | 46367390 | 46368565 | −0.0653565 | 0.0653565 |
| 1463 | 6 | 43395333 | 43395468 | 0.06530154 | 0.065301544 |
| 1464 | 11 | 61284310 | 61284485 | 0.06527909 | 0.065279092 |
| 1465 | 18 | 11980610 | 11980763 | 0.06518034 | 0.065180336 |
| 1466 | 17 | 46125546 | 46125696 | −0.065097 | 0.065097 |
| 1467 | 2 | 29117656 | 29117889 | 0.06500599 | 0.065005989 |
| 1468 | 1 | 147013506 | 147013652 | −0.0648812 | 0.0648812 |
| 1469 | 19 | 36239832 | 36239959 | −0.064862 | 0.064862 |
| 1470 | 13 | 41635441 | 41635570 | 0.06481267 | 0.064812671 |
| 1471 | 13 | 30880753 | 30880917 | −0.0647782 | 0.0647782 |
| 1472 | 3 | 55040461 | 55040643 | −0.0647187 | 0.0647187 |
| 1473 | 1 | 168148473 | 168148601 | 0.06446946 | 0.064469459 |
| 1474 | 2 | 153032607 | 153032820 | 0.06442883 | 0.064428835 |
| 1475 | 12 | 49110134 | 49110278 | −0.0643318 | 0.0643318 |
| 1476 | 7 | 5534600 | 5534820 | −0.0643042 | 0.0643042 |
| 1477 | 12 | 55413804 | 55413942 | −0.0642967 | 0.0642967 |
| 1478 | 7 | 23510055 | 23510195 | 0.06427013 | 0.064270126 |
| 1479 | 2 | 97505000 | 97505608 | −0.064171 | 0.064171 |
| 1480 | 1 | 161123619 | 161123901 | 0.06414092 | 0.064140922 |
| 1481 | 5 | 16617551 | 16617694 | −0.0640359 | 0.0640359 |
| 1482 | 10 | 58120928 | 58121244 | 0.0639121 | 0.063912099 |
| 1483 | 6 | 118971612 | 118971745 | −0.0639017 | 0.0639017 |
| 1484 | 4 | 113437829 | 113437989 | 0.06383491 | 0.063834909 |
| 1485 | 12 | 7125646 | 7125792 | −0.0633499 | 0.0633499 |
| 1486 | 8 | 99985833 | 99987014 | −0.0632642 | 0.0632642 |
| 1487 | 15 | 75075976 | 75076565 | −0.0632302 | 0.0632302 |
| 1488 | 3 | 139048259 | 139048406 | 0.0631244 | 0.063124402 |
| 1489 | 19 | 51014582 | 51014719 | 0.0630424 | 0.063042401 |
| 1490 | 10 | 116527572 | 116527726 | 0.06290597 | 0.062905967 |
| 1491 | 12 | 104235231 | 104235373 | −0.0628491 | 0.0628491 |
| 1492 | X | 149531950 | 149532201 | 0.06283204 | 0.062832044 |
| 1493 | 1 | 57254349 | 57254489 | −0.0627992 | 0.0627992 |
| 1494 | 3 | 182511012 | 182511139 | 0.06277676 | 0.062776764 |
| 1495 | 11 | 61735234 | 61735403 | −0.0627222 | 0.0627222 |
| 1496 | 13 | 112710714 | 112711663 | −0.0625942 | 0.0625942 |
| 1497 | 19 | 42681346 | 42681498 | 0.06246043 | 0.062460426 |
| 1498 | 8 | 59572375 | 59572552 | −0.0624521 | 0.0624521 |
| 1499 | 10 | 33269699 | 33269875 | 0.06225773 | 0.062257734 |
| 1500 | 20 | 55787850 | 55788043 | 0.06222493 | 0.062224925 |
| 1501 | 2 | 43445592 | 43445931 | −0.0621391 | 0.0621391 |
| 1502 | 15 | 89950225 | 89950761 | −0.0621284 | 0.0621284 |
| 1503 | 16 | 67217680 | 67217827 | 0.06211676 | 0.062116763 |
| 1504 | 19 | 57999386 | 57999501 | 0.06205831 | 0.062058309 |
| 1505 | 1 | 155278199 | 155278335 | −0.0620558 | 0.0620558 |
| 1506 | 22 | 27067579 | 27067792 | −0.0619655 | 0.0619655 |
| 1507 | 14 | 89895934 | 89896115 | 0.06192872 | 0.061928722 |
| 1508 | 12 | 89746253 | 89746389 | 0.0619131 | 0.061913097 |
| 1509 | 10 | 98956099 | 98956244 | 0.06187832 | 0.061878323 |
| 1510 | 5 | 40680003 | 40680093 | −0.061876 | 0.061876 |
| 1511 | 7 | 155137852 | 155137985 | −0.0617756 | 0.0617756 |
| 1512 | 11 | 93862639 | 93862812 | 0.06170645 | 0.06170645 |
| 1513 | 9 | 132598178 | 132598321 | −0.0616729 | 0.0616729 |
| 1514 | 2 | 89064640 | 89065095 | −0.0616638 | 0.0616638 |
| 1515 | 17 | 37824484 | 37824589 | −0.0616066 | 0.0616066 |
| 1516 | 15 | 42186980 | 42187305 | −0.0615827 | 0.0615827 |
| 1517 | 1 | 32538485 | 32538628 | 0.06149362 | 0.061493619 |
| 1518 | 14 | 75084789 | 75085100 | 0.0614399 | 0.0614399 |
| 1519 | 1 | 232929727 | 232929868 | −0.0613728 | 0.0613728 |
| 1520 | 16 | 57279565 | 57279729 | 0.06136956 | 0.061369556 |
| 1521 | 22 | 29137828 | 29138151 | 0.06123253 | 0.061232534 |
| 1522 | 14 | 24911937 | 24912163 | 0.06117432 | 0.061174317 |
| 1523 | 10 | 99789935 | 99790756 | −0.0611623 | 0.0611623 |
| 1524 | 21 | 44073847 | 44073986 | 0.06112069 | 0.061120687 |
| 1525 | 19 | 6361883 | 6362006 | 0.0611063 | 0.061106299 |
| 1526 | 2 | 181846507 | 181846646 | −0.0610954 | 0.0610954 |
| 1527 | 6 | 127664768 | 127664919 | 0.06104096 | 0.061040955 |
| 1528 | 17 | 61554027 | 61554173 | 0.06101625 | 0.061016252 |
| 1529 | 2 | 10549451 | 10549582 | −0.0610147 | 0.0610147 |
| 1530 | 18 | 60986688 | 60986838 | 0.06100925 | 0.061009253 |
| 1531 | 1 | 6639860 | 6640034 | −0.0609881 | 0.0609881 |
| 1532 | 7 | 45066626 | 45067088 | 0.06094465 | 0.060944647 |
| 1533 | 11 | 126081564 | 126081878 | 0.06091442 | 0.060914417 |
| 1534 | 9 | 137030175 | 137030327 | 0.06085835 | 0.060858353 |
| 1535 | 15 | 59980190 | 59980339 | −0.0608286 | 0.0608286 |
| 1536 | 15 | 34875041 | 34875175 | 0.06082396 | 0.060823961 |
| 1537 | 13 | 95655063 | 95655497 | −0.0607597 | 0.0607597 |
| 1538 | 20 | 61810555 | 61810684 | 0.06067127 | 0.060671265 |
| 1539 | 10 | 60145015 | 60145224 | −0.0606086 | 0.0606086 |
| 1540 | 2 | 30454264 | 30454422 | −0.0605935 | 0.0605935 |
| 1541 | 11 | 47381227 | 47381407 | −0.0605769 | 0.0605769 |
| 1542 | 2 | 75427985 | 75428196 | −0.0605755 | 0.0605755 |
| 1543 | 15 | 50474521 | 50474663 | −0.0604858 | 0.0604858 |
| 1544 | 11 | 33279932 | 33280085 | 0.06036745 | 0.060367447 |
| 1545 | 5 | 180230246 | 180231497 | −0.060325 | 0.060325 |
| 1546 | 11 | 18035056 | 18035206 | 0.06024565 | 0.060245648 |
| 1547 | 16 | 65156658 | 65157090 | −0.0601628 | 0.0601628 |
| 1548 | 21 | 45148112 | 45148427 | 0.06012409 | 0.060124088 |
| 1549 | 10 | 71390175 | 71390316 | −0.0601115 | 0.0601115 |
| 1550 | 6 | 27106994 | 27107870 | −0.060035 | 0.060035 |
| 1551 | 14 | 89506798 | 89506930 | −0.0599977 | 0.0599977 |
| 1552 | 2 | 129508030 | 129508175 | 0.05982527 | 0.059825273 |
| 1553 | 3 | 48936311 | 48936554 | −0.059717 | 0.059717 |
| 1554 | 13 | 92051323 | 92051474 | 0.05963122 | 0.059631222 |
| 1555 | 21 | 35987459 | 35987590 | −0.0596238 | 0.0596238 |
| 1556 | 8 | 124252890 | 124253048 | 0.05958382 | 0.059583823 |
| 1557 | 20 | 55964451 | 55964597 | 0.05950828 | 0.059508275 |
| 1558 | 5 | 110560294 | 110560437 | 0.05945218 | 0.059452178 |
| 1559 | 2 | 235459223 | 235459362 | −0.0594435 | 0.0594435 |
| 1560 | 21 | 35747507 | 35747644 | −0.0594037 | 0.0594037 |
| 1561 | 11 | 72504633 | 72504788 | −0.0593953 | 0.0593953 |
| 1562 | 12 | 130388930 | 130389067 | −0.0593298 | 0.0593298 |
| 1563 | 19 | 45579302 | 45579435 | 0.05932751 | 0.059327506 |
| 1564 | 3 | 43732494 | 43732636 | 0.05922118 | 0.059221184 |
| 1565 | 8 | 87520663 | 87520805 | 0.05907619 | 0.05907619 |
| 1566 | 6 | 167040360 | 167040510 | −0.0589265 | 0.0589265 |
| 1567 | 12 | 57504310 | 57504443 | 0.05891256 | 0.058912559 |
| 1568 | 18 | 60986863 | 60987001 | 0.05890108 | 0.058901077 |
| 1569 | 7 | 23571241 | 23571392 | −0.0588981 | 0.0588981 |
| 1570 | 8 | 145754148 | 145754250 | −0.058885 | 0.058885 |
| 1571 | 1 | 208084337 | 208084516 | 0.05884876 | 0.058848761 |
| 1572 | 19 | 3180481 | 3181255 | −0.0587831 | 0.0587831 |
| 1573 | 22 | 43583663 | 43583821 | −0.0587617 | 0.0587617 |
| 1574 | 16 | 15108232 | 15108380 | −0.0587198 | 0.0587198 |
| 1575 | 17 | 48050207 | 48050425 | 0.05863902 | 0.058639024 |
| 1576 | 13 | 114890484 | 114890644 | 0.05863659 | 0.058636588 |
| 1577 | 3 | 99979773 | 99979931 | 0.05862996 | 0.058629958 |
| 1578 | 17 | 4843079 | 4843220 | 0.05856322 | 0.058563223 |
| 1579 | 1 | 1510646 | 1510789 | 0.05849756 | 0.058497557 |
| 1580 | 16 | 75600637 | 75600783 | 0.05848879 | 0.058488791 |
| 1581 | 1 | 154934054 | 154934185 | −0.0583299 | 0.0583299 |
| 1582 | 6 | 139349517 | 139349661 | −0.058297 | 0.058297 |
| 1583 | 17 | 66940807 | 66940987 | −0.0582659 | 0.0582659 |
| 1584 | 3 | 122512884 | 122513025 | 0.05822021 | 0.05822021 |
| 1585 | 17 | 899713 | 899863 | 0.05817084 | 0.058170845 |
| 1586 | 19 | 45927616 | 45927762 | −0.0581346 | 0.0581346 |
| 1587 | 19 | 58258567 | 58258710 | −0.058114 | 0.058114 |
| 1588 | 17 | 56084290 | 56084433 | −0.0580994 | 0.0580994 |
| 1589 | 6 | 35656169 | 35656311 | −0.0580834 | 0.0580834 |
| 1590 | 12 | 109490252 | 109490470 | 0.0580538 | 0.058053805 |
| 1591 | 5 | 66299802 | 66299942 | 0.05797762 | 0.057977622 |
| 1592 | 7 | 112758655 | 112758858 | 0.05792169 | 0.057921692 |
| 1593 | 10 | 11615801 | 11615955 | −0.0578798 | 0.0578798 |
| 1594 | 6 | 11236686 | 11236862 | −0.0578435 | 0.0578435 |
| 1595 | 4 | 1341497 | 1341658 | 0.05782508 | 0.057825083 |
| 1596 | 1 | 23857242 | 23857387 | −0.0578202 | 0.0578202 |
| 1597 | X | 765772 | 765908 | 0.05779822 | 0.057798219 |
| 1598 | 19 | 1095351 | 1095599 | 0.05779319 | 0.057793191 |
| 1599 | 12 | 50451017 | 50451168 | 0.05776348 | 0.057763483 |
| 1600 | 9 | 88969686 | 88969840 | 0.05764064 | 0.057640639 |
| 1601 | 8 | 33342496 | 33342858 | 0.05759447 | 0.057594475 |
| 1602 | 15 | 72668037 | 72668219 | −0.0575399 | 0.0575399 |
| 1603 | 1 | 167598904 | 167599397 | −0.0575231 | 0.0575231 |
| 1604 | 19 | 37569164 | 37569470 | 0.05751998 | 0.057519983 |
| 1605 | 5 | 7850125 | 7850277 | 0.05746799 | 0.057467988 |
| 1606 | 7 | 20698298 | 20698471 | 0.05739104 | 0.057391044 |
| 1607 | 6 | 99283481 | 99283623 | −0.0573733 | 0.0573733 |
| 1608 | 10 | 121632983 | 121633141 | 0.05733661 | 0.057336607 |
| 1609 | 5 | 36606715 | 36606871 | 0.05731299 | 0.05731299 |
| 1610 | 12 | 88536062 | 88536226 | −0.0572492 | 0.0572492 |
| 1611 | 16 | 50781174 | 50781359 | −0.0572438 | 0.0572438 |
| 1612 | 17 | 79825005 | 79825153 | −0.0572106 | 0.0572106 |
| 1613 | 16 | 72698774 | 72698921 | −0.0571735 | 0.0571735 |
| 1614 | 15 | 40074938 | 40075265 | −0.0571484 | 0.0571484 |
| 1615 | 9 | 5627433 | 5627595 | −0.0570234 | 0.0570234 |
| 1616 | 11 | 20035502 | 20035638 | −0.0570019 | 0.0570019 |
| 1617 | 17 | 17585698 | 17585841 | −0.0569979 | 0.0569979 |
| 1618 | 1 | 202318007 | 202318154 | 0.05697674 | 0.056976736 |
| 1619 | 10 | 90967036 | 90967246 | 0.05697434 | 0.056974345 |
| 1620 | 8 | 110346474 | 110346750 | 0.05696992 | 0.056969921 |
| 1621 | 1 | 860850 | 861026 | 0.05692402 | 0.056924022 |
| 1622 | 2 | 152493809 | 152494159 | −0.0568369 | 0.0568369 |
| 1623 | 6 | 30028864 | 30029383 | 0.05678301 | 0.056783009 |
| 1624 | 16 | 20817531 | 20817749 | −0.0567202 | 0.0567202 |
| 1625 | 6 | 26331677 | 26331815 | 0.05662644 | 0.056626441 |
| 1626 | 20 | 61847505 | 61847790 | 0.05654593 | 0.05654593 |
| 1627 | 19 | 20011648 | 20011854 | 0.05643582 | 0.056435825 |
| 1628 | 5 | 36151920 | 36152311 | 0.05642614 | 0.056426138 |
| 1629 | 1 | 149858238 | 149858488 | −0.056396 | 0.056396 |
| 1630 | 1 | 84543298 | 84543453 | −0.056371 | 0.056371 |
| 1631 | 17 | 74582295 | 74582440 | 0.05628964 | 0.056289637 |
| 1632 | 15 | 86125911 | 86126313 | −0.0562493 | 0.0562493 |
| 1633 | 3 | 44037051 | 44037198 | 0.05623676 | 0.056236757 |
| 1634 | 2 | 74642057 | 74642406 | −0.056168 | 0.056168 |
| 1635 | 3 | 53925961 | 53926352 | −0.0561398 | 0.0561398 |
| 1636 | 8 | 70745388 | 70745535 | 0.0561065 | 0.056106499 |
| 1637 | 12 | 125399841 | 125399957 | 0.0561043 | 0.056104302 |
| 1638 | 17 | 79860370 | 79860490 | −0.0560787 | 0.0560787 |
| 1639 | 1 | 165667994 | 165668133 | 0.05604794 | 0.056047945 |
| 1640 | 2 | 170218624 | 170218758 | 0.05599721 | 0.055997208 |
| 1641 | 6 | 20023978 | 20024230 | −0.0559537 | 0.0559537 |
| 1642 | 8 | 134510859 | 134511407 | −0.055895 | 0.055895 |
| 1643 | 18 | 77711924 | 77712063 | 0.05583545 | 0.055835452 |
| 1644 | 22 | 43627077 | 43627209 | 0.05580591 | 0.055805909 |
| 1645 | 7 | 101458663 | 101458804 | 0.05578124 | 0.055781241 |
| 1646 | 12 | 94495835 | 94495976 | 0.0557581 | 0.0557581 |
| 1647 | 4 | 6893925 | 6894395 | −0.0557058 | 0.0557058 |
| 1648 | 3 | 147110590 | 147110731 | −0.0556217 | 0.0556217 |
| 1649 | 6 | 157801815 | 157801963 | 0.0556182 | 0.055618198 |
| 1650 | 6 | 87041199 | 87041345 | 0.05555935 | 0.055559349 |
| 1651 | 11 | 70507737 | 70508689 | −0.0555171 | 0.0555171 |
| 1652 | 15 | 41836365 | 41836646 | 0.05542046 | 0.05542046 |
| 1653 | 6 | 6002160 | 6003716 | −0.0554149 | 0.0554149 |
| 1654 | 1 | 184836671 | 184836813 | −0.0554078 | 0.0554078 |
| 1655 | 6 | 122721213 | 122721361 | −0.0554031 | 0.0554031 |
| 1656 | 8 | 145133335 | 145133611 | 0.0553189 | 0.055318902 |
| 1657 | 1 | 40974583 | 40974726 | 0.05528118 | 0.05528118 |
| 1658 | 1 | 41469262 | 41469672 | −0.0552342 | 0.0552342 |
| 1659 | 12 | 2986298 | 2986510 | −0.0552003 | 0.0552003 |
| 1660 | 15 | 90294392 | 90294671 | −0.0551672 | 0.0551672 |
| 1661 | 3 | 52740247 | 52740396 | 0.05512558 | 0.055125585 |
| 1662 | 13 | 52733487 | 52733744 | 0.05510696 | 0.055106964 |
| 1663 | 11 | 47176795 | 47176979 | −0.0550461 | 0.0550461 |
| 1664 | 10 | 105156096 | 105156517 | 0.05498357 | 0.054983574 |
| 1665 | 6 | 37470109 | 37470276 | 0.05496202 | 0.054962019 |
| 1666 | 8 | 101572018 | 101572157 | −0.0548727 | 0.0548727 |
| 1667 | 18 | 45958529 | 45958667 | −0.0547875 | 0.0547875 |
| 1668 | 2 | 25265064 | 25265231 | −0.0547664 | 0.0547664 |
| 1669 | 6 | 74363377 | 74363510 | 0.0547476 | 0.054747605 |
| 1670 | 14 | 73493866 | 73494124 | 0.05464152 | 0.054641522 |
| 1671 | 7 | 76178877 | 76179019 | −0.0546412 | 0.0546412 |
| 1672 | 16 | 68451666 | 68451820 | −0.0545943 | 0.0545943 |
| 1673 | 19 | 42901229 | 42901406 | 0.05459236 | 0.054592364 |
| 1674 | 19 | 47615915 | 47616062 | −0.0545874 | 0.0545874 |
| 1675 | 1 | 156627381 | 156627469 | −0.0545814 | 0.0545814 |
| 1676 | 9 | 131873586 | 131873726 | 0.0545706 | 0.054570597 |
| 1677 | 14 | 23236386 | 23236526 | 0.05456004 | 0.054560036 |
| 1678 | 3 | 107242554 | 107242704 | −0.0545585 | 0.0545585 |
| 1679 | 6 | 32940232 | 32940647 | 0.05452995 | 0.054529945 |
| 1680 | 8 | 97247765 | 97247970 | 0.0544437 | 0.054443695 |
| 1681 | 7 | 16684673 | 16684816 | −0.0544289 | 0.0544289 |
| 1682 | 11 | 72865504 | 72865776 | 0.05437293 | 0.054372935 |
| 1683 | 22 | 40766477 | 40766755 | 0.05431736 | 0.054317358 |
| 1684 | 6 | 114178123 | 114178299 | 0.05426821 | 0.054268209 |
| 1685 | 1 | 173684316 | 173684630 | 0.0542459 | 0.054245897 |
| 1686 | 12 | 24715423 | 24715567 | 0.05411501 | 0.054115012 |
| 1687 | 12 | 57984845 | 57985146 | 0.05410012 | 0.054100121 |
| 1688 | 6 | 113371566 | 113371782 | 0.05409353 | 0.054093525 |
| 1689 | 18 | 75362843 | 75363007 | 0.05403457 | 0.054034572 |
| 1690 | 15 | 28344269 | 28344598 | 0.05396301 | 0.053963012 |
| 1691 | 4 | 157786825 | 157787067 | 0.0539279 | 0.053927899 |
| 1692 | 19 | 3572330 | 3572479 | 0.05384364 | 0.053843638 |
| 1693 | 2 | 168149913 | 168150059 | −0.0538247 | 0.0538247 |
| 1694 | 15 | 71704619 | 71704761 | 0.05374074 | 0.053740736 |
| 1695 | 19 | 14586732 | 14586883 | −0.0536693 | 0.0536693 |
| 1696 | 8 | 125551253 | 125551431 | 0.05365092 | 0.053650924 |
| 1697 | 1 | 32226619 | 32226758 | 0.05363488 | 0.053634878 |
| 1698 | 6 | 163881680 | 163881820 | −0.0535889 | 0.0535889 |
| 1699 | 3 | 186501960 | 186502101 | −0.053513 | 0.053513 |
| 1700 | 3 | 183977474 | 183977582 | 0.0534942 | 0.053494204 |
| 1701 | 4 | 109541884 | 109542125 | −0.0534631 | 0.0534631 |
| 1702 | 5 | 126084022 | 126084156 | −0.0534541 | 0.0534541 |
| 1703 | 1 | 168105312 | 168105475 | 0.05335896 | 0.053358958 |
| 1704 | 17 | 74267918 | 74268964 | 0.05332071 | 0.053320707 |
| 1705 | X | 11446117 | 11446272 | 0.05318781 | 0.053187812 |
| 1706 | 8 | 81490300 | 81490503 | −0.0531785 | 0.0531785 |
| 1707 | 1 | 59250782 | 59251021 | 0.05317388 | 0.05317388 |
| 1708 | 8 | 58906967 | 58907118 | −0.0530744 | 0.0530744 |
| 1709 | 2 | 190445103 | 190445283 | −0.0530463 | 0.0530463 |
| 1710 | 10 | 13628773 | 13628949 | 0.05299719 | 0.052997195 |
| 1711 | 12 | 46612144 | 46612405 | 0.05293882 | 0.052938822 |
| 1712 | 16 | 66254721 | 66254969 | 0.05279505 | 0.052795049 |
| 1713 | 1 | 202780430 | 202780562 | 0.05270957 | 0.052709571 |
| 1714 | 8 | 22526242 | 22526395 | 0.05269601 | 0.052696007 |
| 1715 | 17 | 48047018 | 48047275 | −0.0526755 | 0.0526755 |
| 1716 | 2 | 98280463 | 98280923 | 0.05263864 | 0.052638639 |
| 1717 | 17 | 65713795 | 65714122 | −0.0526292 | 0.0526292 |
| 1718 | 15 | 40650175 | 40650316 | 0.05258616 | 0.052586164 |
| 1719 | 1 | 155715434 | 155715591 | −0.0525107 | 0.0525107 |
| 1720 | 16 | 791158 | 791307 | −0.0525064 | 0.0525064 |
| 1721 | 15 | 67145778 | 67145924 | 0.05247133 | 0.05247133 |
| 1722 | 10 | 32735309 | 32735448 | −0.0522241 | 0.0522241 |
| 1723 | 1 | 228675887 | 228675965 | −0.0521511 | 0.0521511 |
| 1724 | 20 | 36531269 | 36531394 | 0.05210487 | 0.052104868 |
| 1725 | 2 | 74725131 | 74725277 | 0.05204938 | 0.052049381 |
| 1726 | 1 | 168147896 | 168148445 | 0.05203756 | 0.052037561 |
| 1727 | 4 | 114684102 | 114684266 | −0.051995 | 0.051995 |
| 1728 | 7 | 2395050 | 2395226 | 0.05198374 | 0.051983742 |
| 1729 | 9 | 100880711 | 100881134 | −0.0519557 | 0.0519557 |
| 1730 | 16 | 1525089 | 1525345 | −0.0518618 | 0.0518618 |
| 1731 | 3 | 24719619 | 24719807 | −0.0517329 | 0.0517329 |
| 1732 | 7 | 5371130 | 5371312 | 0.05163402 | 0.051634022 |
| 1733 | 16 | 67282745 | 67282925 | −0.0516322 | 0.0516322 |
| 1734 | 12 | 102270817 | 102270956 | 0.05162855 | 0.051628547 |
| 1735 | 2 | 180871564 | 180871876 | −0.051596 | 0.051596 |
| 1736 | 4 | 141348782 | 141348927 | 0.05156276 | 0.051562764 |
| 1737 | 3 | 185826399 | 185826575 | −0.0515336 | 0.0515336 |
| 1738 | 13 | 48668773 | 48668909 | 0.05143636 | 0.051436362 |
| 1739 | 1 | 171455112 | 171455247 | 0.05141431 | 0.051414313 |
| 1740 | 20 | 62338520 | 62338943 | −0.0514131 | 0.0514131 |
| 1741 | 15 | 73343902 | 73344046 | 0.05135513 | 0.051355134 |
| 1742 | 12 | 115140502 | 115140651 | 0.05134348 | 0.051343483 |
| 1743 | 1 | 8938628 | 8938895 | 0.05134208 | 0.051342082 |
| 1744 | 19 | 44144229 | 44144418 | −0.0512635 | 0.0512635 |
| 1745 | 6 | 30658860 | 30659106 | −0.0512386 | 0.0512386 |
| 1746 | 17 | 55055059 | 55055191 | −0.0511311 | 0.0511311 |
| 1747 | 8 | 79578037 | 79578210 | 0.05112483 | 0.051124832 |
| 1748 | 22 | 19131929 | 19132204 | 0.05110088 | 0.051100876 |
| 1749 | 1 | 154914669 | 154914810 | −0.0510783 | 0.0510783 |
| 1750 | 2 | 70417900 | 70418035 | 0.05104214 | 0.05104214 |
| 1751 | 5 | 43484004 | 43484180 | −0.0509936 | 0.0509936 |
| 1752 | 1 | 111762443 | 111762731 | 0.05097278 | 0.05097278 |
| 1753 | 1 | 144932405 | 144932532 | −0.0507969 | 0.0507969 |
| 1754 | 15 | 52263709 | 52264282 | 0.05071807 | 0.05071807 |
| 1755 | 2 | 60784369 | 60784526 | 0.05068837 | 0.050688372 |
| 1756 | 11 | 73881841 | 73882211 | −0.050615 | 0.050615 |
| 1757 | 2 | 10443058 | 10443211 | 0.05050217 | 0.050502167 |
| 1758 | 11 | 73587650 | 73587800 | 0.05038989 | 0.050389888 |
| 1759 | 17 | 62502179 | 62502360 | −0.0503801 | 0.0503801 |
| 1760 | 7 | 10979693 | 10979839 | −0.0503678 | 0.0503678 |
| 1761 | 8 | 4849554 | 4849982 | −0.0502598 | 0.0502598 |
| 1762 | 13 | 100150911 | 100151151 | −0.0502581 | 0.0502581 |
| 1763 | 2 | 27886420 | 27886596 | −0.0501887 | 0.0501887 |
| 1764 | 2 | 98206096 | 98206246 | 0.05005404 | 0.050054045 |
| 1765 | 17 | 49197418 | 49197564 | −0.050052 | 0.050052 |
| 1766 | 3 | 100427964 | 100428237 | −0.0500271 | 0.0500271 |
| 1767 | 12 | 39299767 | 39299923 | −0.0500127 | 0.0500127 |
| 1768 | 15 | 89164433 | 89164685 | 0.05000653 | 0.050006525 |
| 1769 | 13 | 32421217 | 32421328 | 0.04993863 | 0.049938628 |
| 1770 | 19 | 40910012 | 40910152 | 0.04986422 | 0.049864221 |
| 1771 | 17 | 21414888 | 21415032 | −0.0498383 | 0.0498383 |
| 1772 | 7 | 75115498 | 75115764 | −0.0498005 | 0.0498005 |
| 1773 | 13 | 99960124 | 99960273 | 0.04969758 | 0.049697582 |
| 1774 | 3 | 53915721 | 53915863 | −0.0496109 | 0.0496109 |
| 1775 | 2 | 84686504 | 84686642 | −0.0496 | 0.0496 |
| 1776 | 20 | 61200507 | 61200653 | −0.0495834 | 0.0495834 |
| 1777 | 19 | 19517941 | 19518191 | 0.04958068 | 0.04958068 |
| 1778 | 1 | 180200031 | 180200170 | −0.0495544 | 0.0495544 |
| 1779 | 11 | 67120888 | 67121002 | 0.04951046 | 0.049510461 |
| 1780 | 20 | 35203463 | 35203707 | −0.0494617 | 0.0494617 |
| 1781 | 19 | 13858795 | 13858932 | 0.04937396 | 0.049373964 |
| 1782 | 12 | 122250073 | 122250213 | −0.0493274 | 0.0493274 |
| 1783 | 12 | 62997214 | 62997493 | 0.04932713 | 0.049327125 |
| 1784 | 1 | 155043484 | 155043623 | 0.04924142 | 0.049241425 |
| 1785 | 11 | 65657800 | 65657971 | 0.04921098 | 0.049210982 |
| 1786 | 17 | 79819259 | 79819400 | 0.049192 | 0.049191997 |
| 1787 | X | 39715004 | 39715151 | 0.04917484 | 0.04917484 |
| 1788 | 13 | 77566198 | 77566348 | −0.0491643 | 0.0491643 |
| 1789 | 15 | 73089156 | 73089404 | −0.0491582 | 0.0491582 |
| 1790 | 3 | 113250981 | 113251251 | −0.0491244 | 0.0491244 |
| 1791 | 20 | 25371601 | 25371889 | −0.0491035 | 0.0491035 |
| 1792 | 1 | 210506264 | 210506480 | −0.0490717 | 0.0490717 |
| 1793 | 13 | 41837586 | 41837715 | −0.0490299 | 0.0490299 |
| 1794 | 2 | 237077073 | 237077223 | −0.0490128 | 0.0490128 |
| 1795 | 6 | 159064940 | 159065129 | −0.0489476 | 0.0489476 |
| 1796 | 10 | 43571987 | 43572128 | −0.0489459 | 0.0489459 |
| 1797 | 10 | 133999444 | 133999779 | −0.0489307 | 0.0489307 |
| 1798 | 12 | 79257416 | 79257554 | 0.04889707 | 0.048897072 |
| 1799 | 5 | 132362007 | 132362255 | −0.048853 | 0.048853 |
| 1800 | 20 | 1783887 | 1784026 | 0.0488174 | 0.048817395 |
| 1801 | 12 | 31782787 | 31782939 | 0.04879046 | 0.048790458 |
| 1802 | 19 | 53193835 | 53193981 | −0.0487756 | 0.0487756 |
| 1803 | 15 | 40886098 | 40886310 | 0.04875791 | 0.048757909 |
| 1804 | 19 | 18942738 | 18942885 | −0.0487412 | 0.0487412 |
| 1805 | 7 | 27224233 | 27224412 | 0.04864668 | 0.048646675 |
| 1806 | 2 | 129075408 | 129075545 | 0.04859632 | 0.048596319 |
| 1807 | 19 | 47354246 | 47354359 | −0.0485737 | 0.0485737 |
| 1808 | 1 | 242011820 | 242011968 | −0.0485597 | 0.0485597 |
| 1809 | 4 | 11430371 | 11430520 | −0.0485307 | 0.0485307 |
| 1810 | 17 | 35849368 | 35849508 | 0.0483894 | 0.048389404 |
| 1811 | 10 | 111970870 | 111970970 | 0.04834725 | 0.048347254 |
| 1812 | 9 | 131103130 | 131103272 | 0.0483378 | 0.048337795 |
| 1813 | 15 | 66679727 | 66679869 | 0.04833618 | 0.048336178 |
| 1814 | 10 | 115934333 | 115934528 | 0.04832822 | 0.048328223 |
| 1815 | 14 | 23771982 | 23772271 | −0.0483223 | 0.0483223 |
| 1816 | 19 | 14200449 | 14200600 | 0.04826997 | 0.048269967 |
| 1817 | 5 | 42424764 | 42424898 | −0.0482561 | 0.0482561 |
| 1818 | 8 | 38854251 | 38854500 | 0.04825235 | 0.048252346 |
| 1819 | 2 | 87089294 | 87089498 | 0.0482215 | 0.048221499 |
| 1820 | 17 | 43298695 | 43298832 | −0.0481856 | 0.0481856 |
| 1821 | 10 | 54574351 | 54574498 | 0.0481273 | 0.048127297 |
| 1822 | 19 | 38810323 | 38810518 | 0.04806523 | 0.048065231 |
| 1823 | 7 | 121784026 | 121784181 | 0.04798682 | 0.047986822 |
| 1824 | 1 | 151966028 | 151966276 | −0.0479809 | 0.0479809 |
| 1825 | 16 | 30007320 | 30007672 | 0.04797944 | 0.047979439 |
| 1826 | 20 | 47835753 | 47836108 | 0.04791214 | 0.047912144 |
| 1827 | 1 | 1290647 | 1291088 | −0.0478627 | 0.0478627 |
| 1828 | 12 | 83080602 | 83080753 | −0.0478416 | 0.0478416 |
| 1829 | 22 | 39745675 | 39745822 | −0.0477305 | 0.0477305 |
| 1830 | 10 | 93805510 | 93805752 | 0.04772027 | 0.047720272 |
| 1831 | 3 | 186284957 | 186285106 | −0.0476875 | 0.0476875 |
| 1832 | 7 | 79763809 | 79763944 | −0.047639 | 0.047639 |
| 1833 | 16 | 71929385 | 71929591 | 0.04755952 | 0.047559522 |
| 1834 | 2 | 677296 | 677450 | 0.0474667 | 0.0474667 |
| 1835 | 2 | 242212393 | 242212533 | 0.04742846 | 0.047428456 |
| 1836 | 11 | 47600202 | 47600347 | −0.0473353 | 0.0473353 |
| 1837 | 5 | 72794002 | 72794147 | 0.04732577 | 0.047325774 |
| 1838 | 7 | 6048585 | 6049042 | 0.04725787 | 0.047257869 |
| 1839 | 19 | 40476841 | 40476988 | −0.0472511 | 0.0472511 |
| 1840 | 19 | 58838622 | 58838781 | 0.04723744 | 0.047237445 |
| 1841 | 17 | 7975743 | 7975956 | −0.0472168 | 0.0472168 |
| 1842 | 14 | 23388758 | 23388925 | 0.04713336 | 0.047133358 |
| 1843 | 11 | 46370564 | 46370934 | −0.0470419 | 0.0470419 |
| 1844 | 6 | 79576992 | 79577153 | 0.04698287 | 0.046982872 |
| 1845 | 13 | 112709731 | 112710596 | −0.0468739 | 0.0468739 |
| 1846 | 22 | 51066719 | 51066995 | 0.04683025 | 0.046830248 |
| 1847 | 4 | 144434335 | 144434651 | 0.04678915 | 0.046789154 |
| 1848 | 11 | 65640955 | 65641182 | 0.04678276 | 0.046782761 |
| 1849 | 8 | 123793519 | 123793664 | 0.04676172 | 0.046761719 |
| 1850 | 1 | 218520398 | 218520533 | −0.0467264 | 0.0467264 |
| 1851 | 22 | 22222245 | 22222386 | 0.04665172 | 0.04665172 |
| 1852 | 11 | 115530510 | 115530652 | 0.04657163 | 0.046571626 |
| 1853 | 16 | 67208492 | 67208785 | 0.04655716 | 0.046557158 |
| 1854 | 12 | 12764719 | 12764989 | 0.04653856 | 0.046538562 |
| 1855 | 7 | 20824859 | 20824992 | −0.0464874 | 0.0464874 |
| 1856 | 1 | 27320575 | 27320725 | −0.0464014 | 0.0464014 |
| 1857 | 11 | 636878 | 637266 | −0.0463005 | 0.0463005 |
| 1858 | 4 | 83145067 | 83145256 | 0.0462963 | 0.046296303 |
| 1859 | 5 | 36242375 | 36242522 | 0.04628304 | 0.046283042 |
| 1860 | 8 | 77912489 | 77912750 | −0.0462357 | 0.0462357 |
| 1861 | 22 | 46933015 | 46933151 | 0.0462179 | 0.046217902 |
| 1862 | 16 | 21312199 | 21312354 | 0.04618933 | 0.046189334 |
| 1863 | 19 | 4636385 | 4636522 | −0.0461608 | 0.0461608 |
| 1864 | 8 | 143591740 | 143591848 | 0.04615318 | 0.046153177 |
| 1865 | 19 | 58011512 | 58011630 | −0.0461135 | 0.0461135 |
| 1866 | 2 | 131862987 | 131863131 | −0.046045 | 0.046045 |
| 1867 | 2 | 71175640 | 71175798 | 0.04577325 | 0.045773253 |
| 1868 | 12 | 63544351 | 63544500 | 0.04573534 | 0.045735337 |
| 1869 | 8 | 22409494 | 22409649 | 0.045654 | 0.045653998 |
| 1870 | 2 | 191745390 | 191745820 | 0.04563672 | 0.045636721 |
| 1871 | 15 | 63486084 | 63486222 | 0.04562844 | 0.045628435 |
| 1872 | 17 | 37183314 | 37183795 | −0.0455938 | 0.0455938 |
| 1873 | 7 | 72972130 | 72972297 | −0.0455848 | 0.0455848 |
| 1874 | 1 | 81358242 | 81358510 | −0.045584 | 0.045584 |
| 1875 | 17 | 7388374 | 7388510 | 0.04554102 | 0.045541019 |
| 1876 | 8 | 39976586 | 39976950 | −0.0455398 | 0.0455398 |
| 1877 | 9 | 95055694 | 95055918 | −0.0455398 | 0.0455398 |
| 1878 | 20 | 30434458 | 30434605 | −0.0455101 | 0.0455101 |
| 1879 | 11 | 88071119 | 88071257 | 0.04550591 | 0.04550591 |
| 1880 | 16 | 68482511 | 68482663 | 0.04550231 | 0.045502306 |
| 1881 | 7 | 156803498 | 156803658 | −0.0454937 | 0.0454937 |
| 1882 | 22 | 38500823 | 38500950 | 0.04540493 | 0.045404932 |
| 1883 | 2 | 135016193 | 135016335 | −0.0453644 | 0.0453644 |
| 1884 | 17 | 8151150 | 8151319 | 0.0453434 | 0.045343404 |
| 1885 | 2 | 220306999 | 220307131 | −0.0453352 | 0.0453352 |
| 1886 | 12 | 93964615 | 93964874 | 0.04530025 | 0.045300254 |
| 1887 | 19 | 2900628 | 2900951 | −0.0452822 | 0.0452822 |
| 1888 | 13 | 41345400 | 41345723 | −0.045177 | 0.045177 |
| 1889 | 17 | 21179152 | 21179296 | −0.0451707 | 0.0451707 |
| 1890 | 5 | 34007811 | 34008030 | 0.04515938 | 0.04515938 |
| 1891 | 1 | 224545028 | 224545172 | 0.04508043 | 0.045080428 |
| 1892 | 8 | 87520823 | 87520966 | −0.0449377 | 0.0449377 |
| 1893 | 20 | 7999943 | 8000087 | −0.0448852 | 0.0448852 |
| 1894 | 2 | 32581634 | 32581793 | 0.0448844 | 0.044884402 |
| 1895 | 5 | 180480909 | 180481050 | −0.0448374 | 0.0448374 |
| 1896 | 12 | 56521649 | 56521776 | −0.0448289 | 0.0448289 |
| 1897 | 3 | 49313952 | 49314497 | 0.04479749 | 0.04479749 |
| 1898 | 16 | 30418644 | 30418782 | −0.0447669 | 0.0447669 |
| 1899 | 16 | 87635724 | 87635856 | −0.0447352 | 0.0447352 |
| 1900 | 2 | 208394381 | 208394586 | 0.04471218 | 0.044712181 |
| 1901 | 1 | 234613931 | 234614070 | 0.04470718 | 0.044707183 |
| 1902 | X | 132548111 | 132548253 | 0.04468863 | 0.044688628 |
| 1903 | 2 | 69533887 | 69534033 | 0.04461014 | 0.044610144 |
| 1904 | 2 | 64977211 | 64977368 | 0.04460487 | 0.044604868 |
| 1905 | 1 | 37939845 | 37939998 | −0.0445966 | 0.0445966 |
| 1906 | 9 | 99176003 | 99176154 | −0.0445749 | 0.0445749 |
| 1907 | 12 | 6472617 | 6472751 | −0.0445109 | 0.0445109 |
| 1908 | 11 | 64781407 | 64781513 | −0.0443823 | 0.0443823 |
| 1909 | 11 | 124543335 | 124543474 | 0.04437774 | 0.044377742 |
| 1910 | 11 | 134201875 | 134202019 | 0.04436293 | 0.044362928 |
| 1911 | 1 | 23857452 | 23857627 | −0.0443581 | 0.0443581 |
| 1912 | 1 | 243277530 | 243277771 | −0.0442492 | 0.0442492 |
| 1913 | 1 | 6520276 | 6520442 | 0.04415191 | 0.044151915 |
| 1914 | 7 | 127228760 | 127228913 | 0.04412369 | 0.044123687 |
| 1915 | 20 | 35401825 | 35401971 | −0.0440681 | 0.0440681 |
| 1916 | 11 | 67159701 | 67159855 | −0.0440434 | 0.0440434 |
| 1917 | 3 | 53925226 | 53925361 | −0.0440292 | 0.0440292 |
| 1918 | 6 | 25041895 | 25042259 | −0.0439948 | 0.0439948 |
| 1919 | 12 | 56842936 | 56843212 | 0.04387408 | 0.043874081 |
| 1920 | 9 | 132890688 | 132891509 | 0.04384845 | 0.043848454 |
| 1921 | 1 | 38512752 | 38512905 | −0.0438131 | 0.0438131 |
| 1922 | 12 | 14133205 | 14133361 | −0.0438116 | 0.0438116 |
| 1923 | 1 | 50882826 | 50883111 | −0.0438083 | 0.0438083 |
| 1924 | 7 | 91570424 | 91570576 | 0.04380213 | 0.043802126 |
| 1925 | 1 | 36689322 | 36689460 | −0.0437586 | 0.0437586 |
| 1926 | 20 | 55964826 | 55964884 | 0.04363551 | 0.043635506 |
| 1927 | 5 | 142783765 | 142783903 | −0.0435549 | 0.0435549 |
| 1928 | 2 | 42588262 | 42588404 | −0.0435116 | 0.0435116 |
| 1929 | 12 | 108954455 | 108954601 | −0.0434222 | 0.0434222 |
| 1930 | 7 | 2684815 | 2685354 | −0.0434102 | 0.0434102 |
| 1931 | 1 | 155145705 | 155145977 | 0.04339614 | 0.043396141 |
| 1932 | X | 44105096 | 44105245 | 0.04338967 | 0.043389667 |
| 1933 | 11 | 116643480 | 116643586 | 0.04336021 | 0.043360206 |
| 1934 | 6 | 28863316 | 28863601 | 0.04334023 | 0.043340225 |
| 1935 | 22 | 42343028 | 42343164 | 0.04331336 | 0.043313363 |
| 1936 | 19 | 54960386 | 54960520 | −0.0432931 | 0.0432931 |
| 1937 | 6 | 150262683 | 150262861 | 0.04326991 | 0.043269911 |
| 1938 | 10 | 70939838 | 70940074 | 0.04325666 | 0.043256658 |
| 1939 | 15 | 27018691 | 27018829 | −0.0432285 | 0.0432285 |
| 1940 | 11 | 121288002 | 121288177 | −0.0431727 | 0.0431727 |
| 1941 | 17 | 54991516 | 54991671 | 0.04313678 | 0.043136785 |
| 1942 | 5 | 14581455 | 14581600 | −0.0431136 | 0.0431136 |
| 1943 | 1 | 222886948 | 222887102 | 0.04309868 | 0.043098683 |
| 1944 | 1 | 249167412 | 249167744 | 0.04298873 | 0.042988727 |
| 1945 | 19 | 49956776 | 49956923 | −0.0429559 | 0.0429559 |
| 1946 | 2 | 45528864 | 45529069 | 0.04292999 | 0.042929986 |
| 1947 | 13 | 95363734 | 95363886 | −0.0429161 | 0.0429161 |
| 1948 | 3 | 50607055 | 50607205 | 0.04279731 | 0.042797314 |
| 1949 | 5 | 139047798 | 139048327 | −0.042782 | 0.042782 |
| 1950 | 19 | 1651460 | 1651602 | 0.04278151 | 0.042781507 |
| 1951 | 17 | 37607329 | 37607703 | 0.04274428 | 0.042744276 |
| 1952 | 1 | 174129061 | 174129199 | 0.04269479 | 0.042694791 |
| 1953 | 1 | 110880483 | 110880633 | 0.04269324 | 0.042693244 |
| 1954 | 19 | 17501897 | 17502006 | −0.0426278 | 0.0426278 |
| 1955 | 14 | 100071357 | 100071530 | 0.04256447 | 0.042564471 |
| 1956 | 11 | 66313749 | 66313904 | 0.04244152 | 0.042441524 |
| 1957 | 8 | 124170625 | 124170760 | −0.0424362 | 0.0424362 |
| 1958 | 17 | 42061007 | 42061152 | 0.04243087 | 0.042430873 |
| 1959 | 6 | 106433908 | 106434190 | −0.0423953 | 0.0423953 |
| 1960 | 17 | 48005579 | 48005779 | 0.04237416 | 0.04237416 |
| 1961 | 1 | 26856495 | 26856733 | −0.0423553 | 0.0423553 |
| 1962 | 1 | 44440131 | 44440314 | −0.0422898 | 0.0422898 |
| 1963 | 18 | 2972198 | 2972413 | −0.0421955 | 0.0421955 |
| 1964 | 17 | 19976792 | 19977254 | −0.0421571 | 0.0421571 |
| 1965 | 1 | 1623776 | 1623893 | 0.04213196 | 0.042131958 |
| 1966 | 14 | 92505997 | 92506209 | −0.0421084 | 0.0421084 |
| 1967 | 10 | 38265408 | 38265802 | −0.0420908 | 0.0420908 |
| 1968 | X | 49022528 | 49023980 | 0.04196231 | 0.04196231 |
| 1969 | 19 | 50879453 | 50879721 | −0.041954 | 0.041954 |
| 1970 | 2 | 170590358 | 170590504 | −0.0419426 | 0.0419426 |
| 1971 | 19 | 11205719 | 11206074 | −0.0418832 | 0.0418832 |
| 1972 | 20 | 44519501 | 44519761 | 0.04185323 | 0.041853226 |
| 1973 | 17 | 45908771 | 45909065 | 0.04175079 | 0.041750789 |
| 1974 | 17 | 46178145 | 46178416 | 0.04174811 | 0.041748108 |
| 1975 | 10 | 115743483 | 115743622 | 0.04171432 | 0.041714323 |
| 1976 | 12 | 25538216 | 25538357 | −0.0416855 | 0.0416855 |
| 1977 | X | 129087249 | 129087451 | −0.0416244 | 0.0416244 |
| 1978 | 18 | 24129600 | 24129746 | 0.04162406 | 0.041624055 |
| 1979 | 3 | 49507106 | 49507251 | 0.04161288 | 0.041612876 |
| 1980 | 9 | 124262264 | 124262424 | 0.04160082 | 0.041600819 |
| 1981 | 20 | 56285606 | 56285788 | 0.04159358 | 0.04159358 |
| 1982 | 16 | 2073240 | 2073356 | −0.0415733 | 0.0415733 |
| 1983 | 17 | 73936835 | 73936983 | 0.04154841 | 0.041548415 |
| 1984 | 12 | 54426650 | 54426784 | 0.04144209 | 0.041442091 |
| 1985 | 5 | 131629831 | 131630047 | 0.04139759 | 0.04139759 |
| 1986 | 3 | 52931777 | 52931909 | −0.0413969 | 0.0413969 |
| 1987 | 3 | 136581644 | 136581803 | 0.04136697 | 0.041366966 |
| 1988 | 7 | 99156283 | 99156459 | 0.04125517 | 0.041255167 |
| 1989 | 7 | 90225685 | 90225860 | −0.0411938 | 0.0411938 |
| 1990 | 10 | 119134633 | 119134786 | −0.0411024 | 0.0411024 |
| 1991 | 6 | 27356280 | 27356425 | 0.04104785 | 0.04104785 |
| 1992 | 10 | 80827159 | 80827384 | −0.0410083 | 0.0410083 |
| 1993 | 14 | 21979503 | 21979657 | −0.04099 | 0.04099 |
| 1994 | 13 | 96296913 | 96297082 | −0.040969 | 0.040969 |
| 1995 | 19 | 2478924 | 2479077 | −0.0409635 | 0.0409635 |
| 1996 | 10 | 95462621 | 95462777 | 0.04096084 | 0.040960836 |
| 1997 | 15 | 89907676 | 89907988 | −0.0409356 | 0.0409356 |
| 1998 | 19 | 42498582 | 42498673 | 0.04091679 | 0.040916788 |
| 1999 | 19 | 13227210 | 13227345 | −0.0408955 | 0.0408955 |
| 2000 | 17 | 79885581 | 79885902 | −0.0408866 | 0.0408866 |
| 2001 | 3 | 172310069 | 172310462 | −0.0408355 | 0.0408355 |
| 2002 | 7 | 72936594 | 72936764 | −0.0407666 | 0.0407666 |
| 2003 | 9 | 119449511 | 119449636 | −0.0407474 | 0.0407474 |
| 2004 | 20 | 3778801 | 3778951 | 0.0406502 | 0.0406502 |
| 2005 | 15 | 36871486 | 36871659 | −0.040642 | 0.040642 |
| 2006 | 20 | 35917994 | 35918301 | 0.04060326 | 0.040603259 |
| 2007 | 1 | 143766064 | 143766149 | −0.0405038 | 0.0405038 |
| 2008 | 17 | 44896356 | 44896494 | 0.04048394 | 0.04048394 |
| 2009 | 3 | 128274390 | 128274542 | 0.04038005 | 0.04038005 |
| 2010 | 1 | 61547421 | 61547561 | 0.04037367 | 0.040373667 |
| 2011 | 6 | 143141028 | 143141174 | 0.04031518 | 0.040315175 |
| 2012 | 1 | 236558795 | 236558939 | −0.0403135 | 0.0403135 |
| 2013 | 3 | 69255619 | 69255767 | 0.0401418 | 0.040141801 |
| 2014 | 1 | 84971047 | 84971192 | 0.04008118 | 0.040081178 |
| 2015 | 16 | 71928816 | 71929152 | 0.04001912 | 0.040019116 |
| 2016 | 3 | 27410953 | 27411129 | −0.0399777 | 0.0399777 |
| 2017 | 15 | 86234935 | 86235136 | −0.0398458 | 0.0398458 |
| 2018 | 19 | 49661029 | 49661142 | −0.0398375 | 0.0398375 |
| 2019 | 1 | 36396218 | 36396529 | −0.0398338 | 0.0398338 |
| 2020 | 10 | 73150285 | 73150670 | 0.03981124 | 0.039811244 |
| 2021 | 1 | 111217328 | 111217765 | 0.03978389 | 0.039783891 |
| 2022 | 8 | 818989 | 819379 | −0.0397645 | 0.0397645 |
| 2023 | 5 | 170744786 | 170745029 | −0.0397608 | 0.0397608 |
| 2024 | 6 | 149082248 | 149082395 | 0.03973148 | 0.039731482 |
| 2025 | 17 | 4384628 | 4384951 | −0.0396984 | 0.0396984 |
| 2026 | 1 | 115212822 | 115213002 | −0.0396878 | 0.0396878 |
| 2027 | 1 | 32135778 | 32136197 | −0.0396591 | 0.0396591 |
| 2028 | 3 | 186648470 | 186648611 | −0.0396435 | 0.0396435 |
| 2029 | 14 | 81397255 | 81397646 | −0.0396355 | 0.0396355 |
| 2030 | 6 | 28048881 | 28049054 | 0.03961381 | 0.039613812 |
| 2031 | 6 | 49518270 | 49518439 | 0.03961337 | 0.039613371 |
| 2032 | 8 | 146176190 | 146176334 | −0.0395699 | 0.0395699 |
| 2033 | 15 | 96906680 | 96907249 | −0.0395664 | 0.0395664 |
| 2034 | 3 | 101548036 | 101548220 | −0.039523 | 0.039523 |
| 2035 | 1 | 57045149 | 57045298 | 0.03951138 | 0.03951138 |
| 2036 | 2 | 203103354 | 203103500 | 0.03943677 | 0.039436772 |
| 2037 | 1 | 110753637 | 110753780 | −0.0393548 | 0.0393548 |
| 2038 | 5 | 41925044 | 41925288 | 0.03931507 | 0.039315074 |
| 2039 | 1 | 110162171 | 110162318 | 0.03929389 | 0.039293888 |
| 2040 | 20 | 40247156 | 40247271 | −0.0392637 | 0.0392637 |
| 2041 | X | 13018783 | 13018912 | 0.03925248 | 0.039252483 |
| 2042 | 18 | 43267263 | 43267438 | 0.03923433 | 0.03923433 |
| 2043 | 4 | 38655982 | 38656151 | 0.03922127 | 0.039221272 |
| 2044 | 14 | 89290880 | 89291028 | 0.03916265 | 0.039162648 |
| 2045 | 13 | 29394444 | 29394589 | 0.03910451 | 0.03910451 |
| 2046 | 16 | 69345607 | 69345753 | −0.0390459 | 0.0390459 |
| 2047 | 6 | 97731016 | 97731195 | 0.03902688 | 0.039026881 |
| 2048 | 15 | 91415045 | 91415182 | −0.0389943 | 0.0389943 |
| 2049 | 18 | 55470321 | 55470466 | −0.0388906 | 0.0388906 |
| 2050 | 11 | 87665519 | 87665654 | −0.0388898 | 0.0388898 |
| 2051 | 11 | 77185331 | 77185479 | −0.0388819 | 0.0388819 |
| 2052 | 14 | 91977161 | 91977335 | −0.0387815 | 0.0387815 |
| 2053 | 5 | 180076826 | 180077064 | 0.03874823 | 0.038748229 |
| 2054 | 10 | 71108768 | 71109003 | −0.0387333 | 0.0387333 |
| 2055 | 11 | 34075093 | 34075255 | 0.03872725 | 0.038727251 |
| 2056 | 19 | 50979667 | 50979837 | 0.03870553 | 0.038705529 |
| 2057 | 10 | 75385063 | 75385214 | 0.03869115 | 0.038691146 |
| 2058 | 1 | 38495817 | 38496143 | 0.03867319 | 0.038673187 |
| 2059 | 14 | 23563742 | 23563899 | 0.03865142 | 0.038651424 |
| 2060 | 17 | 7475736 | 7475997 | −0.0386313 | 0.0386313 |
| 2061 | 8 | 70984482 | 70984636 | −0.0385538 | 0.0385538 |
| 2062 | 16 | 4784281 | 4784422 | 0.03854492 | 0.038544919 |
| 2063 | 3 | 2139611 | 2139758 | 0.03853124 | 0.038531241 |
| 2064 | 3 | 111393261 | 111393668 | 0.03852736 | 0.03852736 |
| 2065 | 19 | 47852351 | 47852443 | 0.03850239 | 0.03850239 |
| 2066 | X | 103515494 | 103515658 | 0.03846291 | 0.038462906 |
| 2067 | 9 | 136023520 | 136023649 | 0.03845857 | 0.038458573 |
| 2068 | 14 | 81902277 | 81902418 | −0.0384486 | 0.0384486 |
| 2069 | 15 | 45879487 | 45879630 | 0.03838508 | 0.038385079 |
| 2070 | 12 | 109711221 | 109711356 | 0.03830688 | 0.038306882 |
| 2071 | 2 | 220094090 | 220094711 | 0.0383061 | 0.038306102 |
| 2072 | 22 | 31477566 | 31477705 | −0.0383051 | 0.0383051 |
| 2073 | 8 | 33370558 | 33370783 | −0.0383011 | 0.0383011 |
| 2074 | 11 | 62358924 | 62359206 | 0.03829562 | 0.03829562 |
| 2075 | 2 | 48009652 | 48009801 | −0.0382584 | 0.0382584 |
| 2076 | 6 | 113442005 | 113442150 | −0.0381858 | 0.0381858 |
| 2077 | 5 | 53813314 | 53813633 | −0.038107 | 0.038107 |
| 2078 | 2 | 39346807 | 39346947 | −0.0380955 | 0.0380955 |
| 2079 | 5 | 55117797 | 55117950 | −0.03804 | 0.03804 |
| 2080 | 19 | 12405351 | 12405491 | −0.0380335 | 0.0380335 |
| 2081 | 11 | 59383004 | 59383266 | −0.0380254 | 0.0380254 |
| 2082 | 13 | 42534898 | 42535059 | 0.0380013 | 0.038001303 |
| 2083 | 8 | 125576588 | 125576827 | −0.0379702 | 0.0379702 |
| 2084 | 19 | 35521133 | 35521228 | 0.03796463 | 0.037964631 |
| 2085 | 1 | 185014988 | 185015143 | −0.0379527 | 0.0379527 |
| 2086 | 19 | 15575605 | 15575754 | −0.0379027 | 0.0379027 |
| 2087 | 15 | 75199228 | 75199366 | 0.03784054 | 0.037840543 |
| 2088 | 10 | 105036623 | 105036928 | −0.0378223 | 0.0378223 |
| 2089 | 20 | 25677213 | 25677356 | −0.0377532 | 0.0377532 |
| 2090 | 8 | 80679234 | 80679373 | −0.0377262 | 0.0377262 |
| 2091 | 12 | 56615950 | 56616091 | −0.0376647 | 0.0376647 |
| 2092 | 19 | 20748098 | 20748240 | −0.0376398 | 0.0376398 |
| 2093 | 5 | 180229674 | 180229807 | −0.037622 | 0.037622 |
| 2094 | 15 | 40337341 | 40337481 | 0.03758047 | 0.037580471 |
| 2095 | 10 | 23488631 | 23488772 | −0.0375306 | 0.0375306 |
| 2096 | 10 | 52499670 | 52499811 | 0.03746612 | 0.03746612 |
| 2097 | 6 | 31831280 | 31831751 | 0.03745288 | 0.037452883 |
| 2098 | 1 | 46597559 | 46597699 | 0.03736545 | 0.037365455 |
| 2099 | 8 | 37963316 | 37963507 | −0.0373372 | 0.0373372 |
| 2100 | 6 | 91006521 | 91006733 | −0.0373208 | 0.0373208 |
| 2101 | 8 | 25242719 | 25242906 | 0.03727022 | 0.037270222 |
| 2102 | 6 | 112165026 | 112165161 | −0.037266 | 0.037266 |
| 2103 | 8 | 119963909 | 119964631 | −0.0372409 | 0.0372409 |
| 2104 | 18 | 33709364 | 33709511 | 0.0372392 | 0.037239197 |
| 2105 | 17 | 685023 | 685234 | 0.03718865 | 0.037188649 |
| 2106 | 3 | 99224907 | 99225050 | 0.03718531 | 0.03718531 |
| 2107 | 2 | 55458998 | 55459129 | 0.03717334 | 0.037173336 |
| 2108 | 11 | 108338172 | 108338411 | 0.03714952 | 0.037149521 |
| 2109 | 3 | 138658780 | 138659126 | −0.037138 | 0.037138 |
| 2110 | 6 | 10694730 | 10694969 | 0.03712628 | 0.037126275 |
| 2111 | 6 | 30646853 | 30647100 | −0.0371151 | 0.0371151 |
| 2112 | 2 | 121199879 | 121200232 | −0.0371114 | 0.0371114 |
| 2113 | 4 | 775538 | 775706 | −0.0371112 | 0.0371112 |
| 2114 | 5 | 174159345 | 174159657 | −0.037059 | 0.037059 |
| 2115 | 11 | 8289394 | 8289598 | −0.0370242 | 0.0370242 |
| 2116 | 2 | 132285934 | 132286039 | 0.03699717 | 0.036997173 |
| 2117 | 17 | 46619745 | 46619891 | −0.0369882 | 0.0369882 |
| 2118 | 6 | 131384549 | 131384745 | −0.0368607 | 0.0368607 |
| 2119 | 1 | 92950012 | 92950505 | 0.03682364 | 0.036823637 |
| 2120 | 7 | 32535373 | 32535518 | −0.036788 | 0.036788 |
| 2121 | 10 | 11674337 | 11674487 | −0.0367803 | 0.0367803 |
| 2122 | 1 | 151299726 | 151299859 | −0.0367802 | 0.0367802 |
| 2123 | 4 | 169738153 | 169738332 | −0.0367768 | 0.0367768 |
| 2124 | 10 | 104005414 | 104005555 | 0.03677667 | 0.03677667 |
| 2125 | 5 | 169064374 | 169064612 | 0.0367621 | 0.036762103 |
| 2126 | 15 | 81291986 | 81292279 | −0.0367212 | 0.0367212 |
| 2127 | 3 | 123969257 | 123969565 | 0.03653096 | 0.036530955 |
| 2128 | 12 | 109490555 | 109490705 | 0.03651835 | 0.036518348 |
| 2129 | 7 | 138145451 | 138145595 | −0.0365035 | 0.0365035 |
| 2130 | 17 | 38264570 | 38264731 | 0.03647527 | 0.036475265 |
| 2131 | 6 | 26250607 | 26250819 | 0.03639077 | 0.036390767 |
| 2132 | 19 | 8115286 | 8115537 | 0.0363628 | 0.036362803 |
| 2133 | 17 | 71640220 | 71640433 | 0.03633046 | 0.036330464 |
| 2134 | 15 | 90809072 | 90809232 | −0.0363247 | 0.0363247 |
| 2135 | 14 | 55878561 | 55878714 | 0.03628635 | 0.036286349 |
| 2136 | 9 | 102584411 | 102584565 | 0.03613224 | 0.036132241 |
| 2137 | 4 | 185655157 | 185655519 | −0.035992 | 0.035992 |
| 2138 | 3 | 18486161 | 18486297 | 0.03596883 | 0.035968832 |
| 2139 | 7 | 2883955 | 2884395 | −0.0359552 | 0.0359552 |
| 2140 | 16 | 23521543 | 23521675 | −0.0359189 | 0.0359189 |
| 2141 | 16 | 18573447 | 18573559 | 0.03587824 | 0.035878238 |
| 2142 | 14 | 90422265 | 90422415 | −0.0358766 | 0.0358766 |
| 2143 | 7 | 24612559 | 24612699 | 0.03586128 | 0.035861275 |
| 2144 | 1 | 53098561 | 53098726 | −0.0358515 | 0.0358515 |
| 2145 | 19 | 49653365 | 49653515 | 0.03584928 | 0.035849279 |
| 2146 | 16 | 86612372 | 86612521 | −0.0358423 | 0.0358423 |
| 2147 | 12 | 50794407 | 50794778 | 0.03578039 | 0.035780389 |
| 2148 | 8 | 53627548 | 53627692 | −0.0357573 | 0.0357573 |
| 2149 | 19 | 8641386 | 8641528 | −0.0357566 | 0.0357566 |
| 2150 | 4 | 87281210 | 87281350 | 0.03574537 | 0.035745373 |
| 2151 | 1 | 763052 | 763214 | 0.03573844 | 0.035738439 |
| 2152 | 11 | 122722415 | 122722554 | 0.03569335 | 0.03569335 |
| 2153 | 1 | 25175214 | 25175358 | −0.0356436 | 0.0356436 |
| 2154 | 11 | 85358926 | 85359100 | 0.0356192 | 0.035619197 |
| 2155 | 19 | 12250887 | 12251135 | 0.03557945 | 0.035579453 |
| 2156 | 16 | 15188156 | 15188404 | −0.0355464 | 0.0355464 |
| 2157 | 3 | 51975893 | 51976070 | −0.0355313 | 0.0355313 |
| 2158 | 15 | 67390688 | 67391064 | −0.0355077 | 0.0355077 |
| 2159 | 11 | 95523375 | 95523656 | −0.0355009 | 0.0355009 |
| 2160 | 4 | 56719776 | 56719994 | 0.03543461 | 0.035434612 |
| 2161 | 11 | 46141128 | 46141285 | −0.0354131 | 0.0354131 |
| 2162 | 19 | 2302607 | 2303130 | −0.035352 | 0.035352 |
| 2163 | 11 | 32112379 | 32112536 | 0.03531252 | 0.035312518 |
| 2164 | 19 | 44439531 | 44439674 | 0.0352813 | 0.035281298 |
| 2165 | 10 | 104221144 | 104221291 | −0.0352555 | 0.0352555 |
| 2166 | 21 | 46293989 | 46294130 | 0.03522284 | 0.035222841 |
| 2167 | 11 | 102217701 | 102217845 | 0.03511008 | 0.035110082 |
| 2168 | 18 | 39535018 | 39535254 | 0.03509875 | 0.035098746 |
| 2169 | 18 | 32924469 | 32924718 | 0.03498898 | 0.034988979 |
| 2170 | 3 | 33840389 | 33840529 | 0.03496423 | 0.034964231 |
| 2171 | 7 | 132937564 | 132937831 | 0.03493214 | 0.034932144 |
| 2172 | 1 | 56842354 | 56842515 | 0.0349305 | 0.034930501 |
| 2173 | 20 | 30467550 | 30467699 | 0.03492447 | 0.034924468 |
| 2174 | 4 | 24802292 | 24802490 | −0.0349083 | 0.0349083 |
| 2175 | 5 | 139127623 | 139127768 | −0.034894 | 0.034894 |
| 2176 | 15 | 43212286 | 43212429 | −0.034829 | 0.034829 |
| 2177 | 8 | 96281426 | 96281577 | 0.03478944 | 0.034789443 |
| 2178 | 4 | 106394742 | 106394895 | 0.0347797 | 0.034779695 |
| 2179 | 16 | 5003723 | 5003873 | 0.03476396 | 0.03476396 |
| 2180 | 12 | 112819917 | 112820073 | 0.03476203 | 0.034762034 |
| 2181 | 10 | 3462296 | 3462431 | −0.0347456 | 0.0347456 |
| 2182 | 11 | 536265 | 536411 | −0.0347297 | 0.0347297 |
| 2183 | 2 | 217497811 | 217497952 | 0.03467795 | 0.034677946 |
| 2184 | 1 | 117664395 | 117664678 | 0.03449293 | 0.034492928 |
| 2185 | 15 | 75248696 | 75248842 | 0.03446553 | 0.034465531 |
| 2186 | 1 | 15930047 | 15930288 | 0.03445118 | 0.03445118 |
| 2187 | 7 | 65670051 | 65670332 | −0.0344396 | 0.0344396 |
| 2188 | 12 | 125240106 | 125240252 | −0.0343775 | 0.0343775 |
| 2189 | 16 | 81272333 | 81272468 | −0.0343236 | 0.0343236 |
| 2190 | 7 | 64126874 | 64127030 | −0.0342962 | 0.0342962 |
| 2191 | 20 | 15876174 | 15876523 | −0.0342425 | 0.0342425 |
| 2192 | 16 | 23848360 | 23848507 | 0.03423355 | 0.034233549 |
| 2193 | 10 | 120354004 | 120354146 | −0.0341857 | 0.0341857 |
| 2194 | 20 | 57224582 | 57225357 | −0.0341583 | 0.0341583 |
| 2195 | X | 55035621 | 55035765 | 0.03413458 | 0.03413458 |
| 2196 | 14 | 68141581 | 68141863 | 0.03412424 | 0.034124242 |
| 2197 | 13 | 21141548 | 21141704 | 0.0341004 | 0.034100402 |
| 2198 | 2 | 24306993 | 24307134 | 0.03405893 | 0.034058935 |
| 2199 | 17 | 21030175 | 21030305 | −0.034048 | 0.034048 |
| 2200 | 17 | 40831941 | 40832076 | 0.03402854 | 0.034028535 |
| 2201 | 4 | 41371658 | 41371921 | −0.0340053 | 0.0340053 |
| 2202 | 18 | 28622835 | 28623019 | −0.0339883 | 0.0339883 |
| 2203 | 12 | 50560786 | 50560954 | −0.0339828 | 0.0339828 |
| 2204 | 10 | 122739047 | 122739201 | 0.03397208 | 0.03397208 |
| 2205 | 19 | 2739973 | 2740187 | 0.03384813 | 0.033848133 |
| 2206 | 1 | 43311952 | 43312199 | −0.0338142 | 0.0338142 |
| 2207 | 18 | 47814070 | 47814314 | 0.03380324 | 0.033803244 |
| 2208 | 8 | 9413311 | 9413547 | 0.03377305 | 0.033773049 |
| 2209 | 4 | 96469441 | 96469700 | 0.03374032 | 0.033740323 |
| 2210 | 3 | 49851166 | 49851314 | −0.0336811 | 0.0336811 |
| 2211 | 9 | 97623781 | 97624032 | −0.0336136 | 0.0336136 |
| 2212 | 7 | 99063428 | 99063577 | 0.03361237 | 0.033612371 |
| 2213 | 8 | 100025528 | 100025671 | −0.0334769 | 0.0334769 |
| 2214 | 19 | 39832481 | 39832607 | 0.03339299 | 0.033392992 |
| 2215 | 6 | 26183762 | 26183898 | −0.0333862 | 0.0333862 |
| 2216 | 7 | 42277270 | 42277487 | 0.03336488 | 0.03336488 |
| 2217 | 3 | 10029127 | 10029259 | 0.03334759 | 0.033347588 |
| 2218 | 9 | 98111370 | 98111592 | −0.0332668 | 0.0332668 |
| 2219 | 7 | 32338871 | 32339023 | 0.03322946 | 0.033229456 |
| 2220 | 3 | 193853842 | 193854003 | 0.0331916 | 0.0331916 |
| 2221 | 17 | 73734350 | 73734497 | −0.0331797 | 0.0331797 |
| 2222 | 5 | 392570 | 392906 | 0.03316752 | 0.033167524 |
| 2223 | 8 | 124780272 | 124780421 | 0.03316752 | 0.033167517 |
| 2224 | 7 | 151722394 | 151722532 | 0.03310852 | 0.033108519 |
| 2225 | 11 | 73018232 | 73018376 | 0.03310801 | 0.033108006 |
| 2226 | 17 | 37040212 | 37040418 | 0.03307995 | 0.033079947 |
| 2227 | 10 | 97416487 | 97416767 | 0.03302027 | 0.033020272 |
| 2228 | 13 | 47370964 | 47371125 | 0.03293716 | 0.032937164 |
| 2229 | 2 | 106361945 | 106362097 | −0.0329245 | 0.0329245 |
| 2230 | 9 | 135870001 | 135870216 | −0.0328774 | 0.0328774 |
| 2231 | 8 | 21917374 | 21917724 | −0.0328434 | 0.0328434 |
| 2232 | 14 | 57273215 | 57273356 | −0.0328292 | 0.0328292 |
| 2233 | 19 | 13842492 | 13842658 | −0.0328256 | 0.0328256 |
| 2234 | 3 | 50396604 | 50396747 | −0.0327005 | 0.0327005 |
| 2235 | 14 | 24616932 | 24617081 | 0.03266444 | 0.03266444 |
| 2236 | 3 | 16553437 | 16553929 | −0.0326386 | 0.0326386 |
| 2237 | 3 | 49157834 | 49157980 | −0.0326351 | 0.0326351 |
| 2238 | 5 | 72990128 | 72990298 | 0.03261689 | 0.032616894 |
| 2239 | 11 | 120664769 | 120664913 | −0.0326058 | 0.0326058 |
| 2240 | 9 | 131710371 | 131710594 | 0.03260463 | 0.032604634 |
| 2241 | 11 | 68847902 | 68848092 | −0.0325907 | 0.0325907 |
| 2242 | 2 | 9983779 | 9983918 | 0.03257018 | 0.032570179 |
| 2243 | 2 | 101869897 | 101870034 | −0.0325503 | 0.0325503 |
| 2244 | 2 | 219433552 | 219433689 | 0.03249014 | 0.032490136 |
| 2245 | 5 | 139167456 | 139167593 | 0.03235601 | 0.032356012 |
| 2246 | 19 | 571247 | 571495 | 0.03232752 | 0.032327517 |
| 2247 | X | 13770983 | 13771155 | 0.03231361 | 0.03231361 |
| 2248 | 1 | 33896840 | 33896982 | 0.03230511 | 0.032305109 |
| 2249 | 10 | 77168402 | 77168609 | 0.03224349 | 0.03224349 |
| 2250 | 17 | 37081795 | 37081947 | 0.03224123 | 0.032241226 |
| 2251 | 4 | 76555694 | 76555936 | 0.03223399 | 0.03223399 |
| 2252 | 9 | 101403823 | 101403973 | 0.03219256 | 0.032192555 |
| 2253 | 15 | 70820748 | 70820906 | −0.0320974 | 0.0320974 |
| 2254 | 15 | 45428694 | 45428855 | −0.0320964 | 0.0320964 |
| 2255 | 1 | 229163453 | 229163670 | −0.0320867 | 0.0320867 |
| 2256 | 5 | 169724195 | 169724342 | −0.0320787 | 0.0320787 |
| 2257 | 17 | 42092292 | 42092483 | 0.03202659 | 0.032026593 |
| 2258 | 2 | 44588945 | 44589232 | 0.03200138 | 0.03200138 |
| 2259 | 8 | 130951999 | 130952217 | 0.03189861 | 0.03189861 |
| 2260 | 13 | 102569125 | 102569577 | −0.031877 | 0.031877 |
| 2261 | 3 | 11760531 | 11760676 | −0.0318647 | 0.0318647 |
| 2262 | 22 | 42486673 | 42486755 | 0.03180229 | 0.031802287 |
| 2263 | 3 | 152878285 | 152878432 | 0.03178919 | 0.03178919 |
| 2264 | X | 83757356 | 83757570 | 0.03177149 | 0.031771486 |
| 2265 | 7 | 14030057 | 14030192 | 0.0317073 | 0.031707301 |
| 2266 | 19 | 3505874 | 3506018 | −0.0317069 | 0.0317069 |
| 2267 | 18 | 812417 | 812666 | −0.0316987 | 0.0316987 |
| 2268 | 11 | 126081924 | 126082070 | 0.03169228 | 0.031692283 |
| 2269 | 16 | 67906831 | 67907077 | 0.03166173 | 0.031661725 |
| 2270 | 8 | 72917038 | 72917587 | −0.0316456 | 0.0316456 |
| 2271 | 19 | 12405760 | 12405936 | −0.0316425 | 0.0316425 |
| 2272 | 19 | 41256315 | 41256407 | −0.0316206 | 0.0316206 |
| 2273 | 1 | 51435539 | 51435695 | 0.03158644 | 0.031586441 |
| 2274 | 16 | 475416 | 475560 | 0.03152746 | 0.03152746 |
| 2275 | 2 | 238864556 | 238865237 | −0.0315116 | 0.0315116 |
| 2276 | 19 | 662254 | 662392 | 0.03148049 | 0.031480492 |
| 2277 | 1 | 166944750 | 166944928 | −0.0314482 | 0.0314482 |
| 2278 | 5 | 118681638 | 118681774 | 0.0313988 | 0.031398802 |
| 2279 | 4 | 38308322 | 38308467 | 0.03139435 | 0.031394347 |
| 2280 | 10 | 32635741 | 32635878 | 0.03136709 | 0.031367086 |
| 2281 | 17 | 6939227 | 6939378 | −0.0313134 | 0.0313134 |
| 2282 | 7 | 139476839 | 139476985 | 0.03124404 | 0.031244043 |
| 2283 | 9 | 102669395 | 102669535 | 0.03121711 | 0.031217108 |
| 2284 | 6 | 31926490 | 31926982 | 0.03110805 | 0.031108054 |
| 2285 | 13 | 37393843 | 37393996 | −0.0310751 | 0.0310751 |
| 2286 | 2 | 66801800 | 66801947 | −0.0310031 | 0.0310031 |
| 2287 | 13 | 33000256 | 33000399 | −0.0309924 | 0.0309924 |
| 2288 | 2 | 10442136 | 10442386 | −0.03093 | 0.03093 |
| 2289 | X | 129219827 | 129219959 | 0.03091585 | 0.030915849 |
| 2290 | 4 | 186346979 | 186347188 | 0.03087745 | 0.030877446 |
| 2291 | 9 | 5841939 | 5842078 | −0.0308301 | 0.0308301 |
| 2292 | 5 | 141071823 | 141071959 | −0.0307828 | 0.0307828 |
| 2293 | 6 | 27094984 | 27095193 | 0.03078005 | 0.030780049 |
| 2294 | 11 | 105947184 | 105947475 | 0.03077907 | 0.030779075 |
| 2295 | 19 | 7587235 | 7587585 | 0.03076786 | 0.030767864 |
| 2296 | 11 | 2909646 | 2909790 | 0.03070609 | 0.030706086 |
| 2297 | 3 | 118959389 | 118959641 | −0.0306949 | 0.0306949 |
| 2298 | 1 | 119549234 | 119549388 | 0.03067518 | 0.030675175 |
| 2299 | 1 | 36863519 | 36863728 | 0.0306448 | 0.030644803 |
| 2300 | 7 | 72936535 | 72936573 | 0.03062206 | 0.030622058 |
| 2301 | 6 | 135484562 | 135484708 | 0.03053193 | 0.030531934 |
| 2302 | 1 | 71546408 | 71546546 | 0.03053079 | 0.030530789 |
| 2303 | 17 | 66098035 | 66098178 | −0.030512 | 0.030512 |
| 2304 | 8 | 77911599 | 77911748 | −0.030464 | 0.030464 |
| 2305 | 12 | 120426470 | 120426699 | −0.0304511 | 0.0304511 |
| 2306 | 3 | 87275913 | 87276053 | −0.0304167 | 0.0304167 |
| 2307 | 14 | 57271965 | 57272184 | −0.0303753 | 0.0303753 |
| 2308 | 19 | 54599141 | 54599364 | 0.03037244 | 0.030372445 |
| 2309 | 1 | 235490677 | 235490811 | −0.0303632 | 0.0303632 |
| 2310 | 2 | 209130966 | 209131062 | −0.0303052 | 0.0303052 |
| 2311 | 14 | 102785904 | 102786048 | −0.030278 | 0.030278 |
| 2312 | 17 | 36905918 | 36906082 | 0.03023802 | 0.030238021 |
| 2313 | 6 | 43485033 | 43485193 | −0.0302323 | 0.0302323 |
| 2314 | 2 | 27886679 | 27886823 | −0.0301233 | 0.0301233 |
| 2315 | 12 | 9800674 | 9800882 | −0.0301085 | 0.0301085 |
| 2316 | 2 | 230931713 | 230931990 | 0.0300612 | 0.030061195 |
| 2317 | 6 | 117198107 | 117198267 | 0.03005838 | 0.030058377 |
| 2318 | 7 | 99155688 | 99156205 | −0.0300334 | 0.0300334 |
| 2319 | 16 | 19179176 | 19179331 | 0.03003259 | 0.030032593 |
| 2320 | 13 | 45885815 | 45885953 | 0.03002332 | 0.030023324 |
| 2321 | 5 | 5494911 | 5495060 | −0.0300011 | 0.0300011 |
| 2322 | 14 | 55196891 | 55197206 | 0.02995606 | 0.029956062 |
| 2323 | 17 | 40688584 | 40688838 | 0.02994612 | 0.029946123 |
| 2324 | 6 | 26197657 | 26197810 | −0.029933 | 0.029933 |
| 2325 | 7 | 97501650 | 97501939 | 0.02990407 | 0.029904072 |
| 2326 | 5 | 1112032 | 1112385 | −0.0298834 | 0.0298834 |
| 2327 | 2 | 70418045 | 70418253 | 0.0298832 | 0.029883198 |
| 2328 | 15 | 51200186 | 51200446 | 0.02984212 | 0.02984212 |
| 2329 | 12 | 75905151 | 75905298 | 0.02978694 | 0.029786943 |
| 2330 | 10 | 61970065 | 61970308 | 0.02977618 | 0.029776183 |
| 2331 | 10 | 94050441 | 94050580 | 0.02974029 | 0.029740293 |
| 2332 | 6 | 27280344 | 27280486 | 0.02965293 | 0.029652927 |
| 2333 | 8 | 124169963 | 124170098 | −0.0296355 | 0.0296355 |
| 2334 | 12 | 118813863 | 118813996 | −0.0296274 | 0.0296274 |
| 2335 | 12 | 32113021 | 32113162 | −0.0296013 | 0.0296013 |
| 2336 | 8 | 17942364 | 17942606 | 0.02956297 | 0.029562975 |
| 2337 | 12 | 80328084 | 80328227 | 0.02949736 | 0.029497356 |
| 2338 | 2 | 71559261 | 71559401 | −0.029465 | 0.029465 |
| 2339 | 1 | 91301126 | 91301274 | 0.02945565 | 0.029455649 |
| 2340 | 6 | 35995554 | 35995764 | 0.02938214 | 0.029382138 |
| 2341 | 13 | 31618672 | 31618814 | −0.0293511 | 0.0293511 |
| 2342 | 14 | 24711525 | 24711707 | −0.0293449 | 0.0293449 |
| 2343 | 3 | 14988759 | 14988918 | 0.02934242 | 0.029342423 |
| 2344 | 9 | 6412911 | 6413048 | −0.0292593 | 0.0292593 |
| 2345 | 11 | 35684719 | 35684862 | −0.0292519 | 0.0292519 |
| 2346 | 17 | 18061489 | 18061636 | −0.0292503 | 0.0292503 |
| 2347 | 13 | 43169157 | 43169316 | −0.0292497 | 0.0292497 |
| 2348 | 1 | 27191184 | 27191334 | 0.02919978 | 0.029199777 |
| 2349 | 10 | 3807256 | 3807553 | 0.02917495 | 0.029174954 |
| 2350 | 5 | 89705799 | 89706079 | −0.0291422 | 0.0291422 |
| 2351 | 7 | 19157149 | 19157340 | 0.02912969 | 0.029129691 |
| 2352 | 19 | 47633860 | 47634173 | 0.02903297 | 0.02903297 |
| 2353 | 17 | 46671418 | 46671573 | 0.02903149 | 0.029031487 |
| 2354 | 19 | 57922240 | 57922398 | −0.0290116 | 0.0290116 |
| 2355 | 2 | 109647626 | 109647910 | −0.0290055 | 0.0290055 |
| 2356 | 4 | 44450807 | 44451159 | 0.02900067 | 0.029000672 |
| 2357 | 10 | 121484819 | 121484981 | 0.02899961 | 0.028999612 |
| 2358 | 17 | 72968918 | 72969117 | 0.02897104 | 0.028971043 |
| 2359 | 3 | 128968274 | 128968493 | 0.02895222 | 0.028952223 |
| 2360 | 1 | 21985908 | 21986060 | 0.02891198 | 0.028911976 |
| 2361 | 19 | 44617748 | 44617886 | 0.02889936 | 0.028899361 |
| 2362 | 11 | 66726154 | 66726307 | −0.0288818 | 0.0288818 |
| 2363 | 10 | 103815565 | 103815697 | 0.02881991 | 0.028819905 |
| 2364 | 13 | 112715334 | 112716239 | −0.0287746 | 0.0287746 |
| 2365 | 2 | 38770324 | 38770466 | 0.02871377 | 0.028713767 |
| 2366 | 17 | 4853679 | 4853985 | −0.0287078 | 0.0287078 |
| 2367 | 20 | 22562419 | 22564254 | −0.0286759 | 0.0286759 |
| 2368 | 18 | 57365048 | 57365175 | 0.02858607 | 0.02858607 |
| 2369 | 16 | 30886512 | 30886657 | −0.028548 | 0.028548 |
| 2370 | 1 | 155948074 | 155948264 | 0.028418 | 0.028418002 |
| 2371 | 12 | 6714865 | 6715005 | 0.02840456 | 0.028404562 |
| 2372 | 1 | 14507894 | 14508052 | 0.02834284 | 0.028342839 |
| 2373 | 21 | 43482122 | 43483179 | −0.0283405 | 0.0283405 |
| 2374 | 17 | 45266112 | 45266273 | 0.02833294 | 0.028332939 |
| 2375 | 11 | 68039622 | 68039766 | −0.0282739 | 0.0282739 |
| 2376 | 2 | 199009496 | 199009665 | −0.0282723 | 0.0282723 |
| 2377 | 6 | 43603353 | 43603503 | 0.02826496 | 0.028264964 |
| 2378 | 11 | 46403337 | 46403480 | −0.0282302 | 0.0282302 |
| 2379 | 16 | 11043239 | 11043425 | −0.0281513 | 0.0281513 |
| 2380 | 4 | 99849576 | 99849934 | −0.028144 | 0.028144 |
| 2381 | 8 | 8751249 | 8751387 | −0.0280899 | 0.0280899 |
| 2382 | 19 | 19249123 | 19249533 | −0.0280893 | 0.0280893 |
| 2383 | 1 | 35734726 | 35734867 | −0.0280578 | 0.0280578 |
| 2384 | 8 | 27491129 | 27491320 | 0.02805436 | 0.028054362 |
| 2385 | 9 | 117128714 | 117128905 | 0.02802503 | 0.028025031 |
| 2386 | 4 | 15006563 | 15006808 | −0.0280021 | 0.0280021 |
| 2387 | 11 | 66189220 | 66189358 | −0.0279905 | 0.0279905 |
| 2388 | 4 | 185394083 | 185394219 | 0.02797242 | 0.027972419 |
| 2389 | 2 | 97203695 | 97204830 | 0.02795296 | 0.027952958 |
| 2390 | 10 | 15138720 | 15138875 | 0.02792636 | 0.027926359 |
| 2391 | 20 | 45402414 | 45402551 | −0.0279238 | 0.0279238 |
| 2392 | 14 | 96829512 | 96829671 | 0.02791186 | 0.027911858 |
| 2393 | 15 | 45747038 | 45747306 | 0.02783855 | 0.027838546 |
| 2394 | 14 | 35591760 | 35591906 | 0.02781582 | 0.027815824 |
| 2395 | 22 | 39437270 | 39437501 | −0.0278157 | 0.0278157 |
| 2396 | 17 | 48171722 | 48171928 | −0.027802 | 0.027802 |
| 2397 | 9 | 95168959 | 95169113 | −0.0277917 | 0.0277917 |
| 2398 | 10 | 118928347 | 118928839 | 0.0277781 | 0.027778102 |
| 2399 | 2 | 45157259 | 45157793 | −0.0277534 | 0.0277534 |
| 2400 | 1 | 21023424 | 21023568 | 0.02774778 | 0.027747778 |
| 2401 | 4 | 2935616 | 2935768 | −0.0276916 | 0.0276916 |
| 2402 | 17 | 46970343 | 46970479 | −0.0276194 | 0.0276194 |
| 2403 | 1 | 200011604 | 200011770 | 0.02760893 | 0.027608927 |
| 2404 | 3 | 40547626 | 40547783 | −0.0275812 | 0.0275812 |
| 2405 | 5 | 159546139 | 159546280 | 0.02751246 | 0.027512456 |
| 2406 | 2 | 38607602 | 38607847 | 0.0274942 | 0.027494196 |
| 2407 | 10 | 28031191 | 28031350 | 0.02740709 | 0.027407091 |
| 2408 | 1 | 144593264 | 144593380 | −0.0273665 | 0.0273665 |
| 2409 | 1 | 155052116 | 155052269 | 0.02733614 | 0.027336139 |
| 2410 | 14 | 36003288 | 36003602 | 0.02729416 | 0.027294158 |
| 2411 | 1 | 236767476 | 236767692 | 0.02728061 | 0.027280611 |
| 2412 | 3 | 53880286 | 53880434 | −0.027199 | 0.027199 |
| 2413 | 3 | 141206825 | 141206978 | −0.027139 | 0.027139 |
| 2414 | 19 | 58912645 | 58912797 | −0.0270727 | 0.0270727 |
| 2415 | 16 | 46918084 | 46918228 | −0.0270495 | 0.0270495 |
| 2416 | 20 | 3082808 | 3083028 | −0.0269773 | 0.0269773 |
| 2417 | 15 | 65203835 | 65204183 | 0.02695964 | 0.026959644 |
| 2418 | 2 | 54014383 | 54014529 | 0.02693241 | 0.026932415 |
| 2419 | 8 | 99129702 | 99129846 | 0.02692789 | 0.026927887 |
| 2420 | 15 | 96888734 | 96889604 | −0.0269235 | 0.0269235 |
| 2421 | 4 | 39979705 | 39979854 | 0.02684662 | 0.026846616 |
| 2422 | 19 | 15529373 | 15529526 | 0.02680884 | 0.026808843 |
| 2423 | 19 | 42746805 | 42746981 | −0.0267783 | 0.0267783 |
| 2424 | 12 | 31477365 | 31477499 | 0.02676091 | 0.026760908 |
| 2425 | 15 | 75230202 | 75230351 | −0.0266936 | 0.0266936 |
| 2426 | 22 | 36847877 | 36848187 | −0.0266912 | 0.0266912 |
| 2427 | 13 | 23733782 | 23733925 | −0.0266346 | 0.0266346 |
| 2428 | 14 | 55034468 | 55034745 | −0.0265633 | 0.0265633 |
| 2429 | 15 | 43622177 | 43622323 | −0.0265505 | 0.0265505 |
| 2430 | 12 | 42876809 | 42876946 | −0.0264536 | 0.0264536 |
| 2431 | 7 | 156433367 | 156433596 | 0.02639649 | 0.026396495 |
| 2432 | 19 | 7994399 | 7994542 | −0.0263653 | 0.0263653 |
| 2433 | 3 | 38178189 | 38178332 | −0.0263111 | 0.0263111 |
| 2434 | 11 | 797964 | 798191 | −0.0262587 | 0.0262587 |
| 2435 | 1 | 95221295 | 95221474 | −0.026187 | 0.026187 |
| 2436 | 4 | 146018888 | 146019027 | 0.02617906 | 0.026179059 |
| 2437 | 13 | 99064079 | 99064490 | 0.02614283 | 0.026142831 |
| 2438 | X | 136115909 | 136116222 | −0.0261264 | 0.0261264 |
| 2439 | 7 | 142490883 | 142491018 | 0.02612457 | 0.026124572 |
| 2440 | 7 | 97601565 | 97601778 | 0.02612065 | 0.026120653 |
| 2441 | 7 | 134143835 | 134144183 | 0.02611415 | 0.026114145 |
| 2442 | 6 | 26156157 | 26156306 | −0.0260271 | 0.0260271 |
| 2443 | 3 | 38179800 | 38179971 | −0.0260235 | 0.0260235 |
| 2444 | 5 | 52776264 | 52776414 | −0.0260014 | 0.0260014 |
| 2445 | 2 | 241458869 | 241459036 | 0.02597598 | 0.025975977 |
| 2446 | 16 | 6948482 | 6948621 | 0.02596089 | 0.025960888 |
| 2447 | 22 | 30711089 | 30711247 | −0.0259586 | 0.0259586 |
| 2448 | 1 | 113933860 | 113934032 | 0.02592096 | 0.025920957 |
| 2449 | 21 | 40556037 | 40556181 | −0.0258965 | 0.0258965 |
| 2450 | 12 | 100594716 | 100594865 | 0.02588037 | 0.02588037 |
| 2451 | 2 | 27651492 | 27651628 | −0.0258633 | 0.0258633 |
| 2452 | 7 | 27779519 | 27779832 | 0.02582783 | 0.025827825 |
| 2453 | 11 | 130786447 | 130786700 | −0.025826 | 0.025826 |
| 2454 | 20 | 49411515 | 49411665 | −0.025826 | 0.025826 |
| 2455 | 22 | 19434844 | 19434991 | 0.02577756 | 0.02577756 |
| 2456 | 14 | 52118034 | 52118179 | −0.0257746 | 0.0257746 |
| 2457 | 11 | 46260718 | 46260860 | 0.02576182 | 0.025761817 |
| 2458 | 4 | 41748495 | 41748710 | −0.0257486 | 0.0257486 |
| 2459 | 7 | 23145191 | 23145394 | 0.02572936 | 0.02572936 |
| 2460 | 16 | 85722233 | 85722373 | −0.025725 | 0.025725 |
| 2461 | 17 | 54991324 | 54991426 | −0.025721 | 0.025721 |
| 2462 | 3 | 197477153 | 197477326 | −0.0257188 | 0.0257188 |
| 2463 | 3 | 53380731 | 53380869 | −0.0256786 | 0.0256786 |
| 2464 | 16 | 21371499 | 21371756 | 0.02565753 | 0.025657529 |
| 2465 | 6 | 52441643 | 52441794 | 0.02563354 | 0.025633539 |
| 2466 | 13 | 52585738 | 52585881 | 0.02562988 | 0.025629878 |
| 2467 | 9 | 102585241 | 102585383 | 0.02559556 | 0.02559556 |
| 2468 | 2 | 175643253 | 175643431 | −0.0255887 | 0.0255887 |
| 2469 | 11 | 20180362 | 20180501 | 0.02553036 | 0.025530358 |
| 2470 | 1 | 26633338 | 26633417 | 0.02547583 | 0.025475829 |
| 2471 | 17 | 42072292 | 42072447 | −0.025424 | 0.025424 |
| 2472 | 19 | 40023205 | 40023627 | −0.0253935 | 0.0253935 |
| 2473 | 3 | 47517183 | 47517322 | −0.025373 | 0.025373 |
| 2474 | 19 | 8009025 | 8009155 | 0.02529847 | 0.025298467 |
| 2475 | 7 | 29234021 | 29234159 | 0.02525877 | 0.025258766 |
| 2476 | 8 | 104033556 | 104033817 | −0.0252331 | 0.0252331 |
| 2477 | 17 | 38219758 | 38219919 | −0.0252304 | 0.0252304 |
| 2478 | 2 | 20424246 | 20424410 | 0.02522332 | 0.02522332 |
| 2479 | 2 | 173327793 | 173327943 | −0.0251899 | 0.0251899 |
| 2480 | 17 | 62493119 | 62493289 | 0.02514371 | 0.025143709 |
| 2481 | 1 | 29508727 | 29508887 | 0.02510467 | 0.025104672 |
| 2482 | 19 | 53935671 | 53935825 | 0.02503503 | 0.025035029 |
| 2483 | 19 | 49314114 | 49314260 | −0.0250238 | 0.0250238 |
| 2484 | 1 | 7187954 | 7188096 | −0.0249866 | 0.0249866 |
| 2485 | 1 | 151031997 | 151032151 | −0.0249441 | 0.0249441 |
| 2486 | 17 | 20870342 | 20870492 | 0.02486758 | 0.02486758 |
| 2487 | 17 | 80057178 | 80057427 | −0.0248171 | 0.0248171 |
| 2488 | 1 | 17306889 | 17307042 | 0.02470394 | 0.024703938 |
| 2489 | 3 | 128417280 | 128417387 | 0.02466356 | 0.024663561 |
| 2490 | 7 | 100137022 | 100137176 | 0.02460081 | 0.024600808 |
| 2491 | 10 | 8079936 | 8080088 | −0.0245944 | 0.0245944 |
| 2492 | 16 | 18995324 | 18995611 | −0.0245716 | 0.0245716 |
| 2493 | 10 | 112836105 | 112836245 | −0.0245433 | 0.0245433 |
| 2494 | 6 | 53516635 | 53516776 | 0.02450929 | 0.024509294 |
| 2495 | 3 | 150803981 | 150804124 | 0.02447865 | 0.024478655 |
| 2496 | 1 | 47900180 | 47900355 | −0.0244707 | 0.0244707 |
| 2497 | 1 | 210502426 | 210502567 | −0.0244332 | 0.0244332 |
| 2498 | 6 | 90121758 | 90121892 | 0.02442249 | 0.024422485 |
| 2499 | 1 | 249153042 | 249153212 | −0.0244122 | 0.0244122 |
| 2500 | 19 | 4791240 | 4791381 | −0.0244096 | 0.0244096 |
| 2501 | 21 | 45662186 | 45662327 | 0.02429253 | 0.024292532 |
| 2502 | 20 | 18547924 | 18548181 | 0.02426861 | 0.024268613 |
| 2503 | 20 | 10414643 | 10414802 | 0.02426527 | 0.024265272 |
| 2504 | 3 | 184080736 | 184080873 | −0.0242458 | 0.0242458 |
| 2505 | 11 | 61659251 | 61659508 | −0.0240574 | 0.0240574 |
| 2506 | 1 | 84971602 | 84971765 | 0.02403587 | 0.024035874 |
| 2507 | 11 | 19798664 | 19798825 | 0.02402279 | 0.024022791 |
| 2508 | 3 | 147137192 | 147137393 | −0.0240189 | 0.0240189 |
| 2509 | 5 | 160974475 | 160974622 | 0.02400835 | 0.024008346 |
| 2510 | 2 | 97523780 | 97523916 | −0.023943 | 0.023943 |
| 2511 | 16 | 279153 | 279303 | 0.02393229 | 0.023932286 |
| 2512 | 8 | 17779920 | 17780370 | −0.0239162 | 0.0239162 |
| 2513 | 3 | 133614413 | 133614649 | −0.0239041 | 0.0239041 |
| 2514 | 1 | 198125321 | 198125463 | −0.023859 | 0.023859 |
| 2515 | 2 | 119981690 | 119981837 | −0.0238487 | 0.0238487 |
| 2516 | 13 | 77901401 | 77901561 | −0.0237947 | 0.0237947 |
| 2517 | 12 | 56523196 | 56523352 | 0.02358849 | 0.023588485 |
| 2518 | 19 | 53237578 | 53237705 | 0.02356801 | 0.023568014 |
| 2519 | 1 | 65885647 | 65885769 | 0.02346075 | 0.023460753 |
| 2520 | 19 | 38879632 | 38879761 | −0.0234514 | 0.0234514 |
| 2521 | 1 | 111505942 | 111506111 | 0.02345047 | 0.023450473 |
| 2522 | 13 | 76055957 | 76056095 | −0.0233882 | 0.0233882 |
| 2523 | 13 | 39611840 | 39612164 | 0.0233848 | 0.023384801 |
| 2524 | 10 | 61469673 | 61469921 | 0.02335299 | 0.02335299 |
| 2525 | 1 | 223316399 | 223316735 | −0.0233472 | 0.0233472 |
| 2526 | 11 | 70115975 | 70116132 | 0.023341 | 0.023340999 |
| 2527 | 2 | 30142915 | 30143069 | −0.0232809 | 0.0232809 |
| 2528 | 3 | 52231772 | 52231912 | −0.0232581 | 0.0232581 |
| 2529 | 1 | 169337792 | 169337929 | −0.0232199 | 0.0232199 |
| 2530 | 17 | 58227217 | 58227332 | 0.02321444 | 0.023214442 |
| 2531 | 9 | 108147730 | 108148044 | 0.02319656 | 0.023196564 |
| 2532 | 18 | 18822499 | 18822647 | −0.023192 | 0.023192 |
| 2533 | 6 | 35887634 | 35887916 | 0.0231644 | 0.023164396 |
| 2534 | 1 | 16176306 | 16176463 | 0.02314221 | 0.02314221 |
| 2535 | 8 | 54794553 | 54794700 | 0.02313653 | 0.023136526 |
| 2536 | 3 | 28390598 | 28390707 | 0.02310647 | 0.023106469 |
| 2537 | 3 | 40351670 | 40351825 | 0.02306013 | 0.023060127 |
| 2538 | 10 | 121652127 | 121652460 | −0.0230201 | 0.0230201 |
| 2539 | 19 | 40724231 | 40724438 | 0.02294129 | 0.02294129 |
| 2540 | 15 | 66085018 | 66085170 | 0.02293335 | 0.022933349 |
| 2541 | 5 | 2754724 | 2756213 | −0.0229047 | 0.0229047 |
| 2542 | 7 | 65579862 | 65580003 | 0.02288654 | 0.022886542 |
| 2543 | 11 | 66511840 | 66512124 | 0.02280124 | 0.02280124 |
| 2544 | 7 | 84814578 | 84814635 | 0.02279731 | 0.022797314 |
| 2545 | 1 | 33283025 | 33283145 | −0.0227932 | 0.0227932 |
| 2546 | 8 | 134308967 | 134309123 | −0.0227727 | 0.0227727 |
| 2547 | 4 | 57843708 | 57843917 | −0.0227723 | 0.0227723 |
| 2548 | 21 | 46128759 | 46129714 | −0.0227439 | 0.0227439 |
| 2549 | 9 | 140024040 | 140024204 | 0.02272372 | 0.022723718 |
| 2550 | 16 | 21289689 | 21289904 | 0.0227166 | 0.022716603 |
| 2551 | 22 | 21356238 | 21356387 | −0.0227016 | 0.0227016 |
| 2552 | 15 | 72612551 | 72612933 | 0.02267795 | 0.022677952 |
| 2553 | 12 | 16035081 | 16035358 | 0.02263003 | 0.022630033 |
| 2554 | 11 | 121322557 | 121322793 | 0.02261976 | 0.022619761 |
| 2555 | 1 | 100998321 | 100998487 | 0.02252037 | 0.022520368 |
| 2556 | 18 | 30050251 | 30050472 | 0.02250917 | 0.022509165 |
| 2557 | 11 | 63637203 | 63637442 | −0.0224615 | 0.0224615 |
| 2558 | 1 | 151810418 | 151811675 | −0.02237 | 0.02237 |
| 2559 | 6 | 43603576 | 43603724 | −0.022352 | 0.022352 |
| 2560 | 12 | 109915324 | 109915540 | −0.0223302 | 0.0223302 |
| 2561 | 8 | 87521033 | 87521260 | 0.02232103 | 0.022321028 |
| 2562 | 7 | 105162520 | 105162775 | 0.02231012 | 0.022310118 |
| 2563 | 11 | 65819990 | 65820135 | 0.02226621 | 0.02226621 |
| 2564 | 17 | 36508845 | 36509000 | 0.02225937 | 0.022259373 |
| 2565 | 1 | 47133873 | 47134010 | −0.0222462 | 0.0222462 |
| 2566 | 16 | 23568327 | 23568893 | −0.0222364 | 0.0222364 |
| 2567 | 8 | 67025843 | 67026052 | 0.02221941 | 0.022219415 |
| 2568 | 21 | 36418125 | 36418949 | −0.0222162 | 0.0222162 |
| 2569 | 1 | 184005920 | 184006056 | −0.0221432 | 0.0221432 |
| 2570 | 16 | 28936976 | 28937096 | 0.02213923 | 0.022139225 |
| 2571 | 1 | 42500945 | 42501104 | 0.02207815 | 0.022078146 |
| 2572 | 1 | 39457021 | 39457271 | −0.0220738 | 0.0220738 |
| 2573 | 6 | 40554658 | 40554796 | −0.0220638 | 0.0220638 |
| 2574 | 4 | 184644247 | 184644388 | 0.02199367 | 0.021993667 |
| 2575 | 12 | 67909525 | 67909758 | 0.02197084 | 0.021970842 |
| 2576 | 19 | 36869953 | 36870253 | 0.02194781 | 0.021947806 |
| 2577 | 6 | 71998306 | 71998446 | −0.0219023 | 0.0219023 |
| 2578 | 2 | 216946320 | 216946453 | 0.02185418 | 0.02185418 |
| 2579 | 9 | 139010414 | 139010561 | −0.0218324 | 0.0218324 |
| 2580 | 2 | 190445398 | 190445632 | −0.0218173 | 0.0218173 |
| 2581 | 1 | 147806359 | 147806509 | 0.02179815 | 0.021798155 |
| 2582 | 4 | 4858408 | 4858563 | 0.02179152 | 0.021791518 |
| 2583 | 1 | 101713590 | 101713748 | 0.02178143 | 0.021781425 |
| 2584 | 8 | 54934895 | 54935150 | 0.02173994 | 0.021739942 |
| 2585 | 15 | 79724437 | 79724583 | 0.02167896 | 0.021678963 |
| 2586 | 16 | 30366214 | 30366379 | −0.0216687 | 0.0216687 |
| 2587 | 9 | 133329055 | 133329191 | −0.0216412 | 0.0216412 |
| 2588 | 17 | 64188323 | 64188463 | −0.0216408 | 0.0216408 |
| 2589 | 14 | 35885318 | 35885531 | 0.02161838 | 0.02161838 |
| 2590 | 11 | 4116372 | 4116516 | −0.0216136 | 0.0216136 |
| 2591 | 16 | 11031414 | 11031593 | 0.02159628 | 0.021596278 |
| 2592 | 14 | 75988403 | 75989233 | 0.02158948 | 0.021589476 |
| 2593 | 6 | 49430570 | 49431461 | −0.0215858 | 0.0215858 |
| 2594 | 20 | 277998 | 278130 | 0.02157904 | 0.021579044 |
| 2595 | 15 | 49913392 | 49913658 | −0.0215339 | 0.0215339 |
| 2596 | 17 | 28431689 | 28431969 | 0.02152368 | 0.021523676 |
| 2597 | 12 | 50898870 | 50899026 | 0.0214898 | 0.021489799 |
| 2598 | 4 | 111532608 | 111532744 | −0.0214339 | 0.0214339 |
| 2599 | 17 | 76125198 | 76125276 | 0.02139224 | 0.021392243 |
| 2600 | 8 | 74888998 | 74889133 | −0.0213896 | 0.0213896 |
| 2601 | 5 | 75843299 | 75843545 | −0.0213826 | 0.0213826 |
| 2602 | 16 | 2802113 | 2802463 | 0.0213728 | 0.021372795 |
| 2603 | 19 | 1847756 | 1847898 | 0.02134801 | 0.021348005 |
| 2604 | 11 | 67159041 | 67159194 | −0.0213249 | 0.0213249 |
| 2605 | 10 | 44101735 | 44102091 | −0.0213168 | 0.0213168 |
| 2606 | 12 | 22777951 | 22778160 | 0.0213025 | 0.021302499 |
| 2607 | 4 | 84256293 | 84256437 | −0.0211884 | 0.0211884 |
| 2608 | 6 | 1606302 | 1606438 | −0.021165 | 0.021165 |
| 2609 | 2 | 55845016 | 55845160 | 0.02112823 | 0.021128231 |
| 2610 | 1 | 48463226 | 48463368 | 0.02112134 | 0.021121338 |
| 2611 | 7 | 138145838 | 138145984 | −0.0211172 | 0.0211172 |
| 2612 | 6 | 52284727 | 52285045 | 0.02102885 | 0.021028851 |
| 2613 | 19 | 40030576 | 40030730 | −0.0210116 | 0.0210116 |
| 2614 | 8 | 101321544 | 101321690 | 0.02098044 | 0.020980436 |
| 2615 | 1 | 67895245 | 67895391 | 0.02092885 | 0.020928851 |
| 2616 | 10 | 98273634 | 98273788 | 0.02091535 | 0.020915345 |
| 2617 | 3 | 48481338 | 48481478 | −0.0209147 | 0.0209147 |
| 2618 | 5 | 76011224 | 76011377 | 0.02090658 | 0.020906584 |
| 2619 | 14 | 31889561 | 31889700 | −0.0208997 | 0.0208997 |
| 2620 | 1 | 115323051 | 115323203 | 0.02089169 | 0.020891695 |
| 2621 | 19 | 34012660 | 34013030 | 0.02085623 | 0.02085623 |
| 2622 | 2 | 210867205 | 210867416 | −0.0208481 | 0.0208481 |
| 2623 | 19 | 47551770 | 47552066 | −0.0208429 | 0.0208429 |
| 2624 | 6 | 36561874 | 36562055 | 0.02080695 | 0.02080695 |
| 2625 | 2 | 122494824 | 122494933 | −0.0207968 | 0.0207968 |
| 2626 | 5 | 156693501 | 156693647 | −0.0207841 | 0.0207841 |
| 2627 | 2 | 201729263 | 201729485 | −0.0207173 | 0.0207173 |
| 2628 | 15 | 91643464 | 91643613 | 0.02065521 | 0.020655214 |
| 2629 | 14 | 23067899 | 23068029 | −0.0205555 | 0.0205555 |
| 2630 | 16 | 86912390 | 86912706 | −0.0205474 | 0.0205474 |
| 2631 | 19 | 54982653 | 54982793 | −0.0205259 | 0.0205259 |
| 2632 | 2 | 115920397 | 115920548 | 0.02048347 | 0.020483466 |
| 2633 | 1 | 110692916 | 110693079 | −0.020471 | 0.020471 |
| 2634 | 14 | 89021398 | 89021536 | −0.0203769 | 0.0203769 |
| 2635 | 14 | 24584407 | 24584537 | 0.02036811 | 0.020368112 |
| 2636 | 2 | 26915814 | 26915964 | 0.02036723 | 0.020367225 |
| 2637 | 5 | 40486623 | 40486761 | −0.0203403 | 0.0203403 |
| 2638 | 8 | 42473948 | 42474217 | −0.0202476 | 0.0202476 |
| 2639 | 11 | 121593193 | 121593874 | −0.0202453 | 0.0202453 |
| 2640 | 11 | 64059228 | 64059664 | −0.0202436 | 0.0202436 |
| 2641 | 1 | 212587605 | 212587750 | −0.0202228 | 0.0202228 |
| 2642 | 5 | 157078662 | 157079115 | 0.020195 | 0.020194999 |
| 2643 | 4 | 168155057 | 168155191 | 0.0201572 | 0.020157203 |
| 2644 | 1 | 165667374 | 165667651 | 0.02014894 | 0.020148938 |
| 2645 | 9 | 6413775 | 6413923 | −0.0201268 | 0.0201268 |
| 2646 | 6 | 28626591 | 28626915 | 0.02004861 | 0.02004861 |
| 2647 | 3 | 44726832 | 44727262 | −0.0200478 | 0.0200478 |
| 2648 | 1 | 108508476 | 108508608 | 0.02003031 | 0.020030312 |
| 2649 | 5 | 171169396 | 171169565 | 0.01997537 | 0.019975367 |
| 2650 | 1 | 169862432 | 169862578 | −0.0199749 | 0.0199749 |
| 2651 | 1 | 155051086 | 155051234 | 0.01994095 | 0.019940955 |
| 2652 | 3 | 101497781 | 101498048 | 0.01992136 | 0.019921356 |
| 2653 | 3 | 71114691 | 71114839 | −0.0199149 | 0.0199149 |
| 2654 | 1 | 245132963 | 245133115 | 0.01987872 | 0.019878723 |
| 2655 | 17 | 7037013 | 7037159 | 0.01987496 | 0.019874961 |
| 2656 | 10 | 26727847 | 26727994 | −0.0198616 | 0.0198616 |
| 2657 | 15 | 96870152 | 96870298 | 0.01986072 | 0.019860716 |
| 2658 | 1 | 12226857 | 12227028 | 0.01981914 | 0.019819136 |
| 2659 | 22 | 38379427 | 38379575 | 0.01976113 | 0.019761127 |
| 2660 | 4 | 83955878 | 83956139 | −0.0197597 | 0.0197597 |
| 2661 | 1 | 33116426 | 33116566 | −0.0197462 | 0.0197462 |
| 2662 | 12 | 46662854 | 46663021 | 0.01969436 | 0.01969436 |
| 2663 | 3 | 12910147 | 12910535 | −0.019669 | 0.019669 |
| 2664 | 1 | 153931084 | 153931219 | −0.0196689 | 0.0196689 |
| 2665 | 5 | 37839849 | 37839995 | −0.0196431 | 0.0196431 |
| 2666 | 7 | 68895437 | 68895749 | −0.0196319 | 0.0196319 |
| 2667 | 22 | 24038320 | 24038966 | −0.0195989 | 0.0195989 |
| 2668 | 22 | 17566738 | 17567019 | 0.0195843 | 0.0195843 |
| 2669 | 6 | 2793935 | 2794113 | −0.0195821 | 0.0195821 |
| 2670 | 2 | 26257709 | 26257868 | −0.0194284 | 0.0194284 |
| 2671 | 19 | 38184192 | 38184339 | −0.0194175 | 0.0194175 |
| 2672 | 14 | 91716335 | 91716687 | −0.0193566 | 0.0193566 |
| 2673 | 14 | 100149888 | 100150069 | −0.0193244 | 0.0193244 |
| 2674 | 1 | 151119447 | 151119605 | 0.01927013 | 0.019270125 |
| 2675 | 1 | 201475873 | 201476015 | 0.0192485 | 0.019248495 |
| 2676 | 1 | 145209863 | 145209986 | 0.01924597 | 0.01924597 |
| 2677 | 6 | 26273517 | 26273615 | 0.01924396 | 0.01924396 |
| 2678 | 4 | 106068527 | 106068670 | 0.01918998 | 0.019189981 |
| 2679 | 10 | 81107020 | 81107333 | −0.0191421 | 0.0191421 |
| 2680 | 16 | 2255810 | 2255951 | −0.0191042 | 0.0191042 |
| 2681 | 1 | 52083118 | 52083265 | 0.01906849 | 0.019068491 |
| 2682 | 14 | 98190662 | 98191119 | −0.0190021 | 0.0190021 |
| 2683 | 7 | 140753500 | 140753673 | 0.01898333 | 0.018983335 |
| 2684 | 4 | 174255054 | 174255186 | −0.0189747 | 0.0189747 |
| 2685 | 2 | 172379547 | 172379691 | 0.0189382 | 0.018938201 |
| 2686 | 20 | 35491632 | 35491911 | 0.01893023 | 0.018930234 |
| 2687 | 17 | 80292648 | 80292780 | −0.0188871 | 0.0188871 |
| 2688 | 5 | 122372258 | 122372397 | 0.01888632 | 0.018886317 |
| 2689 | 17 | 79603688 | 79603834 | 0.01886997 | 0.018869972 |
| 2690 | 10 | 35625925 | 35626055 | −0.0188112 | 0.0188112 |
| 2691 | 19 | 48281785 | 48281931 | −0.0188087 | 0.0188087 |
| 2692 | 4 | 140004994 | 140005145 | −0.0187507 | 0.0187507 |
| 2693 | 9 | 125026890 | 125027134 | 0.01873697 | 0.018736971 |
| 2694 | 1 | 236230208 | 236230363 | −0.0187363 | 0.0187363 |
| 2695 | 6 | 70000086 | 70000358 | 0.01872869 | 0.018728692 |
| 2696 | 7 | 117824338 | 117824481 | 0.01872633 | 0.018726331 |
| 2697 | 18 | 74779338 | 74779666 | 0.01871866 | 0.018718659 |
| 2698 | 9 | 91136350 | 91136498 | 0.01869856 | 0.01869856 |
| 2699 | 14 | 23058034 | 23058214 | 0.01862606 | 0.018626056 |
| 2700 | 17 | 20811780 | 20811982 | −0.0184939 | 0.0184939 |
| 2701 | 11 | 75999234 | 75999401 | 0.01848257 | 0.018482573 |
| 2702 | 21 | 43092039 | 43092242 | −0.0184715 | 0.0184715 |
| 2703 | 3 | 131753789 | 131753928 | 0.01842236 | 0.018422362 |
| 2704 | 5 | 43064829 | 43064973 | −0.0184109 | 0.0184109 |
| 2705 | 5 | 88179350 | 88179456 | 0.01837849 | 0.018378492 |
| 2706 | 19 | 16308865 | 16308991 | −0.0183523 | 0.0183523 |
| 2707 | 12 | 104457815 | 104457962 | −0.0183376 | 0.0183376 |
| 2708 | 20 | 55841966 | 55842431 | −0.0182857 | 0.0182857 |
| 2709 | 9 | 3181583 | 3181745 | −0.0182724 | 0.0182724 |
| 2710 | 3 | 150127476 | 150127626 | −0.0182556 | 0.0182556 |
| 2711 | 3 | 121553752 | 121553852 | 0.01824866 | 0.018248658 |
| 2712 | 2 | 144695177 | 144695327 | 0.01820483 | 0.018204835 |
| 2713 | 14 | 70079314 | 70079472 | −0.0181713 | 0.0181713 |
| 2714 | 16 | 81040311 | 81040454 | 0.01809523 | 0.01809523 |
| 2715 | 3 | 150920932 | 150921088 | 0.01806318 | 0.018063181 |
| 2716 | 12 | 105089565 | 105089703 | 0.01805487 | 0.01805487 |
| 2717 | 3 | 75484134 | 75484244 | 0.01804936 | 0.018049365 |
| 2718 | 10 | 72141846 | 72141991 | 0.01804517 | 0.018045165 |
| 2719 | 10 | 38146447 | 38146658 | 0.01797908 | 0.017979077 |
| 2720 | 6 | 112194619 | 112194932 | −0.0179765 | 0.0179765 |
| 2721 | 11 | 111385416 | 111385561 | 0.01796689 | 0.017966889 |
| 2722 | 1 | 203274624 | 203274764 | 0.01795507 | 0.017955069 |
| 2723 | 3 | 101568966 | 101569114 | −0.0178921 | 0.0178921 |
| 2724 | 20 | 20432999 | 20433391 | −0.0178811 | 0.0178811 |
| 2725 | 11 | 61129195 | 61129511 | 0.01787669 | 0.017876686 |
| 2726 | 11 | 119455424 | 119455702 | 0.01786907 | 0.017869071 |
| 2727 | 7 | 99036419 | 99036592 | 0.01786881 | 0.017868805 |
| 2728 | 9 | 2015559 | 2015693 | −0.0177815 | 0.0177815 |
| 2729 | 16 | 89787410 | 89787603 | 0.01770711 | 0.017707113 |
| 2730 | 1 | 37980136 | 37980660 | 0.01767779 | 0.017677787 |
| 2731 | 1 | 93913397 | 93913540 | −0.01766 | 0.01766 |
| 2732 | 17 | 46724059 | 46724321 | 0.01763011 | 0.017630107 |
| 2733 | X | 117959949 | 117960082 | 0.01762502 | 0.017625023 |
| 2734 | 12 | 123874378 | 123874523 | 0.01760501 | 0.017605013 |
| 2735 | 1 | 113008821 | 113009061 | 0.01760072 | 0.017600718 |
| 2736 | 19 | 53496948 | 53497102 | −0.0176003 | 0.0176003 |
| 2737 | 2 | 154335108 | 154335261 | 0.01759629 | 0.017596285 |
| 2738 | 5 | 122111278 | 122111418 | 0.01756211 | 0.017562112 |
| 2739 | 17 | 30335662 | 30335810 | −0.0175376 | 0.0175376 |
| 2740 | 6 | 17281723 | 17281908 | −0.0174915 | 0.0174915 |
| 2741 | 16 | 89985085 | 89985230 | −0.0174882 | 0.0174882 |
| 2742 | 1 | 24104548 | 24104724 | 0.01745725 | 0.017457254 |
| 2743 | 1 | 207494518 | 207494800 | 0.01744095 | 0.017440949 |
| 2744 | 15 | 49912943 | 49913089 | −0.0173522 | 0.0173522 |
| 2745 | 8 | 23267968 | 23268121 | −0.0172833 | 0.0172833 |
| 2746 | 17 | 80023363 | 80023508 | 0.01727894 | 0.017278936 |
| 2747 | 9 | 100614897 | 100615487 | −0.017267 | 0.017267 |
| 2748 | 12 | 4647551 | 4647693 | 0.01725164 | 0.017251636 |
| 2749 | 3 | 9691736 | 9691881 | −0.0172488 | 0.0172488 |
| 2750 | 17 | 45867261 | 45867399 | 0.01722136 | 0.017221362 |
| 2751 | 17 | 47841508 | 47841609 | −0.0172146 | 0.0172146 |
| 2752 | 12 | 113012958 | 113013417 | −0.0171861 | 0.0171861 |
| 2753 | 16 | 17087359 | 17087575 | 0.01702782 | 0.01702782 |
| 2754 | 11 | 70244625 | 70244759 | −0.0170085 | 0.0170085 |
| 2755 | 2 | 113522129 | 113522274 | −0.0169979 | 0.0169979 |
| 2756 | 12 | 125478391 | 125478529 | −0.016977 | 0.016977 |
| 2757 | 15 | 101817837 | 101817993 | 0.01697107 | 0.016971073 |
| 2758 | 12 | 128850423 | 128850782 | −0.0169542 | 0.0169542 |
| 2759 | 17 | 80358725 | 80358976 | −0.0169186 | 0.0169186 |
| 2760 | 4 | 184319955 | 184320103 | 0.0169106 | 0.016910603 |
| 2761 | 11 | 16760610 | 16760750 | −0.016881 | 0.016881 |
| 2762 | 17 | 71287256 | 71287389 | −0.016822 | 0.016822 |
| 2763 | 22 | 50683418 | 50683632 | 0.0168056 | 0.016805597 |
| 2764 | 17 | 37353578 | 37353721 | 0.01676793 | 0.016767933 |
| 2765 | 5 | 79032472 | 79032713 | −0.0167637 | 0.0167637 |
| 2766 | 15 | 74220176 | 74220315 | 0.01672382 | 0.01672382 |
| 2767 | 1 | 166845610 | 166845817 | −0.0166787 | 0.0166787 |
| 2768 | 11 | 2114372 | 2114654 | −0.0166673 | 0.0166673 |
| 2769 | 6 | 70506464 | 70506564 | 0.0166582 | 0.016658202 |
| 2770 | 11 | 30607883 | 30608061 | −0.0165643 | 0.0165643 |
| 2771 | X | 48327173 | 48327388 | 0.01653563 | 0.016535625 |
| 2772 | 7 | 127176826 | 127176964 | 0.01649775 | 0.016497748 |
| 2773 | 5 | 92930502 | 92931263 | −0.0164932 | 0.0164932 |
| 2774 | 1 | 236445146 | 236445281 | 0.01649039 | 0.01649039 |
| 2775 | 4 | 108641523 | 108641670 | −0.0164798 | 0.0164798 |
| 2776 | 18 | 12884437 | 12884595 | 0.01647476 | 0.016474762 |
| 2777 | 18 | 13136285 | 13137749 | −0.0164327 | 0.0164327 |
| 2778 | 2 | 118943187 | 118944212 | −0.0164268 | 0.0164268 |
| 2779 | 6 | 36808165 | 36808564 | −0.0164242 | 0.0164242 |
| 2780 | 3 | 119814043 | 119814189 | 0.01639928 | 0.016399281 |
| 2781 | 11 | 58826041 | 58826186 | 0.01636017 | 0.016360169 |
| 2782 | 5 | 141061891 | 141062013 | 0.01628918 | 0.016289177 |
| 2783 | 10 | 2967855 | 2968163 | −0.016288 | 0.016288 |
| 2784 | 12 | 15111641 | 15112036 | −0.0162734 | 0.0162734 |
| 2785 | 8 | 12516458 | 12516587 | 0.01627078 | 0.016270777 |
| 2786 | 20 | 54987051 | 54987549 | −0.0162592 | 0.0162592 |
| 2787 | 1 | 173793860 | 173794145 | −0.016202 | 0.016202 |
| 2788 | 14 | 64805578 | 64805723 | 0.01618509 | 0.016185086 |
| 2789 | 1 | 117209867 | 117210011 | 0.01618076 | 0.016180762 |
| 2790 | 9 | 83728438 | 83728775 | −0.016169 | 0.016169 |
| 2791 | 22 | 30753058 | 30753200 | 0.01614158 | 0.016141575 |
| 2792 | 1 | 70687005 | 70687210 | −0.0161327 | 0.0161327 |
| 2793 | 2 | 64067462 | 64067610 | 0.01609856 | 0.016098556 |
| 2794 | 1 | 34629479 | 34629622 | −0.0160943 | 0.0160943 |
| 2795 | 1 | 203830981 | 203831129 | 0.01608514 | 0.016085139 |
| 2796 | 10 | 72142226 | 72142368 | 0.01606241 | 0.016062414 |
| 2797 | 19 | 53141550 | 53141684 | 0.01605872 | 0.016058724 |
| 2798 | 2 | 73403095 | 73403243 | 0.01602628 | 0.016026284 |
| 2799 | 6 | 86159612 | 86159745 | −0.0159967 | 0.0159967 |
| 2800 | 11 | 67722832 | 67722976 | 0.01598466 | 0.015984665 |
| 2801 | 15 | 81594294 | 81594514 | −0.0159355 | 0.0159355 |
| 2802 | 10 | 124905586 | 124906186 | −0.0159344 | 0.0159344 |
| 2803 | 10 | 134973968 | 134974100 | −0.0159296 | 0.0159296 |
| 2804 | 1 | 90286215 | 90286405 | 0.01592672 | 0.01592672 |
| 2805 | 5 | 32211340 | 32211527 | −0.0158925 | 0.0158925 |
| 2806 | 1 | 167407480 | 167407680 | −0.0158581 | 0.0158581 |
| 2807 | 14 | 24020113 | 24020261 | 0.01585517 | 0.015855166 |
| 2808 | 13 | 41635283 | 41635432 | 0.01579994 | 0.015799938 |
| 2809 | 11 | 91957046 | 91957196 | 0.01570201 | 0.015702014 |
| 2810 | 2 | 182548731 | 182548882 | 0.01568994 | 0.015689936 |
| 2811 | 9 | 95993559 | 95993979 | −0.0156858 | 0.0156858 |
| 2812 | 17 | 58217277 | 58217428 | 0.01568002 | 0.015680021 |
| 2813 | 12 | 57623296 | 57623529 | −0.0156792 | 0.0156792 |
| 2814 | 2 | 210337183 | 210337327 | −0.0156737 | 0.0156737 |
| 2815 | 12 | 133287695 | 133287871 | 0.01565569 | 0.015655692 |
| 2816 | 7 | 121784309 | 121784454 | −0.0156063 | 0.0156063 |
| 2817 | 12 | 130684123 | 130684267 | −0.0156022 | 0.0156022 |
| 2818 | 19 | 42058230 | 42058358 | 0.01559837 | 0.015598373 |
| 2819 | 1 | 32292562 | 32292701 | −0.0155528 | 0.0155528 |
| 2820 | 19 | 40732056 | 40732432 | −0.015545 | 0.015545 |
| 2821 | 12 | 102513635 | 102513846 | 0.01552311 | 0.015523113 |
| 2822 | 12 | 110152073 | 110152248 | 0.01549407 | 0.015494072 |
| 2823 | 4 | 178363954 | 178364113 | −0.0154637 | 0.0154637 |
| 2824 | 19 | 4769556 | 4769677 | 0.01544751 | 0.015447513 |
| 2825 | 1 | 41847294 | 41847853 | −0.0154117 | 0.0154117 |
| 2826 | 8 | 145150315 | 145150454 | −0.0154026 | 0.0154026 |
| 2827 | 19 | 49140570 | 49140889 | 0.01537572 | 0.015375721 |
| 2828 | 6 | 112314157 | 112314306 | 0.0153705 | 0.015370501 |
| 2829 | 15 | 81595261 | 81595370 | 0.01536035 | 0.015360346 |
| 2830 | 20 | 33543404 | 33543836 | 0.01535164 | 0.015351641 |
| 2831 | 17 | 6659292 | 6659443 | 0.01534454 | 0.015344542 |
| 2832 | 11 | 116724577 | 116724762 | 0.01532065 | 0.015320647 |
| 2833 | 10 | 104192198 | 104192302 | −0.0153026 | 0.0153026 |
| 2834 | 12 | 46120541 | 46120696 | 0.01526976 | 0.015269762 |
| 2835 | 14 | 91883970 | 91884133 | 0.01523729 | 0.015237293 |
| 2836 | 8 | 67624680 | 67624815 | 0.01519778 | 0.015197784 |
| 2837 | 4 | 146100519 | 146100653 | −0.01518 | 0.01518 |
| 2838 | 6 | 70505949 | 70506107 | −0.0151785 | 0.0151785 |
| 2839 | 13 | 113597414 | 113597562 | −0.0151586 | 0.0151586 |
| 2840 | 11 | 13689409 | 13689706 | 0.01514344 | 0.015143439 |
| 2841 | 6 | 159240473 | 159240608 | 0.01512026 | 0.015120261 |
| 2842 | 17 | 28562605 | 28562753 | −0.0150921 | 0.0150921 |
| 2843 | 10 | 91011105 | 91011249 | 0.01504783 | 0.015047826 |
| 2844 | 12 | 31478565 | 31478704 | −0.0149802 | 0.0149802 |
| 2845 | 2 | 10262762 | 10262954 | 0.014972 | 0.014972004 |
| 2846 | 12 | 32908449 | 32908590 | 0.01496477 | 0.014964772 |
| 2847 | 1 | 150207060 | 150207353 | 0.01495595 | 0.014955949 |
| 2848 | 6 | 10694565 | 10694710 | −0.0149029 | 0.0149029 |
| 2849 | 21 | 30364978 | 30365115 | 0.01489682 | 0.014896823 |
| 2850 | 20 | 33999759 | 33999973 | 0.01489056 | 0.014890561 |
| 2851 | 21 | 38071832 | 38071965 | 0.01488621 | 0.01488621 |
| 2852 | 9 | 77702549 | 77702691 | 0.01486747 | 0.01486747 |
| 2853 | 1 | 46806485 | 46806632 | 0.01484237 | 0.014842368 |
| 2854 | 18 | 25029212 | 25029355 | 0.01483202 | 0.014832023 |
| 2855 | 6 | 82463696 | 82463851 | −0.014804 | 0.014804 |
| 2856 | 17 | 9142796 | 9143120 | −0.0147925 | 0.0147925 |
| 2857 | 1 | 40157010 | 40157306 | 0.01473519 | 0.014735193 |
| 2858 | 11 | 13032300 | 13032678 | −0.0147273 | 0.0147273 |
| 2859 | 19 | 58838136 | 58838506 | −0.0147094 | 0.0147094 |
| 2860 | 10 | 124220654 | 124220792 | 0.01470765 | 0.01470765 |
| 2861 | 14 | 21439627 | 21439756 | −0.0146889 | 0.0146889 |
| 2862 | 3 | 150126471 | 150126605 | 0.01467359 | 0.014673588 |
| 2863 | 12 | 9910761 | 9910897 | −0.0145682 | 0.0145682 |
| 2864 | 6 | 106565485 | 106565670 | −0.0145377 | 0.0145377 |
| 2865 | 11 | 61731024 | 61731156 | 0.01453565 | 0.014535653 |
| 2866 | 2 | 160143463 | 160143600 | 0.01452022 | 0.014520217 |
| 2867 | 16 | 14726923 | 14727069 | −0.0145181 | 0.0145181 |
| 2868 | 4 | 148653544 | 148653692 | 0.01450869 | 0.014508688 |
| 2869 | 12 | 48153048 | 48153183 | 0.01449345 | 0.014493448 |
| 2870 | 15 | 78326965 | 78327102 | 0.01448715 | 0.014487151 |
| 2871 | 2 | 64068782 | 64069072 | 0.01444836 | 0.014448357 |
| 2872 | 1 | 6418980 | 6419127 | −0.0144455 | 0.0144455 |
| 2873 | 8 | 84051827 | 84052000 | −0.0144289 | 0.0144289 |
| 2874 | 8 | 121821383 | 121821525 | −0.0144288 | 0.0144288 |
| 2875 | 13 | 114875969 | 114876412 | 0.01440439 | 0.014404385 |
| 2876 | 15 | 65426126 | 65426271 | 0.01439068 | 0.014390677 |
| 2877 | 12 | 125027865 | 125028008 | 0.01437335 | 0.014373354 |
| 2878 | 14 | 31926698 | 31926875 | −0.0143614 | 0.0143614 |
| 2879 | 3 | 171176830 | 171177025 | −0.0143135 | 0.0143135 |
| 2880 | 15 | 99602732 | 99602875 | 0.01428469 | 0.014284686 |
| 2881 | 18 | 19749464 | 19749612 | 0.01427225 | 0.014272247 |
| 2882 | 17 | 42907858 | 42908008 | −0.0142138 | 0.0142138 |
| 2883 | 3 | 31574228 | 31574342 | −0.0141456 | 0.0141456 |
| 2884 | 19 | 5791078 | 5791228 | −0.0141113 | 0.0141113 |
| 2885 | 12 | 123237105 | 123237251 | 0.01411125 | 0.014111251 |
| 2886 | 15 | 94530468 | 94530612 | 0.014094 | 0.014094 |
| 2887 | 4 | 84256131 | 84256279 | 0.01405509 | 0.014055091 |
| 2888 | 18 | 57574436 | 57574579 | 0.01404412 | 0.014044124 |
| 2889 | 8 | 94753053 | 94753342 | −0.0139335 | 0.0139335 |
| 2890 | 1 | 204042701 | 204042848 | 0.01387189 | 0.013871885 |
| 2891 | 19 | 36485755 | 36485842 | 0.01383742 | 0.013837417 |
| 2892 | 1 | 165797094 | 165797240 | 0.01382761 | 0.013827607 |
| 2893 | 20 | 62072532 | 62072901 | −0.0138197 | 0.0138197 |
| 2894 | 3 | 43732223 | 43732405 | −0.0138027 | 0.0138027 |
| 2895 | 17 | 42299082 | 42299245 | 0.0137242 | 0.013724197 |
| 2896 | 3 | 45635635 | 45635760 | −0.0137013 | 0.0137013 |
| 2897 | 1 | 183604433 | 183604584 | 0.01369331 | 0.013693315 |
| 2898 | 4 | 81256713 | 81256971 | −0.0136656 | 0.0136656 |
| 2899 | 17 | 58603348 | 58603490 | 0.01366347 | 0.013663472 |
| 2900 | 18 | 9118553 | 9118708 | −0.0136313 | 0.0136313 |
| 2901 | 2 | 96971119 | 96971256 | −0.0135521 | 0.0135521 |
| 2902 | 7 | 101071824 | 101071964 | −0.0135312 | 0.0135312 |
| 2903 | 2 | 25496319 | 25496464 | 0.01352145 | 0.013521448 |
| 2904 | 12 | 131200452 | 131200616 | 0.01348895 | 0.013488948 |
| 2905 | 14 | 37133011 | 37133365 | −0.0134873 | 0.0134873 |
| 2906 | 2 | 201374816 | 201374977 | −0.0134527 | 0.0134527 |
| 2907 | 1 | 247570786 | 247570938 | −0.0134305 | 0.0134305 |
| 2908 | 7 | 142494834 | 142495050 | −0.013324 | 0.013324 |
| 2909 | 1 | 91172073 | 91172798 | −0.0133138 | 0.0133138 |
| 2910 | 7 | 99746441 | 99746699 | −0.0133065 | 0.0133065 |
| 2911 | 17 | 7240450 | 7240718 | −0.0132581 | 0.0132581 |
| 2912 | 9 | 14346321 | 14346668 | −0.0132451 | 0.0132451 |
| 2913 | 4 | 141348227 | 141348367 | −0.0132098 | 0.0132098 |
| 2914 | 1 | 150552527 | 150552705 | −0.0132047 | 0.0132047 |
| 2915 | 12 | 132568909 | 132569032 | −0.0132 | 0.0132 |
| 2916 | 20 | 61557896 | 61558074 | −0.0131709 | 0.0131709 |
| 2917 | 3 | 132378526 | 132378674 | −0.0130323 | 0.0130323 |
| 2918 | 1 | 202777380 | 202777693 | 0.0129624 | 0.012962399 |
| 2919 | 11 | 67250548 | 67250694 | 0.01288823 | 0.012888227 |
| 2920 | 1 | 242947623 | 242947769 | 0.01285377 | 0.012853774 |
| 2921 | 6 | 90539724 | 90539870 | 0.01285363 | 0.012853625 |
| 2922 | 7 | 130080767 | 130080909 | −0.0128418 | 0.0128418 |
| 2923 | 20 | 55503332 | 55503472 | −0.0128149 | 0.0128149 |
| 2924 | 4 | 25162403 | 25162548 | −0.0127933 | 0.0127933 |
| 2925 | 15 | 62123384 | 62123541 | −0.0127503 | 0.0127503 |
| 2926 | 11 | 4628963 | 4629104 | 0.01267698 | 0.01267698 |
| 2927 | 3 | 113233977 | 113234142 | −0.012672 | 0.012672 |
| 2928 | 10 | 115439187 | 115439462 | 0.01266379 | 0.012663795 |
| 2929 | 3 | 98620494 | 98620742 | 0.01265166 | 0.012651658 |
| 2930 | 3 | 62359319 | 62359455 | −0.0126516 | 0.0126516 |
| 2931 | 11 | 77300485 | 77300647 | 0.01264039 | 0.012640388 |
| 2932 | 1 | 157125982 | 157126135 | 0.01262797 | 0.012627969 |
| 2933 | 7 | 92861702 | 92861908 | −0.0126277 | 0.0126277 |
| 2934 | 20 | 24451813 | 24451958 | −0.0125946 | 0.0125946 |
| 2935 | 19 | 7183007 | 7183221 | −0.0125745 | 0.0125745 |
| 2936 | 4 | 1161080 | 1161218 | 0.0125701 | 0.012570103 |
| 2937 | 4 | 68566367 | 68566515 | −0.0125655 | 0.0125655 |
| 2938 | 3 | 131221619 | 131221934 | 0.01256336 | 0.01256336 |
| 2939 | 17 | 42836675 | 42836799 | −0.0125287 | 0.0125287 |
| 2940 | 21 | 27542965 | 27543205 | 0.01251391 | 0.012513913 |
| 2941 | 11 | 12863803 | 12864020 | 0.01251174 | 0.012511745 |
| 2942 | 20 | 34824571 | 34824717 | 0.01250931 | 0.012509306 |
| 2943 | 10 | 94822837 | 94822976 | −0.0125061 | 0.0125061 |
| 2944 | 9 | 99382004 | 99382138 | −0.0124868 | 0.0124868 |
| 2945 | 17 | 1161898 | 1162142 | −0.012457 | 0.012457 |
| 2946 | 17 | 65713511 | 65713606 | 0.0124462 | 0.012446202 |
| 2947 | 8 | 120886765 | 120886911 | 0.01241499 | 0.012414992 |
| 2948 | 13 | 80916990 | 80917128 | 0.01236037 | 0.012360372 |
| 2949 | 7 | 30028336 | 30028478 | −0.012339 | 0.012339 |
| 2950 | 2 | 175191802 | 175192657 | −0.0123315 | 0.0123315 |
| 2951 | 14 | 102275953 | 102276097 | 0.01230992 | 0.012309916 |
| 2952 | 6 | 121758575 | 121758732 | 0.0123077 | 0.012307703 |
| 2953 | 6 | 17600680 | 17600819 | −0.0123072 | 0.0123072 |
| 2954 | 8 | 30013637 | 30013757 | −0.012295 | 0.012295 |
| 2955 | 2 | 165697943 | 165698179 | −0.0122851 | 0.0122851 |
| 2956 | 1 | 53017887 | 53018033 | 0.0122792 | 0.012279202 |
| 2957 | 14 | 105559478 | 105559635 | 0.01226005 | 0.01226005 |
| 2958 | 19 | 2273764 | 2273910 | 0.01223183 | 0.012231827 |
| 2959 | 8 | 133787846 | 133787993 | 0.01218939 | 0.012189393 |
| 2960 | 2 | 163027449 | 163027598 | −0.0121172 | 0.0121172 |
| 2961 | 8 | 126010390 | 126010537 | −0.0120836 | 0.0120836 |
| 2962 | 19 | 36705897 | 36706164 | 0.01206489 | 0.012064886 |
| 2963 | 1 | 160832528 | 160832688 | −0.0120639 | 0.0120639 |
| 2964 | 2 | 202097051 | 202097200 | −0.0120155 | 0.0120155 |
| 2965 | 4 | 9783114 | 9783468 | −0.0119573 | 0.0119573 |
| 2966 | 7 | 4910958 | 4911105 | −0.0119502 | 0.0119502 |
| 2967 | 20 | 1014652 | 1014806 | −0.0119454 | 0.0119454 |
| 2968 | 18 | 54318245 | 54318575 | −0.011915 | 0.011915 |
| 2969 | 11 | 72145613 | 72145762 | −0.0119097 | 0.0119097 |
| 2970 | 17 | 73663330 | 73663636 | −0.011864 | 0.011864 |
| 2971 | 5 | 78280803 | 78280943 | −0.0118482 | 0.0118482 |
| 2972 | 3 | 178865609 | 178865762 | −0.0118474 | 0.0118474 |
| 2973 | 1 | 245605032 | 245605341 | −0.0118302 | 0.0118302 |
| 2974 | 1 | 27960512 | 27960651 | 0.01182747 | 0.011827473 |
| 2975 | 3 | 133392864 | 133393636 | −0.0117542 | 0.0117542 |
| 2976 | 11 | 110581803 | 110581940 | −0.0117121 | 0.0117121 |
| 2977 | 5 | 87976215 | 87976356 | −0.0117037 | 0.0117037 |
| 2978 | 6 | 91296590 | 91296855 | −0.0116926 | 0.0116926 |
| 2979 | 5 | 121297432 | 121297749 | −0.0116777 | 0.0116777 |
| 2980 | 5 | 162931335 | 162931970 | −0.0116639 | 0.0116639 |
| 2981 | 12 | 72234236 | 72234385 | 0.01162996 | 0.011629959 |
| 2982 | 21 | 34444263 | 34444463 | 0.01162112 | 0.011621118 |
| 2983 | 15 | 63334937 | 63335106 | −0.0116176 | 0.0116176 |
| 2984 | 1 | 201708737 | 201709877 | −0.0115959 | 0.0115959 |
| 2985 | 14 | 71788041 | 71788187 | −0.0115617 | 0.0115617 |
| 2986 | 1 | 224804318 | 224804463 | 0.01145408 | 0.011454081 |
| 2987 | 7 | 27224793 | 27224935 | −0.0114439 | 0.0114439 |
| 2988 | 20 | 25062369 | 25062513 | −0.0114418 | 0.0114418 |
| 2989 | 14 | 31677141 | 31677358 | 0.01140987 | 0.011409872 |
| 2990 | 16 | 87814120 | 87814332 | 0.0113463 | 0.0113463 |
| 2991 | 22 | 44257942 | 44258395 | −0.0113239 | 0.0113239 |
| 2992 | 12 | 4382907 | 4383099 | 0.01130431 | 0.011304306 |
| 2993 | 19 | 13984012 | 13984158 | 0.01128216 | 0.011282157 |
| 2994 | 7 | 134331341 | 134331478 | −0.0111955 | 0.0111955 |
| 2995 | 14 | 89897129 | 89897438 | −0.0111329 | 0.0111329 |
| 2996 | 2 | 29234484 | 29234791 | −0.0111326 | 0.0111326 |
| 2997 | 1 | 3566408 | 3566629 | −0.0111184 | 0.0111184 |
| 2998 | 12 | 47473134 | 47473270 | −0.0110648 | 0.0110648 |
| 2999 | 19 | 36135683 | 36135833 | −0.0109403 | 0.0109403 |
| 3000 | 14 | 53618955 | 53619114 | 0.01092234 | 0.010922343 |
| 3001 | 8 | 5820983 | 5821136 | −0.0109106 | 0.0109106 |
| 3002 | 14 | 50065136 | 50065415 | 0.01091021 | 0.010910213 |
| 3003 | 4 | 57843464 | 57843622 | 0.01090102 | 0.010901023 |
| 3004 | 17 | 79988670 | 79988822 | 0.01089864 | 0.010898642 |
| 3005 | 1 | 227975007 | 227975151 | −0.0108833 | 0.0108833 |
| 3006 | 1 | 226187402 | 226187548 | 0.0108541 | 0.010854096 |
| 3007 | 3 | 49467109 | 49467259 | −0.0108512 | 0.0108512 |
| 3008 | 8 | 1978849 | 1978989 | 0.01083754 | 0.010837536 |
| 3009 | 2 | 230476489 | 230476632 | −0.010832 | 0.010832 |
| 3010 | 15 | 83735792 | 83735997 | 0.01080995 | 0.010809948 |
| 3011 | 6 | 28227316 | 28227487 | 0.01079459 | 0.010794592 |
| 3012 | 2 | 114261909 | 114262055 | 0.01076451 | 0.010764506 |
| 3013 | 12 | 113914431 | 113914577 | −0.0107066 | 0.0107066 |
| 3014 | 9 | 106856598 | 106856792 | −0.0106893 | 0.0106893 |
| 3015 | 1 | 235805573 | 235805926 | 0.01068829 | 0.010688286 |
| 3016 | 16 | 55514390 | 55514537 | −0.0106867 | 0.0106867 |
| 3017 | 4 | 109093138 | 109093298 | 0.01066571 | 0.010665715 |
| 3018 | 8 | 6899737 | 6899927 | −0.0105865 | 0.0105865 |
| 3019 | 9 | 14347243 | 14348104 | 0.01057781 | 0.010577808 |
| 3020 | 1 | 110754007 | 110754163 | 0.0105517 | 0.010551698 |
| 3021 | 1 | 5526593 | 5526803 | −0.0105325 | 0.0105325 |
| 3022 | 9 | 138852426 | 138852579 | −0.0105292 | 0.0105292 |
| 3023 | 8 | 67837612 | 67837751 | 0.01047289 | 0.010472886 |
| 3024 | 4 | 123843503 | 123843652 | 0.01044816 | 0.010448159 |
| 3025 | 19 | 18220612 | 18220698 | −0.0104091 | 0.0104091 |
| 3026 | 17 | 73782031 | 73782274 | −0.0103667 | 0.0103667 |
| 3027 | 1 | 19638334 | 19638479 | −0.0103541 | 0.0103541 |
| 3028 | 6 | 29944691 | 29944936 | 0.01034382 | 0.01034382 |
| 3029 | 5 | 98269155 | 98269395 | −0.010268 | 0.010268 |
| 3030 | 2 | 120770392 | 120770539 | −0.0102533 | 0.0102533 |
| 3031 | 6 | 26365142 | 26365273 | −0.0101961 | 0.0101961 |
| 3032 | 1 | 160951749 | 160952185 | −0.0101927 | 0.0101927 |
| 3033 | 16 | 18573142 | 18573251 | −0.0101473 | 0.0101473 |
| 3034 | 1 | 179923584 | 179923728 | −0.0101347 | 0.0101347 |
| 3035 | 14 | 68086805 | 68086952 | 0.01012744 | 0.010127441 |
| 3036 | 9 | 103235966 | 103236121 | −0.0100793 | 0.0100793 |
| 3037 | 1 | 26202361 | 26202513 | −0.0100522 | 0.0100522 |
| 3038 | 10 | 92617985 | 92618131 | −0.0100059 | 0.0100059 |
| 3039 | 1 | 155057401 | 155057510 | 0.00998632 | 0.009986317 |
| 3040 | 2 | 26467239 | 26467375 | −0.009936 | 0.00993605 |
| 3041 | 11 | 57298463 | 57298603 | 0.00992353 | 0.009923533 |
| 3042 | 1 | 59249731 | 59249911 | 0.00989535 | 0.009895352 |
| 3043 | 5 | 180687993 | 180688137 | −0.0098866 | 0.00988663 |
| 3044 | 12 | 104851831 | 104851978 | −0.0098861 | 0.00988607 |
| 3045 | 19 | 19322600 | 19322740 | −0.0098822 | 0.00988223 |
| 3046 | 14 | 68750295 | 68750440 | −0.0098761 | 0.0098761 |
| 3047 | 5 | 1315967 | 1316220 | 0.00987534 | 0.009875336 |
| 3048 | X | 94975778 | 94976025 | 0.00987476 | 0.009874757 |
| 3049 | 17 | 46810612 | 46810852 | −0.0098558 | 0.00985584 |
| 3050 | 8 | 6747866 | 6748119 | −0.0098558 | 0.00985583 |
| 3051 | 17 | 33815220 | 33815317 | 0.00984378 | 0.009843775 |
| 3052 | 16 | 4898174 | 4898311 | 0.00983513 | 0.009835135 |
| 3053 | 17 | 35293850 | 35293998 | −0.0098324 | 0.00983238 |
| 3054 | 21 | 41015412 | 41015576 | −0.0098087 | 0.00980868 |
| 3055 | 5 | 100236124 | 100236259 | 0.00976293 | 0.00976293 |
| 3056 | 1 | 181059082 | 181059228 | 0.00973908 | 0.009739083 |
| 3057 | 22 | 45329677 | 45330083 | 0.00964851 | 0.009648514 |
| 3058 | 16 | 14396080 | 14396205 | 0.00963598 | 0.009635984 |
| 3059 | 1 | 151254749 | 151254884 | 0.00963045 | 0.009630452 |
| 3060 | 15 | 71056058 | 71056210 | −0.0096226 | 0.00962257 |
| 3061 | 7 | 143105892 | 143106044 | −0.0095929 | 0.00959291 |
| 3062 | 4 | 17615925 | 17616073 | 0.00956176 | 0.009561762 |
| 3063 | 20 | 44935962 | 44936103 | −0.0095519 | 0.00955192 |
| 3064 | 11 | 3601986 | 3602087 | 0.00943717 | 0.009437168 |
| 3065 | 7 | 128045607 | 128045748 | 0.00943592 | 0.009435921 |
| 3066 | 11 | 18548130 | 18548274 | −0.0094256 | 0.00942561 |
| 3067 | 1 | 193029049 | 193029185 | 0.00942272 | 0.009422717 |
| 3068 | 1 | 224805683 | 224805839 | 0.00940001 | 0.009400006 |
| 3069 | 1 | 12239338 | 12239516 | 0.00938581 | 0.009385812 |
| 3070 | 16 | 5007763 | 5008041 | 0.00926483 | 0.00926483 |
| 3071 | 1 | 181998873 | 181999000 | −0.009263 | 0.00926302 |
| 3072 | 20 | 30327363 | 30327495 | −0.0092185 | 0.00921846 |
| 3073 | 7 | 93519933 | 93520243 | 0.00920678 | 0.009206781 |
| 3074 | 1 | 36863229 | 36863491 | 0.00919144 | 0.009191442 |
| 3075 | 2 | 171627643 | 171627860 | −0.0091598 | 0.00915981 |
| 3076 | 19 | 41903157 | 41903411 | −0.0090905 | 0.00909053 |
| 3077 | 7 | 99070303 | 99070657 | 0.00905333 | 0.009053335 |
| 3078 | 2 | 182542788 | 182542935 | 0.00904043 | 0.009040429 |
| 3079 | 6 | 107780720 | 107781079 | 0.00900461 | 0.009004605 |
| 3080 | 11 | 44972623 | 44972765 | 0.00894094 | 0.008940942 |
| 3081 | 20 | 55383355 | 55383481 | 0.00892165 | 0.008921649 |
| 3082 | 5 | 159739751 | 159739896 | 0.00891022 | 0.008910224 |
| 3083 | 1 | 111682513 | 111682734 | 0.0088889 | 0.008888895 |
| 3084 | 17 | 49231114 | 49231309 | 0.0088439 | 0.008843899 |
| 3085 | 11 | 7041579 | 7041761 | −0.0087993 | 0.00879925 |
| 3086 | 16 | 17510689 | 17510833 | −0.0087888 | 0.0087888 |
| 3087 | 10 | 31875809 | 31876008 | −0.0087558 | 0.0087558 |
| 3088 | 4 | 3957594 | 3957707 | −0.008752 | 0.00875196 |
| 3089 | 14 | 24665122 | 24665266 | 0.00872698 | 0.008726976 |
| 3090 | 12 | 54402651 | 54402788 | 0.00870884 | 0.008708837 |
| 3091 | 19 | 36236462 | 36236695 | −0.0086818 | 0.0086818 |
| 3092 | 4 | 57845323 | 57845467 | −0.0086786 | 0.00867861 |
| 3093 | 1 | 40840495 | 40840704 | −0.0086716 | 0.00867156 |
| 3094 | 11 | 93517231 | 93517481 | −0.0086672 | 0.00866718 |
| 3095 | 13 | 21635658 | 21635909 | 0.00866191 | 0.008661906 |
| 3096 | 10 | 129809748 | 129809885 | −0.0086533 | 0.00865326 |
| 3097 | X | 152089945 | 152090193 | 0.00860563 | 0.008605631 |
| 3098 | 19 | 39936540 | 39936733 | 0.00859009 | 0.008590088 |
| 3099 | 8 | 145652824 | 145653141 | 0.00857125 | 0.008571249 |
| 3100 | 9 | 77567284 | 77567565 | 0.00857044 | 0.008570441 |
| 3101 | 1 | 205425388 | 205425548 | −0.0085429 | 0.00854286 |
| 3102 | 12 | 133485611 | 133485754 | −0.0085248 | 0.00852479 |
| 3103 | 11 | 116063750 | 116063900 | 0.00851501 | 0.008515015 |
| 3104 | 1 | 85930396 | 85930545 | −0.0085076 | 0.00850758 |
| 3105 | X | 153541751 | 153541909 | −0.0084854 | 0.00848543 |
| 3106 | 6 | 30538765 | 30538908 | 0.00848202 | 0.008482024 |
| 3107 | 10 | 134600768 | 134600945 | −0.008437 | 0.00843698 |
| 3108 | 1 | 148555980 | 148556132 | 0.00838962 | 0.008389616 |
| 3109 | 6 | 144471355 | 144471634 | 0.00837979 | 0.008379794 |
| 3110 | 7 | 107220267 | 107220454 | −0.008363 | 0.00836301 |
| 3111 | 4 | 103997807 | 103997952 | 0.00835597 | 0.008355972 |
| 3112 | 17 | 19266364 | 19266501 | −0.008346 | 0.00834597 |
| 3113 | 11 | 63381754 | 63381892 | 0.00833587 | 0.008335867 |
| 3114 | 6 | 3068484 | 3068653 | 0.00828945 | 0.008289455 |
| 3115 | 1 | 201798532 | 201798690 | −0.0082798 | 0.00827981 |
| 3116 | 15 | 79382471 | 79382609 | −0.008225 | 0.00822502 |
| 3117 | 19 | 36138953 | 36139099 | −0.0082248 | 0.00822485 |
| 3118 | 1 | 207226526 | 207226911 | 0.00820058 | 0.008200584 |
| 3119 | 17 | 65471572 | 65471881 | −0.0081985 | 0.00819852 |
| 3120 | 12 | 122227158 | 122227564 | 0.00813169 | 0.008131694 |
| 3121 | X | 136650527 | 136650730 | 0.00812873 | 0.008128729 |
| 3122 | 9 | 111695652 | 111695803 | −0.0081232 | 0.00812325 |
| 3123 | 10 | 130339139 | 130339387 | 0.00812056 | 0.008120557 |
| 3124 | 5 | 170739071 | 170739944 | −0.0080906 | 0.00809064 |
| 3125 | 6 | 167276545 | 167276689 | −0.0080672 | 0.00806722 |
| 3126 | 17 | 57184109 | 57184545 | 0.00804472 | 0.008044722 |
| 3127 | 6 | 33385971 | 33386235 | −0.0079625 | 0.0079625 |
| 3128 | 3 | 45731140 | 45731322 | −0.00794 | 0.00793996 |
| 3129 | 2 | 190539332 | 190539571 | 0.00793427 | 0.007934268 |
| 3130 | 15 | 69687017 | 69687166 | −0.0079105 | 0.00791048 |
| 3131 | 12 | 45610615 | 45610789 | 0.00786273 | 0.007862732 |
| 3132 | 5 | 142150728 | 142150867 | −0.0078549 | 0.00785489 |
| 3133 | 19 | 17914074 | 17914449 | −0.0078516 | 0.00785157 |
| 3134 | 17 | 4407523 | 4407662 | −0.0078068 | 0.00780678 |
| 3135 | 7 | 71800643 | 71802299 | −0.0078005 | 0.0078005 |
| 3136 | 6 | 32939227 | 32939503 | 0.00776215 | 0.007762152 |
| 3137 | 17 | 25680546 | 25680691 | 0.00770509 | 0.007705094 |
| 3138 | 10 | 44847942 | 44848080 | −0.0076842 | 0.00768421 |
| 3139 | 3 | 184429322 | 184429464 | −0.0076804 | 0.00768038 |
| 3140 | 12 | 8233985 | 8234156 | −0.0076168 | 0.00761684 |
| 3141 | 3 | 52090054 | 52090230 | 0.00757208 | 0.007572083 |
| 3142 | 4 | 13546403 | 13546563 | −0.0075414 | 0.00754139 |
| 3143 | 6 | 27759309 | 27759463 | 0.00748737 | 0.007487375 |
| 3144 | 7 | 86849388 | 86849569 | −0.0074669 | 0.00746693 |
| 3145 | 16 | 2581758 | 2581900 | −0.007465 | 0.00746499 |
| 3146 | 3 | 58885157 | 58885461 | 0.0074165 | 0.007416503 |
| 3147 | 7 | 97736290 | 97736430 | 0.00740103 | 0.007401033 |
| 3148 | 16 | 54446071 | 54446283 | 0.00738191 | 0.007381908 |
| 3149 | 19 | 16677146 | 16677384 | −0.0073576 | 0.00735758 |
| 3150 | 6 | 33290364 | 33290503 | −0.0073393 | 0.00733926 |
| 3151 | 7 | 96746965 | 96747148 | 0.00729951 | 0.007299511 |
| 3152 | 11 | 18415774 | 18416050 | −0.007299 | 0.00729898 |
| 3153 | 6 | 20319993 | 20320512 | −0.0072891 | 0.00728911 |
| 3154 | 2 | 54785483 | 54785615 | 0.00724907 | 0.007249071 |
| 3155 | 1 | 156737381 | 156737522 | 0.00724586 | 0.007245857 |
| 3156 | 6 | 74098733 | 74098878 | 0.00724289 | 0.007242894 |
| 3157 | 2 | 218899542 | 218899663 | 0.00723382 | 0.007233821 |
| 3158 | 2 | 101768180 | 101768327 | −0.0072161 | 0.00721614 |
| 3159 | 21 | 43509532 | 43509673 | 0.00721019 | 0.007210187 |
| 3160 | 2 | 97304115 | 97304267 | 0.00720385 | 0.007203847 |
| 3161 | 7 | 2548140 | 2548292 | −0.0071957 | 0.00719565 |
| 3162 | 16 | 54318240 | 54318393 | −0.0071759 | 0.00717594 |
| 3163 | 1 | 201084040 | 201084185 | 0.00717442 | 0.007174416 |
| 3164 | 6 | 31830448 | 31830695 | −0.007155 | 0.00715496 |
| 3165 | 22 | 24407684 | 24407936 | 0.00710623 | 0.007106234 |
| 3166 | 11 | 123447772 | 123447911 | 0.00710084 | 0.007100844 |
| 3167 | 11 | 118799410 | 118799555 | −0.0070979 | 0.00709791 |
| 3168 | 1 | 43919784 | 43919930 | −0.0070938 | 0.0070938 |
| 3169 | 10 | 21785918 | 21786056 | −0.0070604 | 0.00706044 |
| 3170 | 12 | 52512468 | 52512608 | 0.00705214 | 0.007052137 |
| 3171 | 22 | 17717976 | 17718102 | −0.0069409 | 0.00694092 |
| 3172 | 19 | 984685 | 984820 | −0.0069253 | 0.00692528 |
| 3173 | 2 | 239229289 | 239229470 | 0.00692184 | 0.00692184 |
| 3174 | 2 | 217363386 | 217363535 | 0.00691113 | 0.006911128 |
| 3175 | 22 | 38214901 | 38215045 | 0.00690917 | 0.006909171 |
| 3176 | 11 | 11643683 | 11643884 | −0.0069022 | 0.00690217 |
| 3177 | 10 | 63663060 | 63663341 | 0.00687095 | 0.006870949 |
| 3178 | 1 | 26147221 | 26147430 | 0.00684624 | 0.006846239 |
| 3179 | 15 | 43477532 | 43477704 | −0.0068166 | 0.00681661 |
| 3180 | 7 | 19151938 | 19152453 | −0.0068093 | 0.00680926 |
| 3181 | 3 | 160939367 | 160939511 | −0.0068051 | 0.00680509 |
| 3182 | 6 | 10695040 | 10695138 | 0.0068011 | 0.006801099 |
| 3183 | 6 | 117583090 | 117583226 | −0.0068002 | 0.0068002 |
| 3184 | 4 | 108745967 | 108746124 | 0.00678576 | 0.006785756 |
| 3185 | 2 | 161350438 | 161350586 | 0.00676615 | 0.00676615 |
| 3186 | 7 | 127228146 | 127228294 | 0.00675578 | 0.006755782 |
| 3187 | 14 | 107154896 | 107155210 | −0.0067419 | 0.00674189 |
| 3188 | 17 | 4851353 | 4851491 | −0.0067028 | 0.0067028 |
| 3189 | X | 1601318 | 1601453 | 0.00665436 | 0.006654364 |
| 3190 | 14 | 52313965 | 52314420 | −0.0066503 | 0.00665028 |
| 3191 | 10 | 87354117 | 87354278 | 0.00664844 | 0.006648443 |
| 3192 | 17 | 40021817 | 40021929 | −0.0066054 | 0.00660544 |
| 3193 | 9 | 130213721 | 130213865 | −0.0065852 | 0.00658515 |
| 3194 | 5 | 174145117 | 174145360 | 0.00655338 | 0.006553376 |
| 3195 | 3 | 45151751 | 45151935 | 0.00653009 | 0.006530088 |
| 3196 | 21 | 38078962 | 38079238 | −0.0065214 | 0.00652137 |
| 3197 | 4 | 166673074 | 166673215 | −0.0065188 | 0.00651884 |
| 3198 | 19 | 8008504 | 8008751 | −0.0064014 | 0.00640143 |
| 3199 | 1 | 203830796 | 203830975 | −0.0063877 | 0.00638766 |
| 3200 | 6 | 17987155 | 17987299 | 0.0063662 | 0.006366203 |
| 3201 | 6 | 130702981 | 130703140 | −0.0062888 | 0.00628884 |
| 3202 | 20 | 49574858 | 49575009 | 0.00625866 | 0.006258657 |
| 3203 | 20 | 36322312 | 36322568 | 0.00623669 | 0.006236694 |
| 3204 | 19 | 11072078 | 11072214 | −0.0062073 | 0.00620727 |
| 3205 | 19 | 55866255 | 55866445 | −0.0061822 | 0.00618221 |
| 3206 | 2 | 154335268 | 154335452 | −0.006169 | 0.00616898 |
| 3207 | 16 | 1822956 | 1823105 | 0.00616607 | 0.006166069 |
| 3208 | 22 | 19419859 | 19420246 | −0.0061486 | 0.0061486 |
| 3209 | 1 | 24070332 | 24070475 | −0.0061458 | 0.00614583 |
| 3210 | 1 | 90287375 | 90287538 | −0.0061357 | 0.00613566 |
| 3211 | 14 | 93673106 | 93673373 | 0.00612959 | 0.006129586 |
| 3212 | 11 | 110582958 | 110583105 | 0.00609609 | 0.006096087 |
| 3213 | 1 | 31867530 | 31867773 | 0.00606399 | 0.006063987 |
| 3214 | 3 | 39093319 | 39093571 | 0.00605248 | 0.006052479 |
| 3215 | 16 | 11716374 | 11716509 | 0.00603364 | 0.006033643 |
| 3216 | 3 | 187461391 | 187461525 | 0.00594722 | 0.005947225 |
| 3217 | 4 | 44728564 | 44728805 | 0.00592606 | 0.005926065 |
| 3218 | 7 | 19748210 | 19748550 | −0.0058917 | 0.00589166 |
| 3219 | 6 | 31548766 | 31548944 | 0.00583597 | 0.005835969 |
| 3220 | 16 | 25118158 | 25118292 | 0.00582008 | 0.005820076 |
| 3221 | 12 | 56660550 | 56660658 | −0.0058111 | 0.00581105 |
| 3222 | 22 | 41698773 | 41698937 | 0.0057995 | 0.005799501 |
| 3223 | 9 | 132652895 | 132653168 | −0.0057862 | 0.00578618 |
| 3224 | 1 | 47904385 | 47904531 | −0.0057726 | 0.00577259 |
| 3225 | 19 | 38270374 | 38270521 | 0.00575756 | 0.005757557 |
| 3226 | 2 | 96987847 | 96987982 | −0.0057497 | 0.00574971 |
| 3227 | 11 | 129244365 | 129244511 | 0.00574188 | 0.005741875 |
| 3228 | 1 | 1840405 | 1840522 | −0.0057216 | 0.00572156 |
| 3229 | 5 | 175793413 | 175793561 | 0.00571679 | 0.00571679 |
| 3230 | 17 | 29638710 | 29638955 | −0.0057081 | 0.00570806 |
| 3231 | 15 | 77925883 | 77926025 | −0.0056747 | 0.00567472 |
| 3232 | 15 | 70550847 | 70551302 | −0.0056732 | 0.00567323 |
| 3233 | 16 | 68344514 | 68344689 | 0.00563379 | 0.005633787 |
| 3234 | 19 | 8407968 | 8408395 | 0.00558497 | 0.005584967 |
| 3235 | 8 | 103250682 | 103250838 | −0.0055817 | 0.00558166 |
| 3236 | 21 | 34696604 | 34696778 | −0.0055575 | 0.00555749 |
| 3237 | 20 | 46634883 | 46635087 | −0.0055419 | 0.0055419 |
| 3238 | 19 | 2427327 | 2427474 | −0.0054856 | 0.00548561 |
| 3239 | 11 | 71639182 | 71639322 | −0.0054726 | 0.0054726 |
| 3240 | 19 | 3557779 | 3557937 | −0.0054331 | 0.00543311 |
| 3241 | 16 | 31053417 | 31053574 | 0.00543013 | 0.00543013 |
| 3242 | 3 | 13462675 | 13462815 | 0.00542732 | 0.005427323 |
| 3243 | 8 | 61193638 | 61193781 | 0.00535445 | 0.005354452 |
| 3244 | 19 | 46389464 | 46389658 | −0.0053412 | 0.00534115 |
| 3245 | 11 | 125495506 | 125495645 | −0.0053346 | 0.00533465 |
| 3246 | 2 | 21022507 | 21022644 | 0.00531497 | 0.005314966 |
| 3247 | 1 | 153643399 | 153643718 | −0.0052802 | 0.00528017 |
| 3248 | 12 | 120315051 | 120315203 | 0.00522433 | 0.005224329 |
| 3249 | 11 | 124746229 | 124746405 | −0.0052135 | 0.00521351 |
| 3250 | 2 | 85635717 | 85635991 | −0.0052081 | 0.00520811 |
| 3251 | 1 | 213123595 | 213123756 | −0.0052001 | 0.00520008 |
| 3252 | 1 | 12678130 | 12678274 | 0.00519273 | 0.005192726 |
| 3253 | 6 | 788168 | 788321 | 0.00515805 | 0.00515805 |
| 3254 | 16 | 3096404 | 3096545 | −0.0051185 | 0.00511852 |
| 3255 | 22 | 40859137 | 40859546 | 0.00510543 | 0.005105427 |
| 3256 | 16 | 79634608 | 79634819 | 0.00505295 | 0.005052953 |
| 3257 | 5 | 52856384 | 52856624 | −0.005017 | 0.00501696 |
| 3258 | 8 | 86019278 | 86019493 | 0.00500699 | 0.005006994 |
| 3259 | 5 | 137368238 | 137368383 | 0.00498498 | 0.004984982 |
| 3260 | 6 | 116421781 | 116422200 | 0.00497368 | 0.004973679 |
| 3261 | 16 | 20753187 | 20753356 | −0.0049462 | 0.00494621 |
| 3262 | 5 | 112629495 | 112629681 | −0.0049178 | 0.00491784 |
| 3263 | 22 | 30752443 | 30752702 | 0.0049163 | 0.004916304 |
| 3264 | 22 | 31090153 | 31090302 | −0.004885 | 0.00488499 |
| 3265 | 11 | 33757492 | 33757638 | 0.00488273 | 0.004882733 |
| 3266 | 4 | 109541451 | 109541724 | 0.00485415 | 0.004854148 |
| 3267 | 2 | 54198258 | 54198446 | 0.00484471 | 0.004844715 |
| 3268 | 1 | 31313670 | 31314114 | −0.004842 | 0.00484204 |
| 3269 | 8 | 117886944 | 117887103 | 0.00479395 | 0.004793953 |
| 3270 | 19 | 55593461 | 55593994 | −0.0047566 | 0.00475661 |
| 3271 | 14 | 23564269 | 23564406 | −0.0047447 | 0.00474469 |
| 3272 | 15 | 43622912 | 43623051 | −0.0047339 | 0.0047339 |
| 3273 | 11 | 128775467 | 128775574 | 0.00472938 | 0.004729384 |
| 3274 | 1 | 44496934 | 44497092 | −0.0047293 | 0.0047293 |
| 3275 | 2 | 10263314 | 10263448 | 0.0047146 | 0.004714599 |
| 3276 | 2 | 162016964 | 162017117 | 0.00468741 | 0.004687414 |
| 3277 | 11 | 65660800 | 65661012 | 0.00464616 | 0.004646158 |
| 3278 | 5 | 179223375 | 179223537 | 0.00462379 | 0.004623787 |
| 3279 | 3 | 15140710 | 15140879 | 0.00459803 | 0.004598029 |
| 3280 | 6 | 151713100 | 151713262 | −0.0045687 | 0.00456874 |
| 3281 | 20 | 1472141 | 1472285 | 0.00455365 | 0.004553654 |
| 3282 | 3 | 52740074 | 52740227 | 0.00453392 | 0.004533924 |
| 3283 | 18 | 47792840 | 47793011 | 0.00451226 | 0.004512258 |
| 3284 | 11 | 61583493 | 61583638 | −0.0044422 | 0.00444217 |
| 3285 | 15 | 69591491 | 69591638 | 0.00439706 | 0.004397059 |
| 3286 | 6 | 126307492 | 126307666 | −0.0043644 | 0.0043644 |
| 3287 | 11 | 67085104 | 67085410 | 0.0043609 | 0.004360897 |
| 3288 | 12 | 51984425 | 51984587 | 0.00435055 | 0.004350547 |
| 3289 | 2 | 219271983 | 219272170 | −0.0043021 | 0.00430206 |
| 3290 | 19 | 14583203 | 14583342 | 0.00428583 | 0.004285831 |
| 3291 | 19 | 12848199 | 12848415 | −0.0042704 | 0.00427037 |
| 3292 | 2 | 136743344 | 136743525 | 0.00424304 | 0.00424304 |
| 3293 | 1 | 228594894 | 228595031 | 0.0042259 | 0.004225904 |
| 3294 | 19 | 13227563 | 13227779 | −0.0042087 | 0.00420865 |
| 3295 | 1 | 111506432 | 111506785 | 0.00417175 | 0.004171747 |
| 3296 | 16 | 66982042 | 66982743 | −0.0041233 | 0.00412333 |
| 3297 | 1 | 118471859 | 118472009 | 0.00411733 | 0.004117333 |
| 3298 | 4 | 158141857 | 158142021 | 0.00410577 | 0.004105772 |
| 3299 | 17 | 28443565 | 28443718 | −0.0041007 | 0.00410067 |
| 3300 | 4 | 37684570 | 37684744 | −0.0040898 | 0.0040898 |
| 3301 | 7 | 100472675 | 100472841 | −0.0040471 | 0.00404708 |
| 3302 | 17 | 61523997 | 61524133 | 0.0040381 | 0.004038096 |
| 3303 | 6 | 36410676 | 36410847 | 0.00398575 | 0.003985747 |
| 3304 | 10 | 50970348 | 50970520 | 0.00397032 | 0.003970317 |
| 3305 | 3 | 125029701 | 125029845 | −0.0039556 | 0.00395555 |
| 3306 | 1 | 25291797 | 25291928 | 0.00395365 | 0.003953651 |
| 3307 | 6 | 26159047 | 26159215 | 0.00395262 | 0.003952616 |
| 3308 | 5 | 92917047 | 92917193 | 0.00394001 | 0.00394001 |
| 3309 | 4 | 331542 | 331697 | −0.0039283 | 0.00392831 |
| 3310 | 17 | 38519060 | 38519210 | 0.00389199 | 0.003891991 |
| 3311 | 15 | 64752711 | 64752829 | −0.0038728 | 0.00387278 |
| 3312 | X | 40125932 | 40126229 | 0.00386512 | 0.003865123 |
| 3313 | 2 | 179343179 | 179343330 | −0.0038521 | 0.00385213 |
| 3314 | 20 | 1878731 | 1878872 | 0.00384051 | 0.003840513 |
| 3315 | 14 | 20811487 | 20811713 | −0.0038046 | 0.00380456 |
| 3316 | 3 | 31268945 | 31269077 | −0.0037772 | 0.00377723 |
| 3317 | 17 | 76380197 | 76380463 | 0.00376392 | 0.003763915 |
| 3318 | 15 | 76196083 | 76196220 | −0.0037461 | 0.00374607 |
| 3319 | 19 | 12175425 | 12175572 | −0.0037116 | 0.00371155 |
| 3320 | 17 | 7138008 | 7138147 | −0.003665 | 0.00366496 |
| 3321 | 12 | 121838076 | 121838221 | 0.00366459 | 0.003664593 |
| 3322 | 12 | 50505442 | 50505589 | −0.0036626 | 0.00366255 |
| 3323 | 7 | 102004219 | 102004375 | −0.0036602 | 0.00366023 |
| 3324 | 3 | 4892148 | 4892293 | −0.00366 | 0.00365997 |
| 3325 | 17 | 75663124 | 75663403 | −0.0036581 | 0.00365811 |
| 3326 | 2 | 197790872 | 197791013 | 0.00364995 | 0.003649948 |
| 3327 | 19 | 2785593 | 2785754 | −0.0035348 | 0.00353479 |
| 3328 | 22 | 27053127 | 27053295 | −0.0035136 | 0.00351356 |
| 3329 | 14 | 50998842 | 50998985 | 0.00350956 | 0.003509564 |
| 3330 | 7 | 41982716 | 41982855 | −0.0035031 | 0.0035031 |
| 3331 | 19 | 37019094 | 37019374 | −0.0034772 | 0.00347721 |
| 3332 | 11 | 75921732 | 75921888 | 0.00342307 | 0.003423071 |
| 3333 | 4 | 3294320 | 3294468 | −0.0034139 | 0.00341392 |
| 3334 | 14 | 62583882 | 62584091 | −0.0033714 | 0.00337144 |
| 3335 | 4 | 140477425 | 140477573 | −0.0033644 | 0.00336436 |
| 3336 | 10 | 30990361 | 30990643 | −0.0033542 | 0.00335421 |
| 3337 | 7 | 29310671 | 29310813 | −0.0032191 | 0.00321906 |
| 3338 | 2 | 28113226 | 28113518 | 0.00319065 | 0.003190647 |
| 3339 | 7 | 766110 | 766464 | −0.00319 | 0.00319001 |
| 3340 | 1 | 94549302 | 94549470 | −0.0031847 | 0.00318467 |
| 3341 | 13 | 110958898 | 110959048 | −0.0031233 | 0.00312334 |
| 3342 | X | 597928 | 598097 | −0.0030769 | 0.00307689 |
| 3343 | 2 | 114457811 | 114457979 | 0.00305707 | 0.003057073 |
| 3344 | 10 | 106399442 | 106399585 | −0.0030435 | 0.00304345 |
| 3345 | 9 | 133588018 | 133588161 | −0.0030068 | 0.00300678 |
| 3346 | 14 | 59105304 | 59105464 | 0.00297012 | 0.002970124 |
| 3347 | 5 | 33936386 | 33936532 | −0.0029273 | 0.00292734 |
| 3348 | 3 | 66024477 | 66024627 | −0.0029143 | 0.00291427 |
| 3349 | 18 | 6633469 | 6633606 | 0.00285992 | 0.002859922 |
| 3350 | 3 | 154145791 | 154145930 | 0.00280712 | 0.002807116 |
| 3351 | 7 | 100813219 | 100813380 | 0.00279832 | 0.002798319 |
| 3352 | 3 | 180397223 | 180397399 | −0.0027621 | 0.00276206 |
| 3353 | 19 | 53445720 | 53445865 | 0.00271149 | 0.002711489 |
| 3354 | 14 | 23298827 | 23299091 | −0.0027114 | 0.00271137 |
| 3355 | 15 | 91475625 | 91475961 | 0.00270006 | 0.002700065 |
| 3356 | 7 | 27252689 | 27253042 | −0.0026992 | 0.00269925 |
| 3357 | 3 | 183736005 | 183736158 | 0.00269571 | 0.002695709 |
| 3358 | 8 | 22435965 | 22436238 | 0.0026882 | 0.002688202 |
| 3359 | 1 | 182583990 | 182584125 | 0.00266975 | 0.002669755 |
| 3360 | 22 | 32026795 | 32027020 | 0.00266465 | 0.002664646 |
| 3361 | 8 | 123793254 | 123793392 | 0.00260173 | 0.002601726 |
| 3362 | 10 | 90031287 | 90031454 | 0.00259806 | 0.002598063 |
| 3363 | 3 | 48481558 | 48481842 | −0.0025932 | 0.0025932 |
| 3364 | 2 | 95825566 | 95825774 | −0.0025687 | 0.00256873 |
| 3365 | 17 | 48074032 | 48074175 | −0.0025601 | 0.0025601 |
| 3366 | 19 | 6737615 | 6737829 | 0.00255512 | 0.002555121 |
| 3367 | 19 | 44488044 | 44488186 | −0.0024884 | 0.00248837 |
| 3368 | 9 | 134273561 | 134273948 | −0.0024832 | 0.00248322 |
| 3369 | 17 | 61909048 | 61909206 | −0.0024788 | 0.00247877 |
| 3370 | 10 | 50817732 | 50817898 | −0.0024703 | 0.00247033 |
| 3371 | 1 | 32237512 | 32238693 | −0.0024548 | 0.00245477 |
| 3372 | 12 | 66460076 | 66460219 | −0.0023959 | 0.00239588 |
| 3373 | 17 | 80477057 | 80477204 | 0.00237591 | 0.002375907 |
| 3374 | 12 | 94853421 | 94853765 | 0.00234918 | 0.00234918 |
| 3375 | 15 | 64386169 | 64386316 | 0.00229006 | 0.002290064 |
| 3376 | 1 | 20960125 | 20960275 | 0.00228148 | 0.002281484 |
| 3377 | 16 | 14091839 | 14091995 | −0.0022617 | 0.00226165 |
| 3378 | 2 | 39444073 | 39444253 | −0.0022606 | 0.00226062 |
| 3379 | 15 | 89951760 | 89953021 | −0.0022429 | 0.00224289 |
| 3380 | 1 | 156470822 | 156471084 | 0.00224126 | 0.002241256 |
| 3381 | 12 | 3601228 | 3601368 | 0.00222559 | 0.002225595 |
| 3382 | 1 | 10753755 | 10753913 | 0.00222216 | 0.002222157 |
| 3383 | 6 | 29596135 | 29596251 | 0.00221456 | 0.002214556 |
| 3384 | 3 | 37847781 | 37847994 | −0.0021838 | 0.00218379 |
| 3385 | 1 | 249132699 | 249132835 | −0.0021801 | 0.00218008 |
| 3386 | 17 | 55676747 | 55676892 | 0.00214769 | 0.002147692 |
| 3387 | 2 | 172290498 | 172290644 | −0.0021433 | 0.00214335 |
| 3388 | 19 | 54704552 | 54705040 | 0.00210774 | 0.00210774 |
| 3389 | 1 | 47800090 | 47800232 | 0.00209696 | 0.002096963 |
| 3390 | 11 | 117667050 | 117668144 | −0.0020969 | 0.00209689 |
| 3391 | 14 | 69620354 | 69620528 | 0.00208329 | 0.002083291 |
| 3392 | 15 | 99193850 | 99194102 | 0.00200741 | 0.002007411 |
| 3393 | 15 | 55488732 | 55488883 | −0.0019695 | 0.0019695 |
| 3394 | 6 | 7540994 | 7541132 | 0.00194547 | 0.001945466 |
| 3395 | 11 | 73019402 | 73019753 | −0.001937 | 0.001937 |
| 3396 | 2 | 46770239 | 46770414 | 0.00190381 | 0.001903808 |
| 3397 | 7 | 76178467 | 76178708 | −0.001854 | 0.00185403 |
| 3398 | 2 | 176979545 | 176979828 | −0.0018499 | 0.00184992 |
| 3399 | 1 | 23946192 | 23946349 | 0.00181374 | 0.001813741 |
| 3400 | 5 | 108063964 | 108064104 | −0.0017972 | 0.00179719 |
| 3401 | 19 | 58125482 | 58125656 | −0.0017427 | 0.00174275 |
| 3402 | 2 | 55459548 | 55459862 | 0.00172761 | 0.001727614 |
| 3403 | 9 | 127020042 | 127020160 | −0.0017236 | 0.00172356 |
| 3404 | 7 | 103086725 | 103086911 | 0.00168309 | 0.001683087 |
| 3405 | 3 | 42003644 | 42003789 | 0.00165691 | 0.00165691 |
| 3406 | 7 | 130034410 | 130034546 | 0.00162819 | 0.001628185 |
| 3407 | 13 | 46755257 | 46755410 | 0.00160338 | 0.001603383 |
| 3408 | 3 | 130612603 | 130612887 | 0.00158485 | 0.001584849 |
| 3409 | 6 | 137808750 | 137810520 | −0.0015725 | 0.00157247 |
| 3410 | 9 | 111696332 | 111696465 | 0.00156795 | 0.001567954 |
| 3411 | 16 | 2042025 | 2042177 | 0.00154739 | 0.001547391 |
| 3412 | 14 | 24701316 | 24701454 | 0.00154083 | 0.001540831 |
| 3413 | 2 | 11270112 | 11270272 | −0.0015396 | 0.00153963 |
| 3414 | 10 | 14995966 | 14996111 | −0.0015319 | 0.00153193 |
| 3415 | 19 | 46498311 | 46498395 | −0.0014906 | 0.0014906 |
| 3416 | 7 | 100183465 | 100183679 | −0.0014506 | 0.00145056 |
| 3417 | 3 | 12883296 | 12883538 | −0.0014299 | 0.00142994 |
| 3418 | 10 | 106113095 | 106113251 | 0.00140995 | 0.001409947 |
| 3419 | 17 | 17206937 | 17207086 | 0.00140115 | 0.001401146 |
| 3420 | 7 | 4848688 | 4849273 | −0.0013971 | 0.00139712 |
| 3421 | 10 | 126606480 | 126606608 | −0.0013501 | 0.00135012 |
| 3422 | 1 | 180203466 | 180203653 | −0.0013392 | 0.00133916 |
| 3423 | 12 | 106642580 | 106642735 | −0.0013197 | 0.00131971 |
| 3424 | 11 | 47290807 | 47290968 | −0.0013148 | 0.00131478 |
| 3425 | 17 | 1419284 | 1419428 | −0.0012976 | 0.00129761 |
| 3426 | 2 | 71192075 | 71192529 | −0.001222 | 0.00122197 |
| 3427 | 10 | 100206744 | 100206893 | 0.00118169 | 0.001181687 |
| 3428 | 6 | 4021278 | 4021464 | −0.0011747 | 0.00117469 |
| 3429 | 20 | 34680604 | 34680735 | −0.0011704 | 0.00117042 |
| 3430 | 5 | 174905539 | 174905675 | −0.0011612 | 0.00116116 |
| 3431 | 3 | 156272831 | 156273156 | −0.0011498 | 0.00114985 |
| 3432 | 1 | 59407642 | 59407786 | −0.0011185 | 0.00111851 |
| 3433 | 3 | 140985907 | 140986230 | 0.00110662 | 0.001106618 |
| 3434 | 5 | 128110288 | 128110533 | −0.0009822 | 0.000982246 |
| 3435 | 10 | 112257867 | 112258017 | −0.0008832 | 0.0008832 |
| 3436 | 19 | 1489577 | 1489744 | −0.0008814 | 0.000881385 |
| 3437 | 22 | 45060064 | 45060406 | −0.0007911 | 0.000791072 |
| 3438 | 19 | 57831561 | 57831919 | 0.00070428 | 0.000704283 |
| 3439 | 5 | 151138297 | 151138486 | 0.00069822 | 0.000698222 |
| 3440 | 17 | 27333028 | 27333267 | −0.0006879 | 0.000687893 |
| 3441 | 3 | 184337322 | 184337467 | −0.0006866 | 0.000686622 |
| 3442 | 16 | 4675576 | 4675730 | −0.0006787 | 0.000678726 |
| 3443 | 13 | 46038764 | 46039071 | −0.000666 | 0.000666042 |
| 3444 | 12 | 124086576 | 124086795 | 0.00063414 | 0.000634143 |
| 3445 | 1 | 97187736 | 97187883 | 0.00062874 | 0.000628736 |
| 3446 | 15 | 68126215 | 68126360 | 0.00062162 | 0.000621617 |
| 3447 | 7 | 24797284 | 24797430 | −0.0005885 | 0.000588515 |
| 3448 | 14 | 90147461 | 90147619 | −0.0005518 | 0.000551812 |
| 3449 | 3 | 37535763 | 37535918 | −0.0005482 | 0.000548194 |
| 3450 | 5 | 180634450 | 180634574 | 0.0005327 | 0.000532701 |
| 3451 | 19 | 58978157 | 58978292 | −0.0004957 | 0.000495723 |
| 3452 | 2 | 232573802 | 232573949 | −0.0004917 | 0.000491702 |
| 3453 | 3 | 124606425 | 124606578 | 0.00048228 | 0.000482282 |
| 3454 | 11 | 1569713 | 1569852 | 0.0004659 | 0.000465904 |
| 3455 | 17 | 79620588 | 79620835 | 0.00044871 | 0.000448711 |
| 3456 | 12 | 56694089 | 56694363 | −0.0004382 | 0.000438202 |
| 3457 | 6 | 100894965 | 100895279 | −0.0004295 | 0.000429507 |
| 3458 | 22 | 48963287 | 48963539 | 0.00042169 | 0.00042169 |
| 3459 | 11 | 120423203 | 120423410 | 0.00038709 | 0.000387088 |
| 3460 | 19 | 1237797 | 1237960 | 0.00031855 | 0.000318546 |
| 3461 | 8 | 118056968 | 118057115 | −0.0003111 | 0.000311103 |
| 3462 | 5 | 139944311 | 139944558 | −0.0002178 | 0.000217826 |
| 3463 | 5 | 32313230 | 32313502 | −0.0002046 | 0.000204559 |
| 3464 | 12 | 112546620 | 112546783 | 0.00020288 | 0.000202876 |
| 3465 | 7 | 135194611 | 135194749 | 0.00019245 | 0.000192448 |
| 3466 | 20 | 33146465 | 33146608 | 0.0001782 | 0.000178199 |
| 3467 | 14 | 100241364 | 100241518 | 0.00013597 | 0.000135974 |
| 3468 | 14 | 60097169 | 60097316 | −0.0001299 | 0.000129908 |
| 3469 | 4 | 186990247 | 186990402 | 0.00010727 | 0.000107274 |
| 3470 | X | 599993 | 600167 | −8.83E−05 | 8.83E−05 |
| 3471 | 17 | 28256788 | 28256937 | 7.80E−05 | 7.80E−05 |
| 3472 | 10 | 126107391 | 126107805 | 3.20E−05 | 3.20E−05 |

### Results

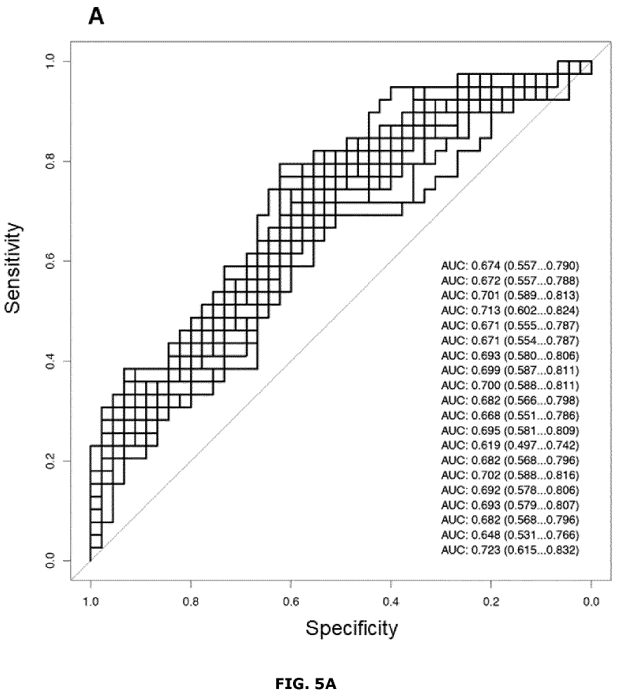

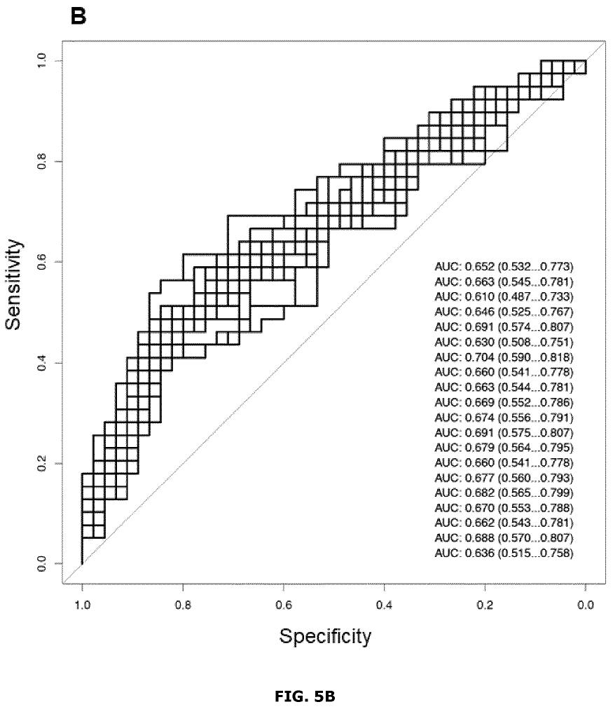

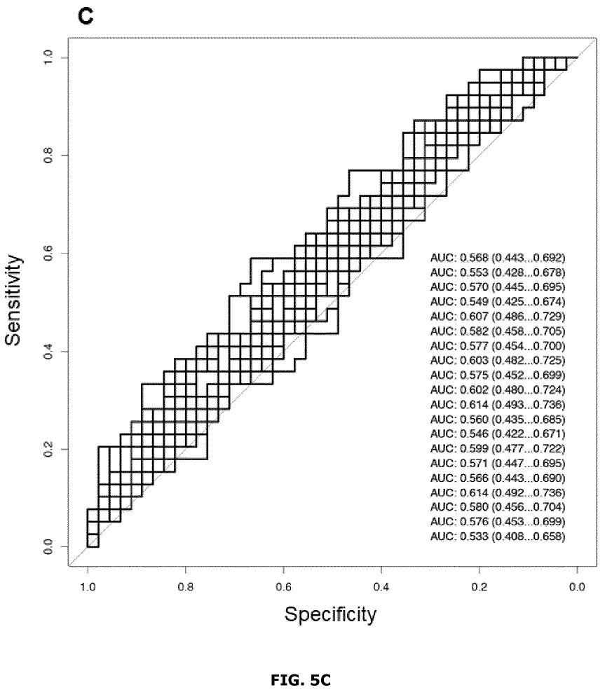

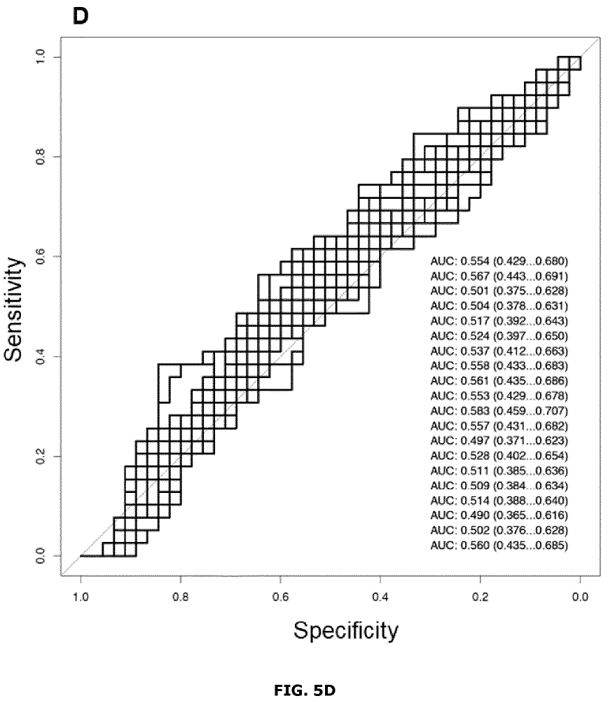

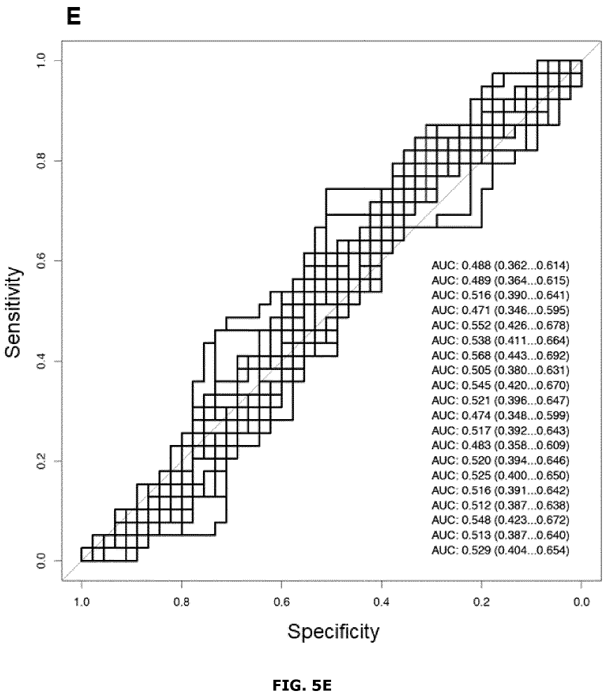

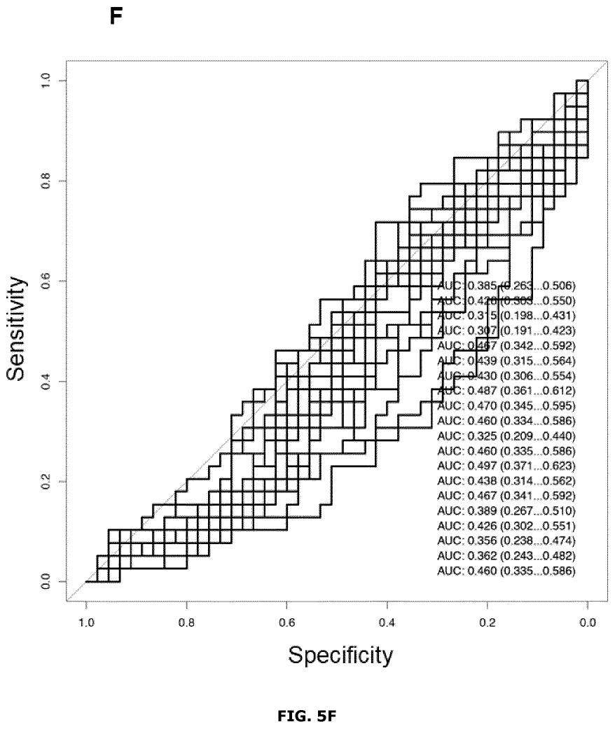

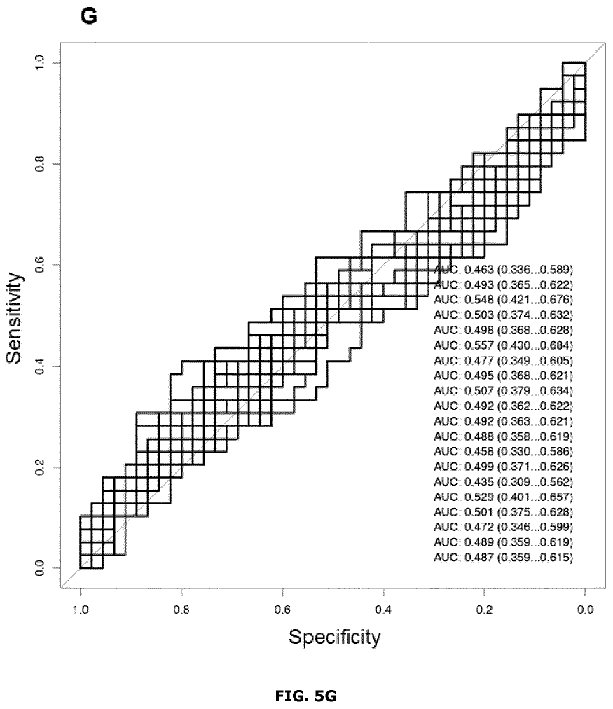

2872 genomic regions (N=SID ID NO 601 to SEQ ID 3472) were selected as having non-zero coefficients in the final ridge regression models. This model yielded an average AUC of 0.752. From these 2872 regions, a subset of regions was further randomly selected to assess model performance using a fraction of the selected genomic regions. The predictive value of models built using 1436 (FIG. 5A—mean AUC 0.684), 1149 (FIG. 5B—mean AUC 0.665), 862 (FIG. 5C—mean AUC 0.577), 288 (FIG. 5D—mean AUC 0.531), 29 (FIG. 5E—mean AUC 0.516), 15 (FIG. 5F—mean AUC 0.418) and 12 (FIG. 5G—mean AUC 0.494) randomly selected regions was determined. Models containing 288, 862, 1149 and 1436 were predictive of preeclampsia, as their average AUCs are significantly higher than a random AUC of 0.5 (One Sample t-test P<0.0001 for all comparisons).

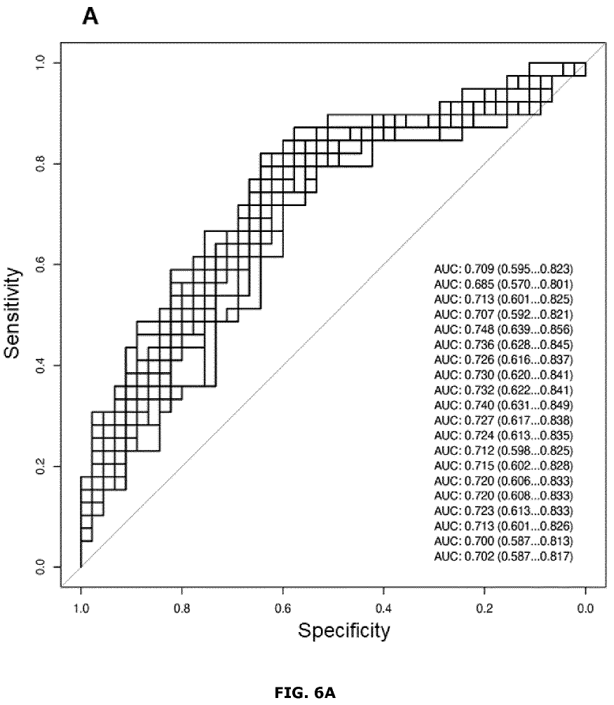

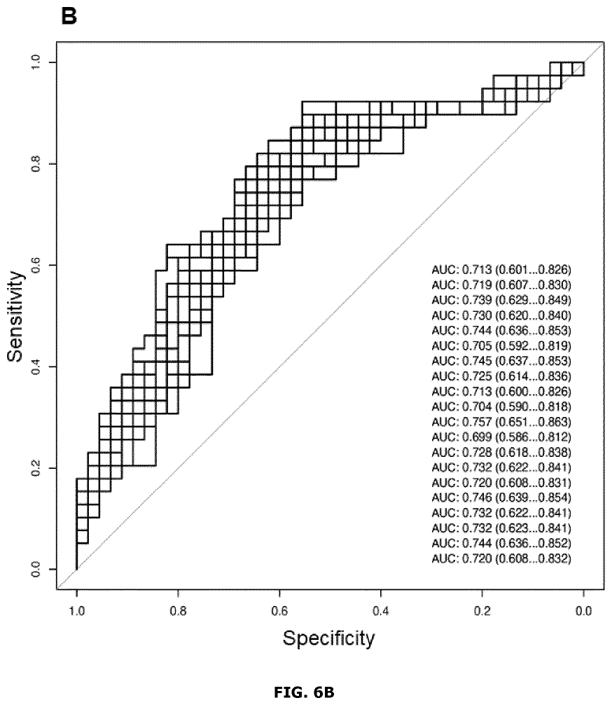

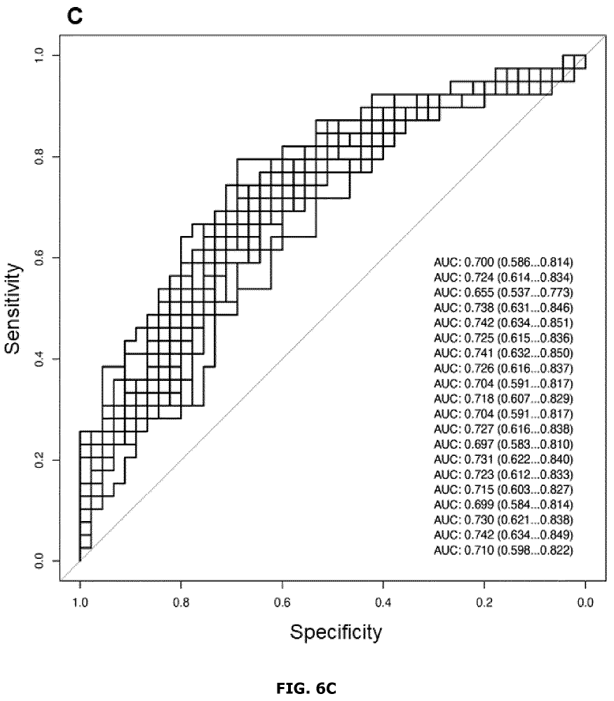

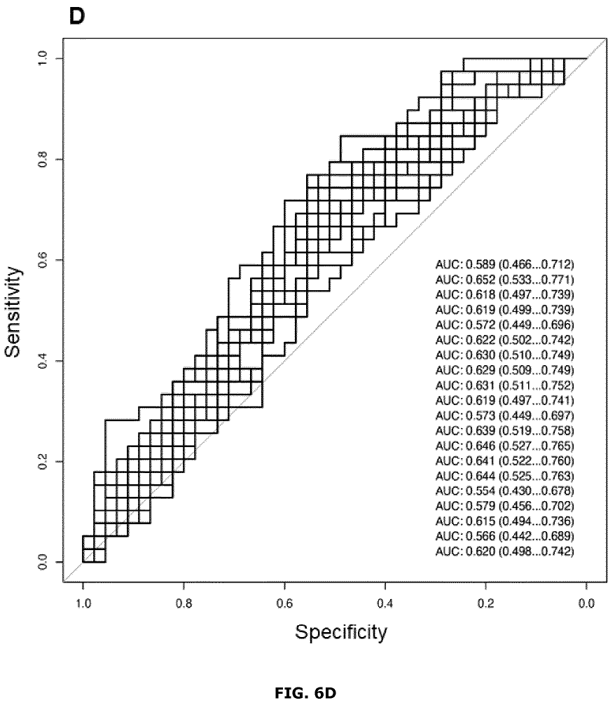

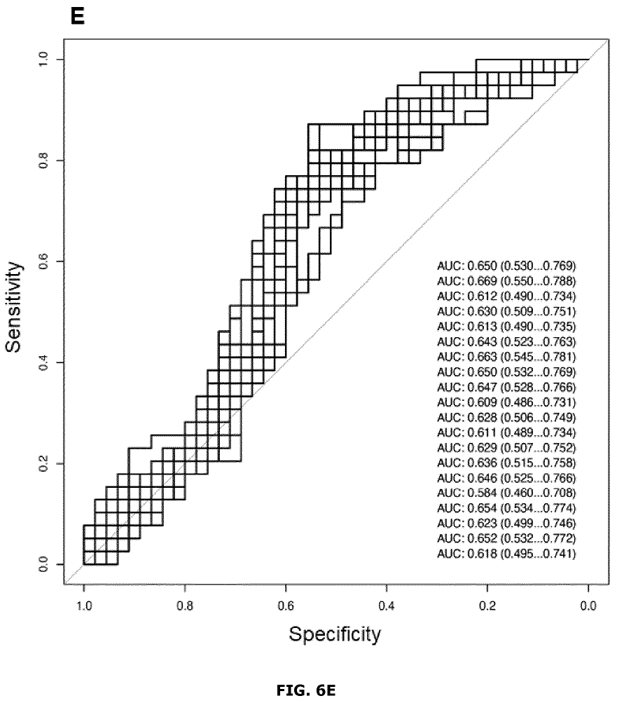

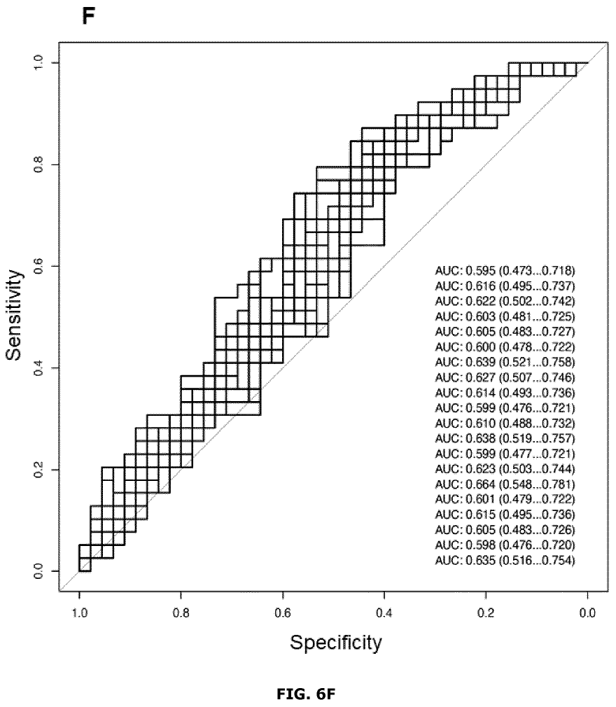

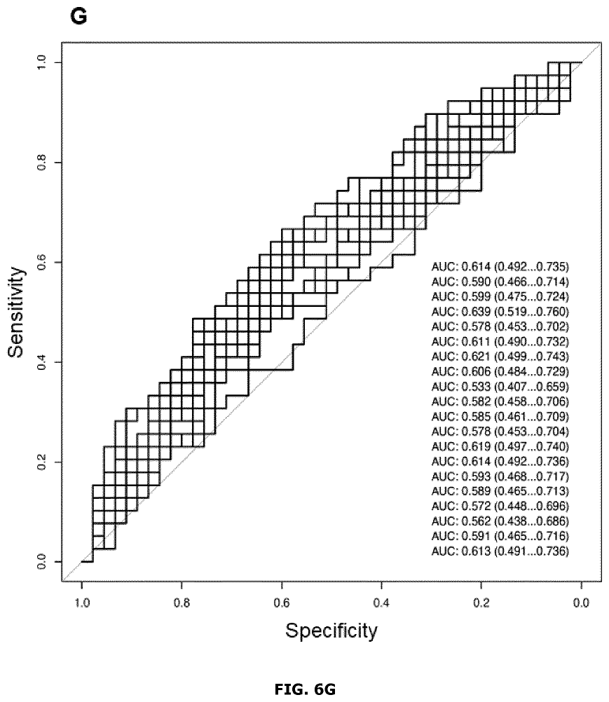

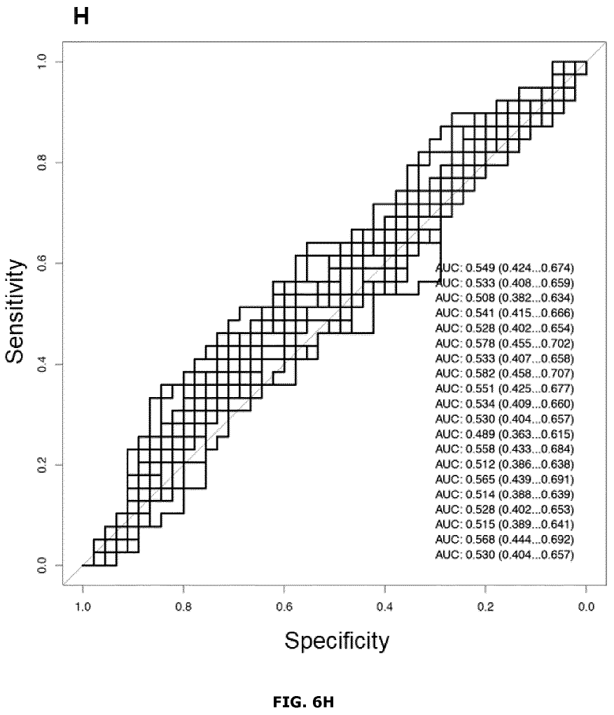

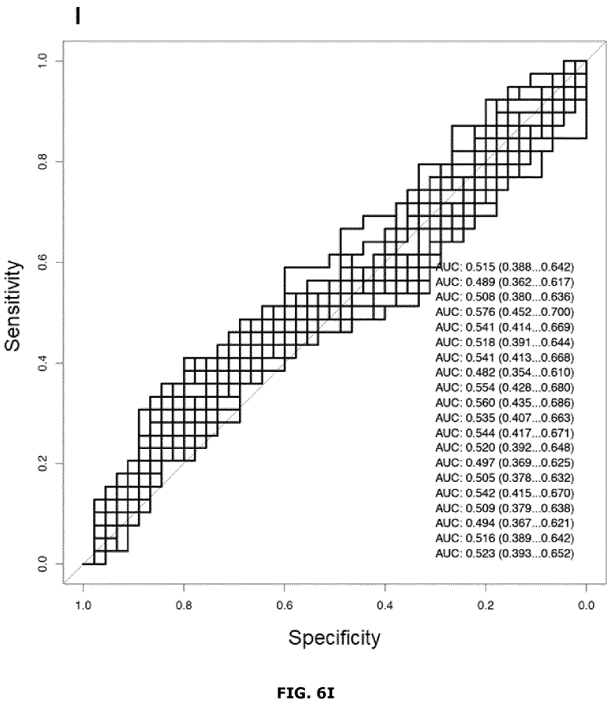

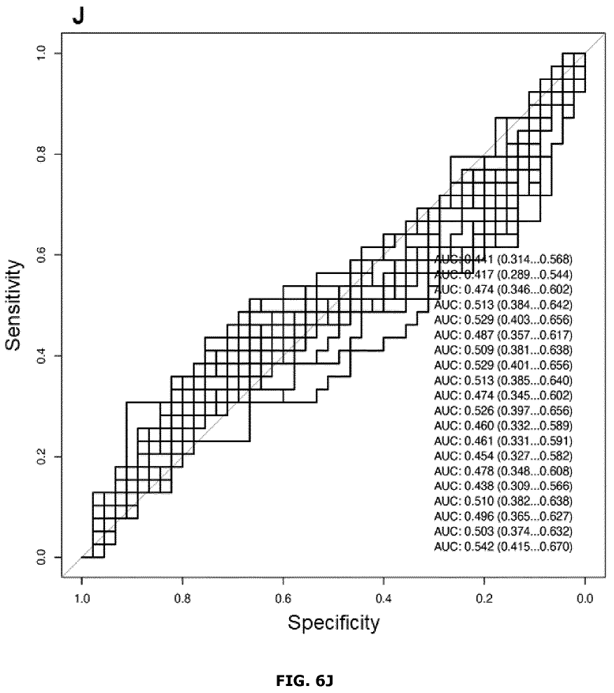

Predictive value of models built using (i) the 1436 regions with the highest absolute coefficient (FIG. 6A—SEQ ID NO 601 to 2036—mean AUC 0.719); (ii) the 1149 regions with the highest absolute coefficient (FIG. 6B—SEQ ID NO 601 to 1749—mean AUC 0.727); (iii) the 862 regions with the highest absolute coefficient (FIG. 6C—SEQ ID NO 601 to 1462—mean AUC 0.718); (iv) the 288 regions with the highest absolute coefficient (FIG. 6D—SEQ ID NO 601 to 888 mean AUC 0.613); (v) the 29 regions with the highest absolute coefficient (FIG. 6E—SEQ ID NO 601 to 629—mean AUC 0.633); (vi) the 15 regions with the highest absolute coefficient (FIG. 6F—SEQ ID NO 601 to 615—mean AUC 0.615); (vii) the 12 regions with the highest absolute coefficient (FIG. 6G—SEQ ID NO 601 to 612 mean AUC 0.5944); (viii) the 11 regions with the highest absolute coefficient (FIG. 6H—SEQ ID NO 601 to 611—mean AUC 0.594); (ix) the 10 regions with the highest absolute coefficient (FIG. 6I—SEQ ID NO 601 to 610—mean AUC 0.523); the 9 regions with the highest absolute coefficient (FIG. 6J—SEQ ID NO 601 to 609—mean AUC 0.481) was determined. Models containing more than the top 10 highest-ranking regions were hence predictive of preeclampsia, as their average AUCs are significantly higher than a random AUC of 0.5 (One Sample t-test P<0.00001 for all comparisons).

In conclusion, although cfDNA methylation as measured at 2872 genomic regions produced the highest predictive value, with an AUC of 0.752, also smaller subsets of these regions were sufficient to predict preeclampsia, with for example more than 10 top-ranking regions or 288 randomly selected regions out of the 2872 regions identified yielding a significant predictive power to predict preeclampsia around 12 weeks of gestation.

1. A method for the prediction of the risk of developing preeclampsia in a pregnant human subject, wherein said subject's gestational age is under 140 days, comprising the steps of:
a. measuring in a sample from said subject a plurality of loci-specific DNA methylation levels;
b. deriving from said measurement a DNA methylation profile for said subject;
c. comparing said DNA methylation profile to,
i. a reference DNA methylation profile corresponding to a high risk of preeclampsia group; and/or,
ii. a reference DNA methylation profile corresponding to a low risk of preeclampsia group; and,
d. assigning on the basis of said comparison said subject to a preeclampsia risk group, thereby predicting the risk of developing preeclampsia in said subject,
wherein said DNA is cell-free DNA.

2. The method according to claim 1, wherein said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 3472, as found in the human genome build Grch37/hg19.

3. The method according to claim 1 or 2, wherein said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 1768, as found in the human genome build Grch37/hg19, preferably defined in reference to one of SEQ ID NO: 601 to 1026, as found in the human genome build Grch37/hg19, more preferably defined in reference to one of SEQ ID NO: 601 to 652, as found in the human genome build Grch37/hg19.

4. The method according to any one of claims 1 to 3, wherein said measuring step comprises measuring a plurality of loci-specific DNA methylation levels in a set of genomic regions comprising 10 genomic regions wherein each of said 10 genomic regions is distinct and defined in reference to one of SEQ ID NO: 601 to 610, as found in the human genome build Grch37/hg19.

5. The method according to any one of claims 1 to 4, wherein said measuring step comprises measuring a plurality of loci-specific DNA methylation levels in a set of genomic regions comprising 11 genomic regions wherein each of said 11 genomic regions is distinct and defined in reference to one of SEQ ID NO: 601 to 611, as found in the human genome build Grch37/hg19, preferably wherein said measuring step comprises measuring a plurality of loci-specific DNA methylation levels in a set of genomic regions comprising 12 genomic regions wherein each of said 12 genomic regions is distinct and defined in reference to one of SEQ ID NO: 601 to 612, as found in the human genome build Grch37/hg19, more preferably wherein said measuring step comprises measuring a plurality of loci-specific DNA methylation levels in a set of genomic regions comprising 15 genomic regions wherein each of said 15 genomic regions is distinct and defined in reference to one of SEQ ID NO: 601 to 615, as found in the human genome build Grch37/hg19.

6. The method according to any one of claims 1 to 5, wherein said measuring step comprises measuring a plurality of loci-specific DNA methylation levels in a set of genomic regions comprising 29 genomic regions wherein each of said 29 genomic regions is distinct and defined in reference to one of SEQ ID NO: 601 to 629, as found in the human genome build Grch37/hg19.

7. The method according to any one of claims 1 to 6, wherein said sample is a blood sample.

8. The method according to any one of claims 1 to 7, wherein said DNA is circulating-cell-free DNA.

9. The method according to any one of claims 1 to 8, wherein said reference DNA methylation profile corresponding to a high risk of preeclampsia group is derived from the measure of said plurality of loci-specific DNA methylation levels in control subjects, preferably gestational age-matched control subjects, that developed preeclampsia at later stage of pregnancy.

10. The method according to any one of claims 1 to 9, wherein said reference DNA methylation profile corresponding to a low risk of preeclampsia group is derived from the measure of said plurality of loci-specific DNA methylation levels in control subjects, preferably gestational age-matched control subjects, that remained healthy in respect to preeclampsia during their pregnancy.

11. The method according to any one of claims 1 to 10, wherein said subject's gestational age is under 133 days, preferably under 126 days or under 119 days, more preferably under 112 days.

12. The method according to any one of claims 1 to 11, wherein said subject's gestational age is ranging from 28 days to 111 days, preferably is ranging from 63 days to 104 days.

13. The method according to any one of claims 1 to 12, wherein said DNA methylation is methylation of cytosine, preferably methylation of cytosine in a CpG dinucleotide.

14. A kit comprising a set of capture probes specific for at least 10 genomic regions, wherein each of said at least 10 genomic regions are distinct and defined in reference to one of SEQ ID NO: 601 to 3472, as found in the human genome build Grch37/hg19, preferably in reference to one of SEQ ID NO: 601 to 610, as found in the human genome build Grch37/hg19.

15. Use of the kit according to claim 14 for the prediction of the risk of developing preeclampsia in a human subject.
